(19) United States
(12) Patent Application Publication
(10) Pub. No.: US 2021/0371388 A1
(43) Pub. Date: Dec. 2, 2021

(54) Bicyclic Carboxamide with Exocyclic Urea Derivatives as Antivirals for the Treatment of HBV Infection

(71) Applicant: Baruch S. Blumberg Institute, Doylestown, PA (US)

(72) Inventors: Yanming Du, Cheshire, CT (US); Ju-Tao Guo, Lansdale, PA (US); Nicky Hwang, Doylestown, PA (US)

(21) Appl. No.: 17/318,680

(22) Filed: May 12, 2021

Related U.S. Application Data
(60) Provisional application No. 63/023,339, filed on May 12, 2020.

(51) Int. Cl. C07D 241/52; C07D 241/48; C07D 215/233; C07D 279/16; C07D 265/36; C07D 241/04

(52) U.S. Cl. CPC C07D 241/52; C07D 241/48; C07D 241/04; C07D 279/16; C07D 265/36; C07D 215/233

(57) ABSTRACT

Pharmaceutical compositions of the invention comprise functionalized bicyclic carboxamide useful as pregenomic RNA encapsidation inhibitors, useful for the treatment of Hepatitis B virus (HBV) infection.

### CROSS-REFERENCE TO RELATED APPLICATION

The present application claims the benefit of priority from U.S. Provisional Application No. 63/023,339, filed May 12, 2020, the contents of which are hereby incorporated by reference herein in their entirety.

### STATEMENT OF GOVERNMENT INTEREST

This invention was made with government support under R01AI113267 awarded by the National Institute of Health. The government has certain rights in the invention.

### FIELD OF THE DISCLOSURE

The present invention describes novel compounds and novel methods of use of compounds as pregenomic RNA encapsidation inhibitors, useful for the treatment of Hepatitis B virus (HBV) infection and related conditions.

### BACKGROUND

Hepatitis B virus (HBV) is a member of the Hepadnaviridae family and contains a 3.2 kb, partially double-stranded, relaxed circular (rc) DNA genome. Hepatitis B virus (HBV) has infected one-third of world population, and 240 million people are chronic carriers, to whom a curative therapy is still not available. Approximately one-third of these individuals will die from serious liver diseases, such as cirrhosis and hepatocellular carcinoma, if left untreated (Lee, 1997; Lok, 2004).

Seven drugs are currently available for the management of chronic hepatitis B, which include two formulations of alpha-interferon (standard and pegylated) and five nucleos(t)ide analogues (lamivudine, adefovir, entecavir, telbivudine, and tenofovir) that inhibit HBV DNA polymerase (Keeffe et al., 2008). At present, the preferred first-line treatment choices are entecavir, tenofovir or peg-interferon alfa-2a. However, even with the first-line treatment options, peg-interferon alfa-2a is effective in achieving certain serological milestones in only one-third of treated patients and frequently associated with severe side effects (Janssen et al., 2005; Lau et al., 2005; Perrillo, 2009). Entecavir and tenofovir are highly potent HBV inhibitors, but a long-term or possibly life-time treatment is required to continuously suppress HBV replication, which may eventually fail due to emergence of drug resistant viruses (Dienstag, 2009). Hence, there is a pressing need for the introduction of novel, safe and effective therapies for chronic hepatitis B, which is listed by National Institute of Allergy and Infectious Diseases (NIAID) as a High Priority Area of Interest.

Hepatitis B virus (HBV) core protein assembles viral pre-genomic (pg) RNA and DNA polymerase into nucleocapsids for reverse transcriptional DNA replication to take place. Pregenomic (pg) RNA is the template for reverse transcriptional replication of HBV DNA and its encapsidation, together with viral DNA polymerase, into nucleocapsid is essential for the subsequent viral DNA synthesis. Inhibition of pregenomic RNA (pg) encapsidation would block HBV replication and provide a new therapeutic approach to the treatment of HBV.

There is a long felt need for new antiviral drugs that are both disease-modifying and effective in treating patients that are infected with hepatitis B virus. There is also a clear and present need for new antiviral drugs that are both disease modifying and effective in treating patients that are infected with drug resistant hepatitis B virus. The present invention addresses the need for new antiviral drugs that are both disease-modifying and effective in treating patients that are infected with hepatitis B virus.

### BRIEF SUMMARY

The present invention is directed towards Bicyclic carboxamide with Exocyclic Urea Derivatives of the formula (I), useful as pregenomic RNA encapsidation inhibitors of HBV for the treatment of Hepatitis B virus (HBV) infection and related conditions.

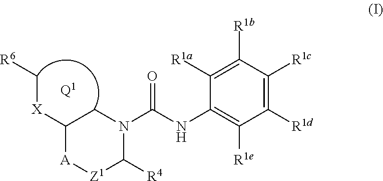

Including enantiomers, diasteromers, hydrates, solvates, pharmaceutically acceptable salts, isotopic analogs, prodrugs and complexes thereof, wherein:
A is selected from a group consisting of CH₂, carbonyl (C═O), oxygen,

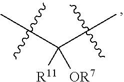

### and NR^{7a};

Z¹ is selected from the group consisting of a bond, carbonyl (C═O),

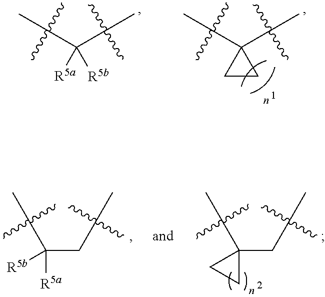

n¹ is 1, 2, 3, 4, or 5;
n² is 1, 2, 3, 4, or 5;
When A is carbonyl (C═O), Z¹ is not carbonyl (C═O);
The ring designated Q¹ is selected from the group consisting of an aromatic ring containing 5 ring atoms, an aromatic ring containing 6 ring atoms, a saturated ring containing 5 ring atoms, a saturated ring containing 6 ring atoms, and a saturated ring containing 7 ring atoms;
When the ring designated Q¹ is an aromatic ring containing 6 ring atoms, X is selected from the group consisting CR⁸ and nitrogen;
When the ring designated Q¹ is an aromatic ring containing 5 ring atoms, X is nitrogen;
When the ring designated Q¹ is a saturated ring containing 5 ring atoms, X is selected from a group consisting of CH₂, CHR⁸ and NR^{7b};
When the ring designated Q¹ is a saturated ring containing 6 ring atoms, X is selected from a group consisting of CH₂, CHR⁸ and NR^{7b};
When the ring designated Q¹ is a saturated ring containing 7 ring atoms, X is selected from a group consisting of CH₂, CHR⁸ and NR^{7b};
R^{1a} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1b} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1c} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1d} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1e} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1b} and R^{1c} are optionally taken together with the atom to which they are bound to form an optionally substituted ring having 5-7 ring atoms optionally containing up to 2 groups selected from oxygen and NH;
R^{1c} and R^{1d} are optionally taken together with the atom to which they are bound to form an optionally substituted ring having 5-7 ring atoms optionally containing up to 2 groups selected from oxygen and NH;
R⁴ is selected from the group consisting of hydrogen, C₁₋₆ linear alkyl, C₃₋₇ branched alkyl,

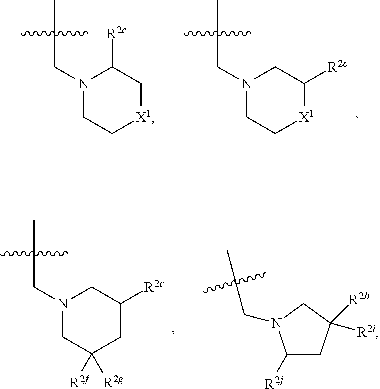

optionally substituted C₃₋₈ cycloalkyl, optionally substituted aryl, and optionally substituted heteroaryl;
X¹ is selected from the group consisting of oxygen, CR^{2a}R^{2b}, C═O, SO₂, and NSO₂R^{2d}.
R^{2a} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2b} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2c} is selected from the group consisting of hydrogen, CO₂R^{2e}, optionally substituted C₁₋₆ linear alkyl, and optionally substitute C₃₋₇ branched alkyl;
R^{2d} is selected from the group consisting of C₁₋₆ linear alkyl and C₃₋₇ branched alkyl;
R^{2e} is selected from the group consisting of hydrogen, C₁₋₆ linear alkyl, and C₃₋₇ branched alkyl;
R^{2f} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2g} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2h} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R²ⁱ is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2j} is selected from the group consisting of hydrogen, CO₂R^{2e}, optionally substituted C₁₋₆ linear alkyl, and optionally substitute C₃₋₇ branched alkyl;
R^{5a} is selected from the group consisting of hydrogen, C₁₋₆ linear alkyl, C₃₋₇ branched alkyl, CONH₂,

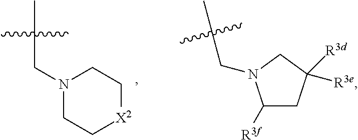

optionally substituted aryl, and optionally substituted heteroaryl;
R^{5b} is selected from a group consisting of hydrogen, C₁₋₆ linear alkyl, and C₃₋₇ branched alkyl;
X² is selected from the group consisting of oxygen, CR^{3a}R^{3b}, C═O, SO₂, NSO₂R^{3c},
R^{3a} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3b} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3c} is selected from the group consisting of C₁₋₆ linear alkyl and C₃₋₇ branched alkyl;
R^{3d} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3e} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3f} is selected from the group consisting of hydrogen, CO₂R^{3g}, optionally substituted C₁₋₆ linear alkyl, and optionally substitute C₃₋₇ branched alkyl;
R^{3g} is selected from the group consisting of hydrogen, C₁₋₆ linear alkyl, and C₃₋₇ branched alkyl;
R⁶ is selected from a group consisting of hydrogen, halogen, optionally substituted C₁₋₆ linear alkyl, optionally substituted C₃₋₇ branched alkyl, optionally substituted C₃₋₈ cycloalkyl, optionally substituted C₁₋₆ linear alkoxy, optionally substituted C₃₋₇ branched alkoxy, optionally substituted C₃₋₈ cycloalkoxy, —OBenzyl,

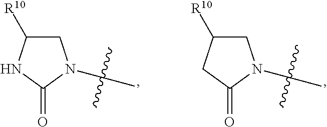

optionally substituted aryl, and optionally substituted heteroaryl;
R⁷ is selected from a group consisting of hydrogen, optionally substituted C₁₋₆ linear alkyl, optionally substituted C₃₋₇ branched alkyl, C₁₋₆ fluoroalkyl, C₃₋₇ branched fluoroalkyl optionally substituted C₁₋₆ alkenyl, CO₂R¹⁰, CONHR¹⁰, SO₂R¹⁰, and

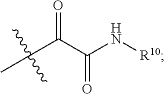

R^{7a} is selected from a group consisting of hydrogen, optionally substituted C₁₋₆ linear alkyl, optionally substituted C₃₋₇ branched alkyl, C₁₋₆ fluoroalkyl, C₃₋₇ branched fluoroalkyl, optionally substituted C₁₋₆ alkenyl, CO₂R¹⁰, CONHR¹⁰, SO₂R¹⁰, and

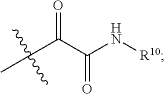

R^{7b} is selected from a group consisting of hydrogen, optionally substituted C₁₋₆ linear alkyl, optionally substituted C₃₋₇ branched alkyl, C₁₋₆ fluoroalkyl, C₃₋₇ branched fluoroalkyl, optionally substituted C₁₋₆ alkenyl, CO₂R¹⁰, CONHR¹⁰, SO₂R¹⁰, and

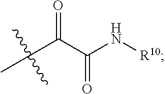

R⁸ is selected from a group consisting of hydrogen, halogen, optionally substituted C₁₋₆ linear alkyl, optionally substituted C₃₋₇ branched alkyl, optionally substituted C₃₋₈ cycloalkyl, optionally substituted aryl, optionally substituted heteroaryl, CO₂R¹⁰, CONHR¹⁰, NHCOR¹⁰, SO₂R¹⁰, and

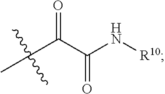

### When R⁸ is

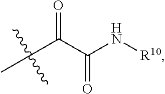

### R⁷ is not

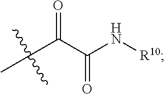

### When R⁸ is

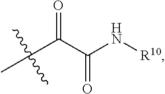

### R^{7a} is not

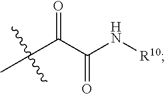

### When R⁸ is

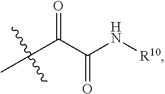

### R^{7b} is not

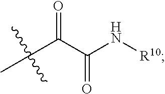

R⁹ is selected from a group consisting of hydrogen, optionally substituted C₁₋₄ alkyl, optionally substituted halo C₁₋₄ alkyl, and optionally substituted C₃₋₇ cycloalkyl;
R¹⁰ is selected from a group consisting of hydrogen, optionally substituted C₁₋₆ linear alkyl, optionally substituted C₃₋₇ branched alkyl, optionally substituted C₃₋₈ cycloalkyl, benzyl, optionally substituted aryl, and optionally substituted heteroaryl;
R¹¹ is selected from a group consisting of hydrogen, optionally substituted C₁₋₆ linear alkyl, optionally substituted C₃₋₇ branched alkyl, optionally substituted C₃₋₈ cycloalkyl, optionally substituted aryl, and optionally substituted heteroaryl.

The present invention further relates to compositions comprising: an effective amount of one or more compounds according to the present invention and an excipient.

The present invention also relates to a method for treating or preventing diseases that involve pregenomic RNA encapsidation, including, for example, HBV infection, said method comprising administering to a subject an effective amount of a compound or composition according to the present invention.

The present invention yet further relates to a method for treating or preventing diseases that involve pregenomic RNA encapsidation, including, for example, HBV infection, wherein said method comprises administering to a subject a composition comprising an effective amount of one or more compounds according to the present invention and an excipient.

The present invention also relates to a method for treating or preventing disease or conditions associated with HBV infection, and diseases that involve pregenomic RNA encapsidation. Said methods comprise administering to a subject an effective amount of a compound or composition according to the present invention.

The present invention yet further relates to a method for treating or preventing disease or conditions associated with HBV infection, and diseases that involve pregenomic RNA encapsidation, wherein said method comprises administering to a subject a composition comprising an effective amount of one or more compounds according to the present invention and an excipient.

These and other objects, features, and advantages will become apparent to those of ordinary skill in the art from a reading of the following detailed description and the appended claims. All percentages, ratios and proportions herein are by weight, unless otherwise specified. All temperatures are in degrees Celsius (° C.) unless otherwise specified. All documents cited are in relevant part, incorporated herein by reference; the citation of any document is not to be construed as an admission that it is prior art with respect to the present invention.

### DETAILED DESCRIPTION

The pregenomic RNA encapsidation inhibitors of the present invention are capable of treating and preventing diseases associated with pregenomic RNA encapsidation, for example HBV infection. Pregenomic (pg) RNA is the template for reverse transcriptional replication of HBV DNA and its encapsidation, together with viral DNA polymerase, into nucleocapsid is essential for the subsequent viral DNA synthesis. Without wishing to be limited by theory, it is believed that inhibition of pregenomic RNA encapsidation can ameliorate, abate, or otherwise cause to be controlled, diseases associated with pregenomic RNA encapsidation, for example HBV infection. Pregenomic RNA encapsidation inhibitors of the present invention address the clear and unmet need to identify novel and safe antiviral agents for the treatment of HBV infection that are chemically and mechanistically distinct from HBV antiviral drugs in current clinical use.

Clinically, the pregenomic RNA encapsidation inhibitors of the present invention complement the current medications by providing an additional option for a subpopulation of patients that do not tolerate or benefit from the current medications (Akbar et al., 2009; Liaw, 2009; Peters, 2009; Wiegand, van Bommel, and Berg). In addition, the pregenomic RNA encapsidation inhibitors of the present invention may be effective on HBV variants that are resistant to the currently available DNA polymerase inhibitors (Zoulim and Locarnini, 2009). Further, combination therapies of the pregenomic RNA encapsidation inhibitors of the present invention with DNA polymerase inhibitors may synergistically suppress HBV replication and prevent the emergence of drug resistance, offering a safer and more effective treatment for chronic hepatitis B (Billioud et al., 2011).

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as a an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” or “aralkyl” refers to the group -alkyl-aryl, where the alkyl and aryl groups are as defined herein. Aralkyl groups of the present invention are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and/or “heterocycle” and/or “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

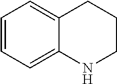

is, for the purposes of the present invention, considered a heterocyclic unit. 6,7-Dihydro-5H-cyclopentapyrimidine having the formula:

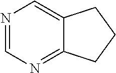

is, for the purposes of the present invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

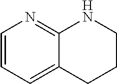

is, for the purposes of the present invention, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine(I)), —CN, —NO₂, oxo (═O), —OR¹², —SR¹², —N(R¹²)₂, —NR¹²C(O)R¹², —SO₂R¹², —SO₂OR¹², —SO₂N(R¹²)₂, —C(O)R¹², —C(O)OR¹², —C(O)N(R¹²)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R¹²; wherein R¹², at each occurrence, independently is hydrogen, —OR¹³, —SR¹³, —C(O)R¹³, —C(O)OR¹³, —C(O)N(R¹³)₂, —SO₂R¹³, —S(O)₂OR¹³, —N(R¹³)₂, —NR¹³C(O)R¹³, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R¹² units taken together with the atom(s) to which they are bound form an optionally substituted carbocycle or heterocycle wherein said carbocycle or heterocycle has 3 to 7 ring atoms; wherein R¹³, at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R¹³ units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR¹⁴; for example, —OH, —OCH₃, —OCH₂CH₃, —OCH₂CH₂CH₃;
ii) —C(O)R¹⁴; for example, —COCH₃, —COCH₂CH₃, —COCH₂CH₂CH₃;
iii) —C(O)OR¹⁴; for example, —CO₂CH₃, —CO₂CH₂CH₃, —CO₂CH₂CH₂CH₃;
iv) —C(O)N(R¹⁴)₂; for example, —CONH₂, —CONHCH₃, —CON(CH₃)₂;
v) —N(R¹⁴)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R¹⁴; for example, —SO₂H; —SO₂CH₃; —SO₂C₆H₅;
ix) C₁-C₆ linear, branched, or cyclic alkyl;
x) Cyano
xi) Nitro;
xii) N(R¹⁴)C(O)R¹⁴;
xiii) Oxo (═O);
xiv) Heterocycle; and
xv) Heteroaryl.
   wherein each R¹⁴ is independently hydrogen, optionally substituted C₁-C₆ linear or branched alkyl (e.g., optionally substituted C₁-C₄ linear or branched alkyl), or optionally substituted C₃-C₆ cycloalkyl (e.g optionally substituted C₃-C₄ cycloalkyl); or two R¹⁴ units can be taken together to form a ring comprising 3-7 ring atoms. In certain aspects, each R¹⁴ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of the present invention the terms “compound,” “analog,” and “composition of matter” stand equally well for the pregenomic RNA encapsidation inhibitors described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

The pregenomic RNA encapsidation inhibitors.

The pregenomic RNA encapsidation inhibitors of the present invention useful for the treatment of Hepatitis B virus (HBV) infection and related conditions are functionalized benzamide derivatives, and include all enantiomeric and diastereomeric forms and pharmaceutically accepted salts thereof having the formula (I):

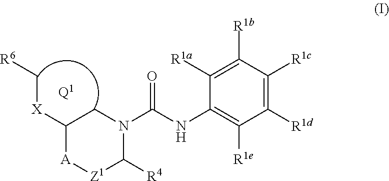

Including enantiomers, diasteromers, hydrates, solvates, pharmaceutically acceptable salts, isotopic analogs, prodrugs and complexes thereof, wherein:
A is selected from a group consisting of CH₂, carbonyl (C═O), oxygen,

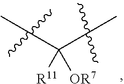

### and NR^{7a};

Z¹ is selected from the group consisting of a bond, carbonyl (C═O),

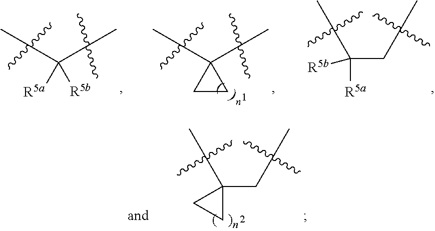

n¹ is 1, 2, 3, 4, or 5;
n² is 1, 2, 3, 4, or 5;
When A is carbonyl (C═O), Z¹ is not carbonyl (C═O);
The ring designated Q¹ is selected from the group consisting of an aromatic ring containing 5 ring atoms, an aromatic ring containing 6 ring atoms, a saturated ring containing 5 ring atoms, a saturated ring containing 6 ring atoms, and a saturated ring containing 7 ring atoms;
When the ring designated Q¹ is an aromatic ring containing 6 ring atoms, X is selected from the group consisting CR⁸ and nitrogen;
When the ring designated Q¹ is an aromatic ring containing 5 ring atoms, X is nitrogen;
When the ring designated Q¹ is a saturated ring containing 5 ring atoms, X is selected from a group consisting of CH₂, CHR⁸ and NR^{7b};
When the ring designated Q¹ is a saturated ring containing 6 ring atoms, X is selected from a group consisting of CH₂, CHR⁸ and NR^{7b};
When the ring designated Q¹ is a saturated ring containing 7 ring atoms, X is selected from a group consisting of CH₂, CHR⁸ and NR^{7b};
R^{1a} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1b} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1c} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1d} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1e} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1b} and R^{1c} are optionally taken together with the atom to which they are bound to form an optionally substituted ring having 5-7 ring atoms optionally containing up to 2 groups selected from oxygen and NH;
R^{1c} and R^{1d} are optionally taken together with the atom to which they are bound to form an optionally substituted ring having 5-7 ring atoms optionally containing up to 2 groups selected from oxygen and NH;
R⁴ is selected from the group consisting of hydrogen, C₁₋₆ linear alkyl, C₃₋₇ branched alkyl,

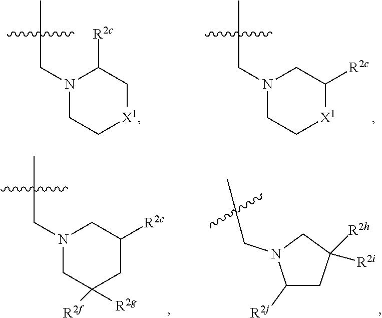

optionally substituted C₃₋₈ cycloalkyl, optionally substituted aryl, and optionally substituted heteroaryl;
X¹ is selected from the group consisting of oxygen, CR^{2a}R^{2b}, C═O, SO₂, and NSO₂R^{2d}.
R^{2a} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2b} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2c} is selected from the group consisting of hydrogen, CO₂R^{2e}, optionally substituted C₁₋₆ linear alkyl, and optionally substitute C₃₋₇ branched alkyl;
R^{2d} is selected from the group consisting of C₁₋₆ linear alkyl and C₃₋₇ branched alkyl;
R^{2e} is selected from the group consisting of hydrogen, C₁₋₆ linear alkyl, and C₃₋₇ branched alkyl;
R^{2f} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2g} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2h} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R²ⁱ is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2j} is selected from the group consisting of hydrogen, CO₂R^{2e}, optionally substituted C₁₋₆ linear alkyl, and optionally substitute C₃₋₇ branched alkyl;
R^{5a} is selected from the group consisting of hydrogen, C₁₋₆ linear alkyl, C₃₋₇ branched alkyl, CONH₂,

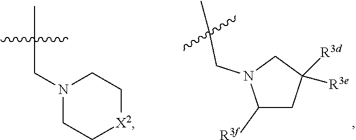

optionally substituted aryl, and optionally substituted heteroaryl;
R^{5b} is selected from a group consisting of hydrogen, C₁₋₆ linear alkyl, and C₃₋₇ branched alkyl;
X² is selected from the group consisting of oxygen, CR^{3a}R^{3b}, C═O, SO₂, NSO₂R^{3c},
R^{3a} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3b} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3c} is selected from the group consisting of C₁₋₆ linear alkyl and C₃₋₇ branched alkyl;
R^{3d} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3e} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3f} is selected from the group consisting of hydrogen, CO₂R^{3g}, optionally substituted C₁₋₆ linear alkyl, and optionally substitute C₃₋₇ branched alkyl;
R^{3g} is selected from the group consisting of hydrogen, C₁₋₆ linear alkyl, and C₃₋₇ branched alkyl;
R⁶ is selected from a group consisting of hydrogen, halogen, optionally substituted C₁₋₆ linear alkyl, optionally substituted C₃₋₇ branched alkyl, optionally substituted C₃₋₈ cycloalkyl, optionally substituted C₁₋₆ linear alkoxy, optionally substituted C₃₋₇ branched alkoxy, optionally substituted C₃₋₈ cycloalkoxy, —OBenzyl,

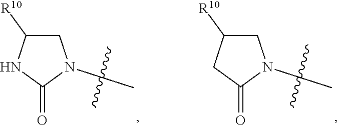

optionally substituted aryl, and optionally substituted heteroaryl;
R⁷ is selected from a group consisting of hydrogen, optionally substituted C₁₋₆ linear alkyl, optionally substituted C₃₋₇ branched alkyl, C₁₋₆ fluoroalkyl, C₃₋₇ branched fluoroalkyl, optionally substituted C₁₋₆ alkenyl, CO₂R¹⁰, CONHR¹⁰, SO₂R¹⁰, and

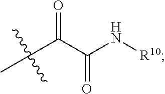

R^{7a} is selected from a group consisting of hydrogen, optionally substituted C₁₋₆ linear alkyl, optionally substituted C₃₋₇ branched alkyl, C₁₋₆ fluoroalkyl, C₃₋₇ branched fluoroalkyl, optionally substituted C₁₋₆ alkenyl, CO₂R¹⁰, CONHR¹⁰, SO₂R¹⁰, and

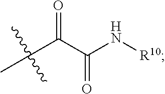

R^{7b} is selected from a group consisting of hydrogen, optionally substituted C₁₋₆ linear alkyl, optionally substituted C₃₋₇ branched alkyl, C₁₋₆ fluoroalkyl, C₃₋₇ branched fluoroalkyl, optionally substituted C₁₋₆ alkenyl, CO₂R¹⁰, CONHR¹⁰, SO₂R¹⁰, and

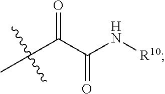

R⁸ is selected from a group consisting of hydrogen, halogen, optionally substituted C₁₋₆ linear alkyl, optionally substituted C₃₋₇ branched alkyl, optionally substituted C₃₋₈ cycloalkyl, optionally substituted aryl, optionally substituted heteroaryl, CO₂R¹⁰, CONHR¹⁰, NHCOR¹⁰, SO₂R¹⁰, and

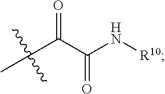

### When R⁸ is

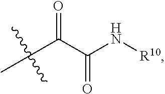

### R⁷ is not

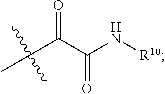

### When R⁸ is

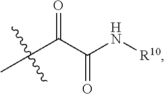

### R^{7a} is not

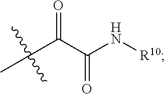

### When R⁸ is

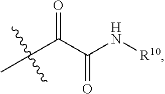

### R^{7b} is not

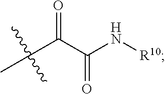

R⁹ is selected from a group consisting of hydrogen, optionally substituted C₁₋₄ alkyl, optionally substituted halo C₁₋₄ alkyl, and optionally substituted C₃₋₇ cycloalkyl;
R¹⁰ is selected from a group consisting of hydrogen, optionally substituted C₁₋₆ linear alkyl, optionally substituted C₃₋₇ branched alkyl, optionally substituted C₃₋₈ cycloalkyl, benzyl, optionally substituted aryl, and optionally substituted heteroaryl;
R¹¹ is selected from a group consisting of hydrogen, optionally substituted C₁₋₆ linear alkyl, optionally substituted C₃₋₇ branched alkyl, optionally substituted C₃₋₈ cycloalkyl, optionally substituted aryl, and optionally substituted heteroaryl.
The compounds of the present invention include compounds having formula (II):

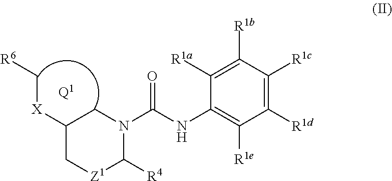

Including enantiomers, diasteromers, hydrates, solvates, pharmaceutically acceptable salts, prodrugs and complexes thereof.
The compounds of the present invention include compounds having formula (IIa):

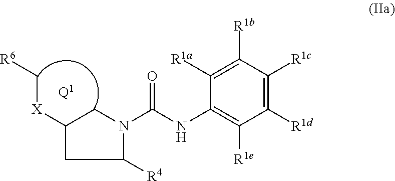

Including enantiomers, diasteromers, hydrates, solvates, pharmaceutically acceptable salts, prodrugs and complexes thereof.
The compounds of the present invention include compounds having formula (IIb):

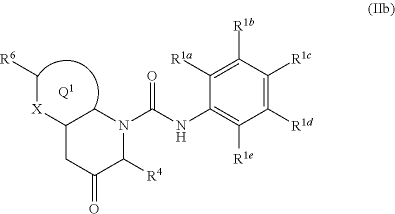

Including enantiomers, diasteromers, hydrates, solvates, pharmaceutically acceptable salts, prodrugs and complexes thereof.
The compounds of the present invention include compounds having formula (IIc):

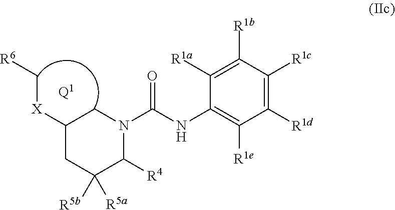

Including enantiomers, diasteromers, hydrates, solvates, pharmaceutically acceptable salts, prodrugs and complexes thereof.
The compounds of the present invention include compounds having formula (IId):

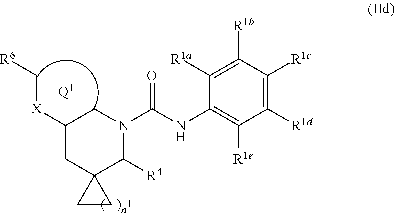

Including enantiomers, diasteromers, hydrates, solvates, pharmaceutically acceptable salts, prodrugs and complexes thereof.
The compounds of the present invention include compounds having formula (IIe):

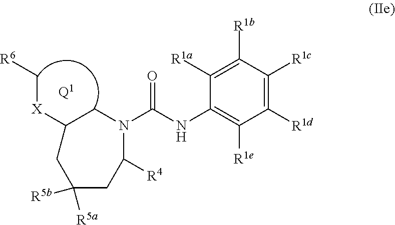

Including enantiomers, diasteromers, hydrates, solvates, pharmaceutically acceptable salts, prodrugs and complexes thereof.
The compounds of the present invention include compounds having formula (IIf):

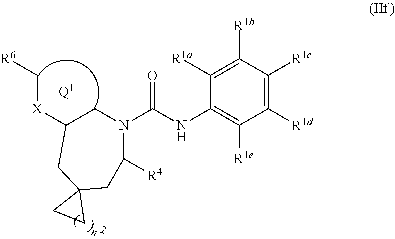

Including enantiomers, diasteromers, hydrates, solvates, pharmaceutically acceptable salts, prodrugs and complexes thereof.
The compounds of the present invention include compounds having formula (III):

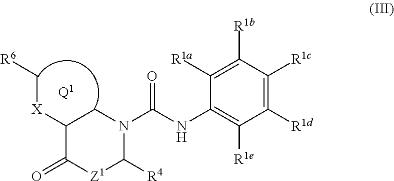

Including enantiomers, diasteromers, hydrates, solvates, pharmaceutically acceptable salts, prodrugs and complexes thereof.
The compounds of the present invention include compounds having formula (IIIa):

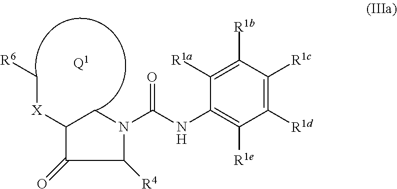

Including enantiomers, diasteromers, hydrates, solvates, pharmaceutically acceptable salts, prodrugs and complexes thereof.
The compounds of the present invention include compounds having formula (IIIb):

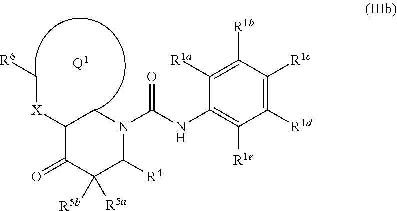

Including enantiomers, diasteromers, hydrates, solvates, pharmaceutically acceptable salts, prodrugs and complexes thereof.
The compounds of the present invention include compounds having formula (IIIc):

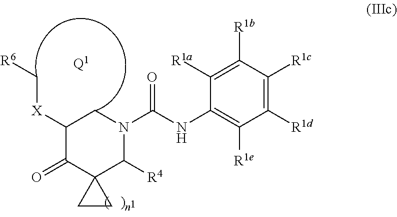

Including enantiomers, diasteromers, hydrates, solvates, pharmaceutically acceptable salts, prodrugs and complexes thereof.
The compounds of the present invention include compounds having formula (IIId):

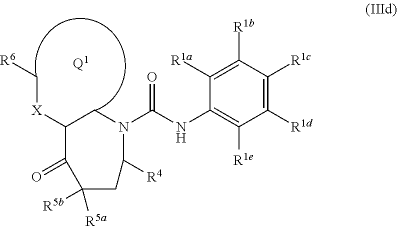

Including enantiomers, diasteromers, hydrates, solvates, pharmaceutically acceptable salts, prodrugs and complexes thereof.
The compounds of the present invention include compounds having formula (IIIe):

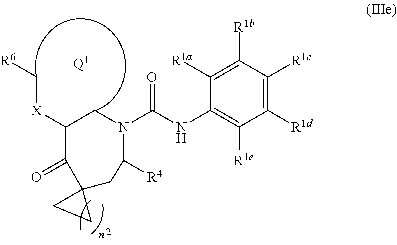

Including enantiomers, diasteromers, hydrates, solvates, pharmaceutically acceptable salts, prodrugs and complexes thereof.
The compounds of the present invention include compounds having formula (IV):

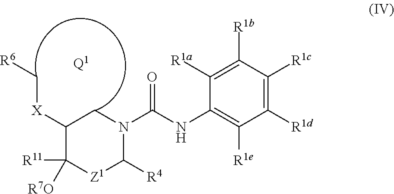

Including enantiomers, diasteromers, hydrates, solvates, pharmaceutically acceptable salts, prodrugs and complexes thereof.
The compounds of the present invention include compounds having formula (IVa):

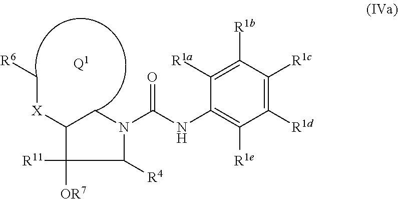

Including enantiomers, diasteromers, hydrates, solvates, pharmaceutically acceptable salts, prodrugs and complexes thereof.
The compounds of the present invention include compounds having formula (IVb):

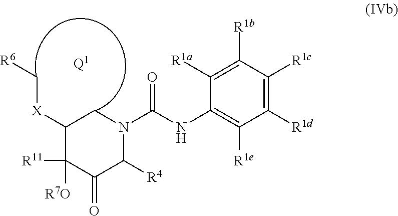

Including enantiomers, diasteromers, hydrates, solvates, pharmaceutically acceptable salts, prodrugs and complexes thereof.
The compounds of the present invention include compounds having formula (IVc):

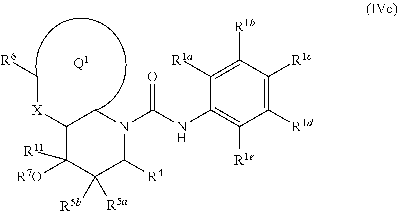

Including enantiomers, diasteromers, hydrates, solvates, pharmaceutically acceptable salts, prodrugs and complexes thereof.
The compounds of the present invention include compounds having formula (IVd):

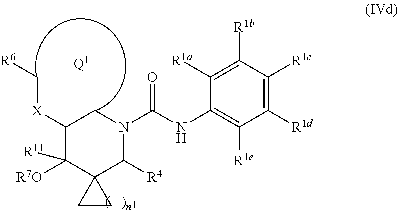

Including enantiomers, diasteromers, hydrates, solvates, pharmaceutically acceptable salts, prodrugs and complexes thereof.
The compounds of the present invention include compounds having formula (IVe):

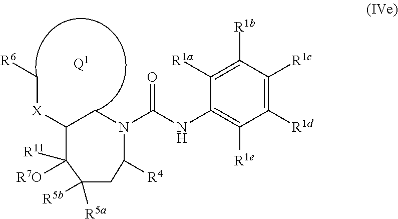

Including enantiomers, diasteromers, hydrates, solvates, pharmaceutically acceptable salts, prodrugs and complexes thereof.
The compounds of the present invention include compounds having formula (IVf):

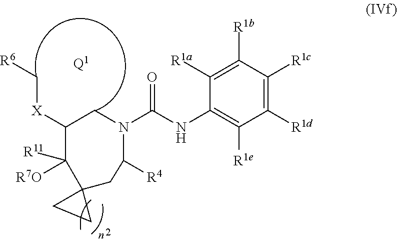

Including enantiomers, diasteromers, hydrates, solvates, pharmaceutically acceptable salts, prodrugs and complexes thereof.
The compounds of the present invention include compounds having formula (V):

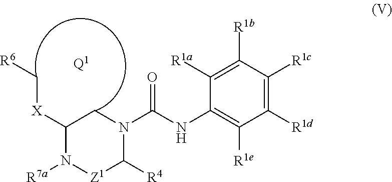

Including enantiomers, diasteromers, hydrates, solvates, pharmaceutically acceptable salts, prodrugs and complexes thereof.
The compounds of the present invention include compounds having formula (Va):

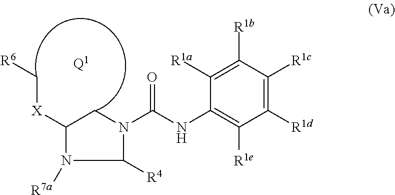

Including enantiomers, diasteromers, hydrates, solvates, pharmaceutically acceptable salts, prodrugs and complexes thereof.
The compounds of the present invention include compounds having formula (Vb):

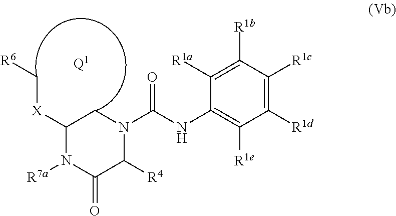

Including enantiomers, diasteromers, hydrates, solvates, pharmaceutically acceptable salts, prodrugs and complexes thereof.
The compounds of the present invention include compounds having formula (Vc):

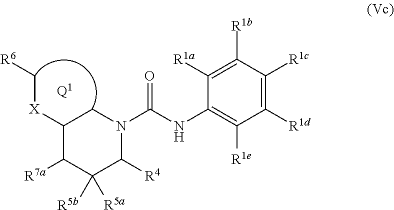

Including enantiomers, diasteromers, hydrates, solvates, pharmaceutically acceptable salts, prodrugs and complexes thereof.
The compounds of the present invention include compounds having formula (Vd):

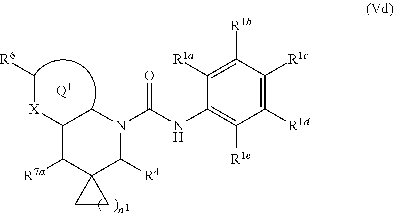

Including enantiomers, diasteromers, hydrates, solvates, pharmaceutically acceptable salts, prodrugs and complexes thereof.
The compounds of the present invention include compounds having formula (Ve):

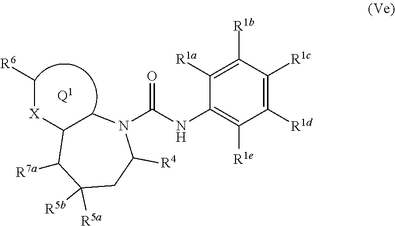

Including enantiomers, diasteromers, hydrates, solvates, pharmaceutically acceptable salts, prodrugs and complexes thereof.
The compounds of the present invention include compounds having formula (Vf):

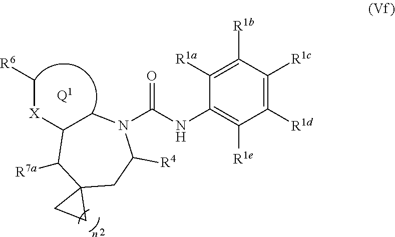

Including enantiomers, diasteromers, hydrates, solvates, pharmaceutically acceptable salts, prodrugs and complexes thereof.
The compounds of the present invention include compounds having formula (VI):

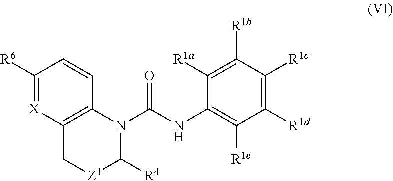

Including enantiomers, diasteromers, hydrates, solvates, pharmaceutically acceptable salts, prodrugs and complexes thereof.
The compounds of the present invention include compounds having formula (VIa):

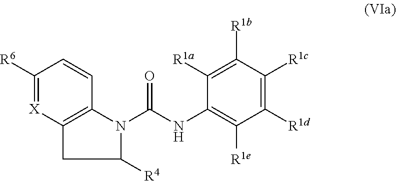

Including enantiomers, diasteromers, hydrates, solvates, pharmaceutically acceptable salts, prodrugs and complexes thereof.
The compounds of the present invention include compounds having formula (VIb):

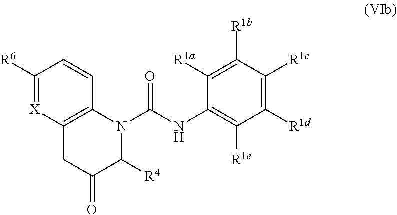

Including enantiomers, diasteromers, hydrates, solvates, pharmaceutically acceptable salts, prodrugs and complexes thereof.
The compounds of the present invention include compounds having formula (VIc):

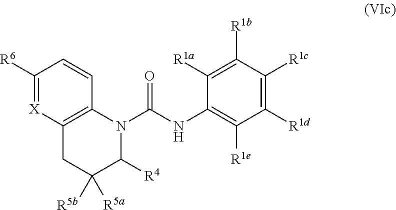

Including enantiomers, diasteromers, hydrates, solvates, pharmaceutically acceptable salts, prodrugs and complexes thereof.
The compounds of the present invention include compounds having formula (VId):

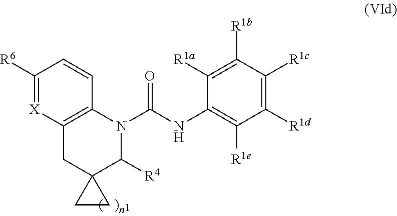

Including enantiomers, diasteromers, hydrates, solvates, pharmaceutically acceptable salts, prodrugs and complexes thereof.
The compounds of the present invention include compounds having formula (VIe):

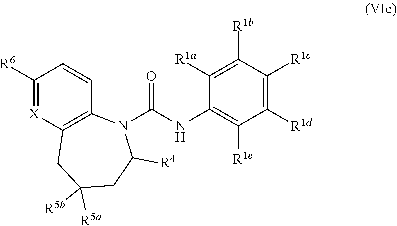

Including enantiomers, diasteromers, hydrates, solvates, pharmaceutically acceptable salts, prodrugs and complexes thereof.
The compounds of the present invention include compounds having formula (VIf):

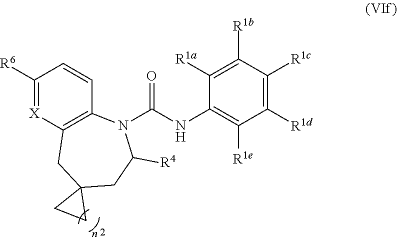

Including enantiomers, diasteromers, hydrates, solvates, pharmaceutically acceptable salts, prodrugs and complexes thereof.
The compounds of the present invention include compounds having formula (VII):

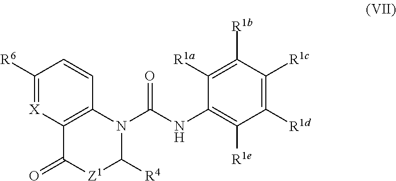

Including enantiomers, diasteromers, hydrates, solvates, pharmaceutically acceptable salts, prodrugs and complexes thereof.
The compounds of the present invention include compounds having formula (VIIa):

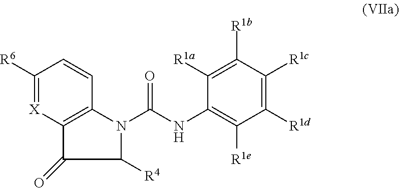

Including enantiomers, diasteromers, hydrates, solvates, pharmaceutically acceptable salts, prodrugs and complexes thereof.
The compounds of the present invention include compounds having formula (VIIb):

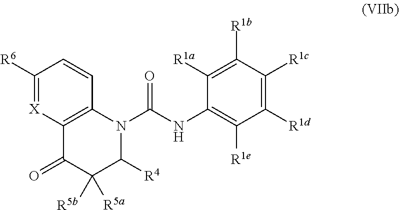

Including enantiomers, diasteromers, hydrates, solvates, pharmaceutically acceptable salts, prodrugs and complexes thereof.
The compounds of the present invention include compounds having formula (VIIc):

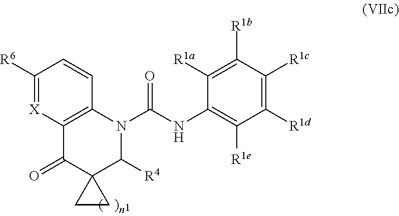

Including enantiomers, diasteromers, hydrates, solvates, pharmaceutically acceptable salts, prodrugs and complexes thereof.
The compounds of the present invention include compounds having formula (VIId):

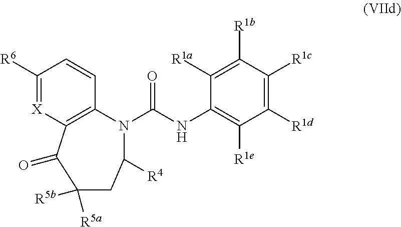

Including enantiomers, diasteromers, hydrates, solvates, pharmaceutically acceptable salts, prodrugs and complexes thereof.
The compounds of the present invention include compounds having formula (VIIe):

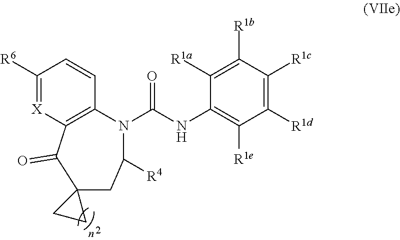

Including enantiomers, diasteromers, hydrates, solvates, pharmaceutically acceptable salts, prodrugs and complexes thereof.
The compounds of the present invention include compounds having formula (VIII):

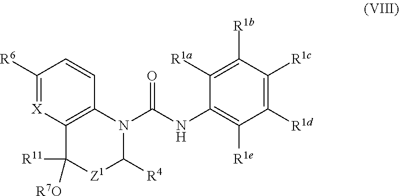

Including enantiomers, diasteromers, hydrates, solvates, pharmaceutically acceptable salts, prodrugs and complexes thereof.
The compounds of the present invention include compounds having formula (VIIIa):

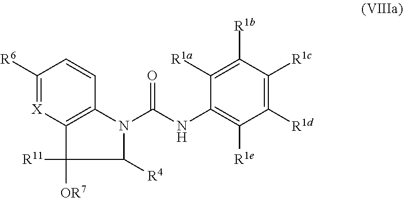

Including enantiomers, diasteromers, hydrates, solvates, pharmaceutically acceptable salts, prodrugs and complexes thereof.
The compounds of the present invention include compounds having formula (VIIIb):

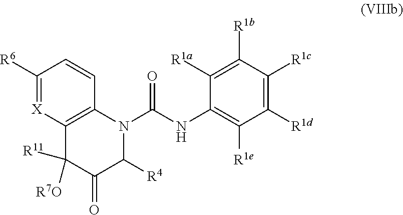

Including enantiomers, diasteromers, hydrates, solvates, pharmaceutically acceptable salts, prodrugs and complexes thereof.
The compounds of the present invention include compounds having formula (VIIIc):

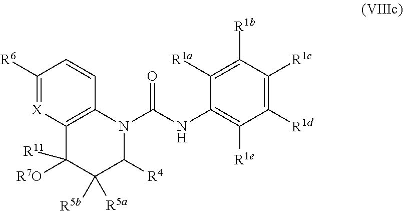

Including enantiomers, diasteromers, hydrates, solvates, pharmaceutically acceptable salts, prodrugs and complexes thereof.
The compounds of the present invention include compounds having formula (VIIId):

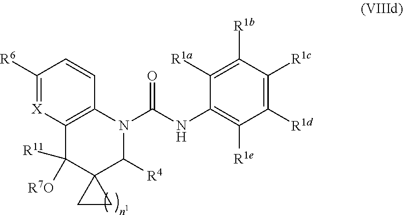

Including enantiomers, diasteromers, hydrates, solvates, pharmaceutically acceptable salts, prodrugs and complexes thereof.
The compounds of the present invention include compounds having formula (VIIIe):

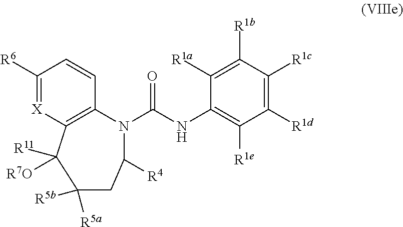

Including enantiomers, diasteromers, hydrates, solvates, pharmaceutically acceptable salts, prodrugs and complexes thereof.
The compounds of the present invention include compounds having formula (VIIIf):

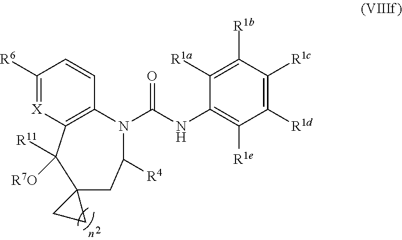

Including enantiomers, diasteromers, hydrates, solvates, pharmaceutically acceptable salts, prodrugs and complexes thereof.
The compounds of the present invention include compounds having formula (IX):

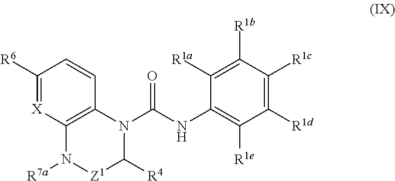

Including enantiomers, diasteromers, hydrates, solvates, pharmaceutically acceptable salts, prodrugs and complexes thereof.
The compounds of the present invention include compounds having formula (IXa):

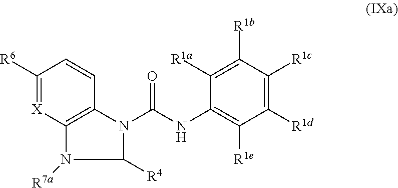

Including enantiomers, diasteromers, hydrates, solvates, pharmaceutically acceptable salts, prodrugs and complexes thereof.
The compounds of the present invention include compounds having formula (IXb):

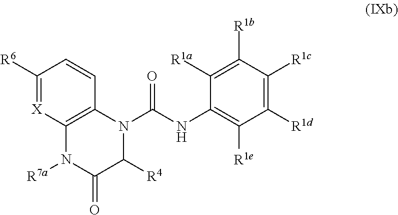

Including enantiomers, diasteromers, hydrates, solvates, pharmaceutically acceptable salts, prodrugs and complexes thereof.
The compounds of the present invention include compounds having formula (IXc):

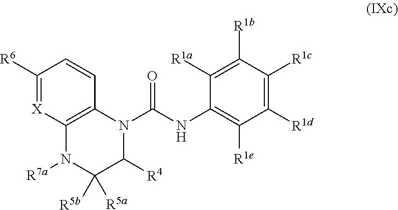

Including enantiomers, diasteromers, hydrates, solvates, pharmaceutically acceptable salts, prodrugs and complexes thereof.
The compounds of the present invention include compounds having formula (IXd):

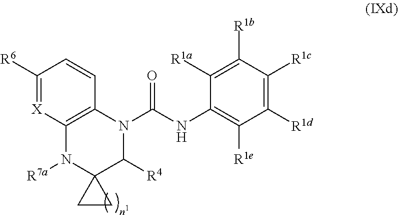

Including enantiomers, diasteromers, hydrates, solvates, pharmaceutically acceptable salts, prodrugs and complexes thereof.
The compounds of the present invention include compounds having formula (IXe):

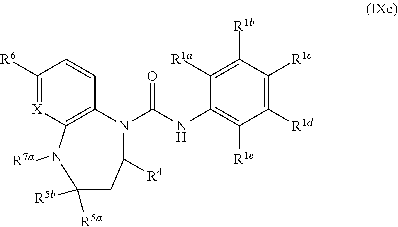

Including enantiomers, diasteromers, hydrates, solvates, pharmaceutically acceptable salts, prodrugs and complexes thereof.
The compounds of the present invention include compounds having formula (IXf):

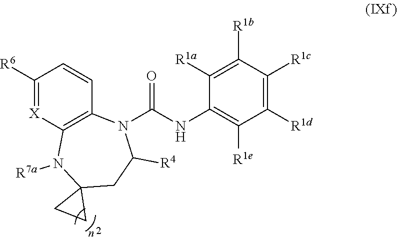

Including enantiomers, diasteromers, hydrates, solvates, pharmaceutically acceptable salts, prodrugs and complexes thereof.
The compounds of the present invention include compounds having formula (X):

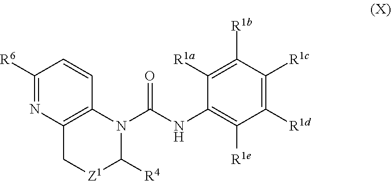

Including enantiomers, diasteromers, hydrates, solvates, pharmaceutically acceptable salts, prodrugs and complexes thereof.
The compounds of the present invention include compounds having formula (Xa):

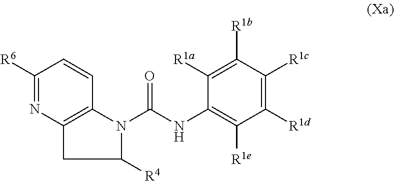

Including enantiomers, diasteromers, hydrates, solvates, pharmaceutically acceptable salts, prodrugs and complexes thereof.
The compounds of the present invention include compounds having formula (Xb):

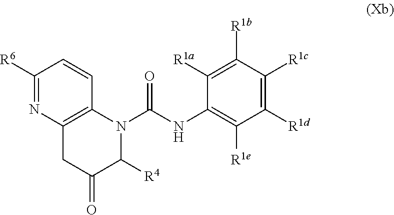

Including enantiomers, diasteromers, hydrates, solvates, pharmaceutically acceptable salts, prodrugs and complexes thereof.
The compounds of the present invention include compounds having formula (Xc):

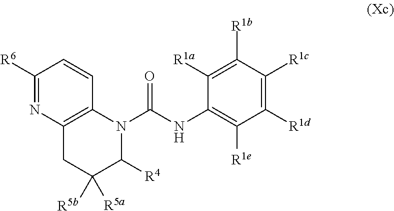

Including enantiomers, diasteromers, hydrates, solvates, pharmaceutically acceptable salts, prodrugs and complexes thereof.
The compounds of the present invention include compounds having formula (Xd):

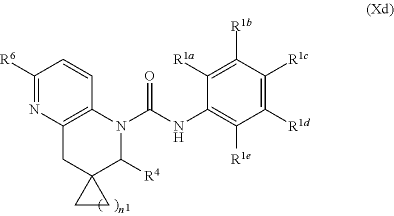

Including enantiomers, diasteromers, hydrates, solvates, pharmaceutically acceptable salts, prodrugs and complexes thereof.
The compounds of the present invention include compounds having formula (Xe):

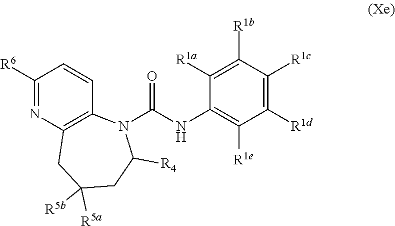

Including enantiomers, diasteromers, hydrates, solvates, pharmaceutically acceptable salts, prodrugs and complexes thereof.
The compounds of the present invention include compounds having formula (Xf):

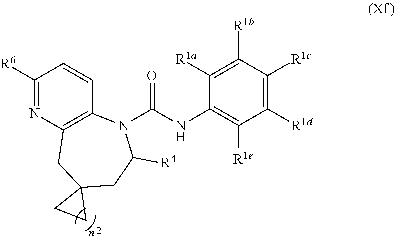

Including enantiomers, diasteromers, hydrates, solvates, pharmaceutically acceptable salts, prodrugs and complexes thereof.
The compounds of the present invention include compounds having formula (XI):

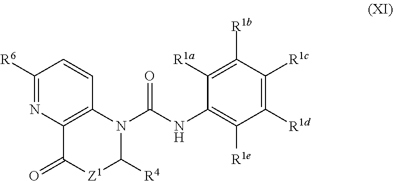

Including enantiomers, diasteromers, hydrates, solvates, pharmaceutically acceptable salts, prodrugs and complexes thereof.
The compounds of the present invention include compounds having formula (XIa):

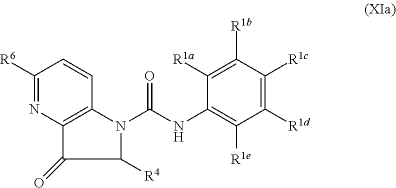

Including enantiomers, diasteromers, hydrates, solvates, pharmaceutically acceptable salts, prodrugs and complexes thereof.
The compounds of the present invention include compounds having formula (XIb):

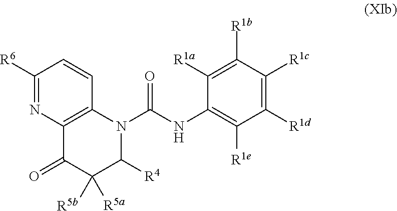

Including enantiomers, diasteromers, hydrates, solvates, pharmaceutically acceptable salts, prodrugs and complexes thereof.
The compounds of the present invention include compounds having formula (XIc):

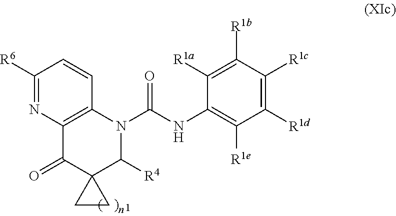

Including enantiomers, diasteromers, hydrates, solvates, pharmaceutically acceptable salts, prodrugs and complexes thereof.
The compounds of the present invention include compounds having formula (XId):

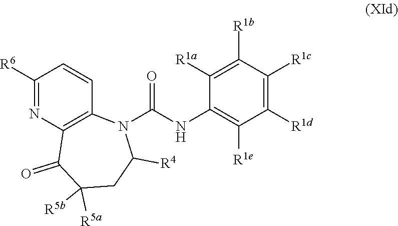

Including enantiomers, diasteromers, hydrates, solvates, pharmaceutically acceptable salts, prodrugs and complexes thereof.
The compounds of the present invention include compounds having formula (XIe):

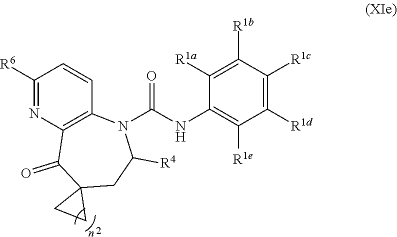

Including enantiomers, diasteromers, hydrates, solvates, pharmaceutically acceptable salts, prodrugs and complexes thereof.
The compounds of the present invention include compounds having formula (XII):

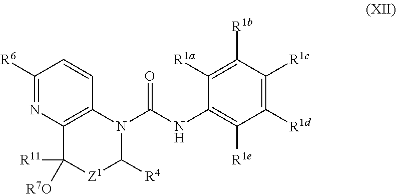

Including enantiomers, diasteromers, hydrates, solvates, pharmaceutically acceptable salts, prodrugs and complexes thereof.
The compounds of the present invention include compounds having formula (XIIa):

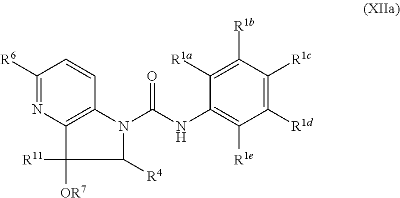

Including enantiomers, diasteromers, hydrates, solvates, pharmaceutically acceptable salts, prodrugs and complexes thereof.
The compounds of the present invention include compounds having formula (XIIb):

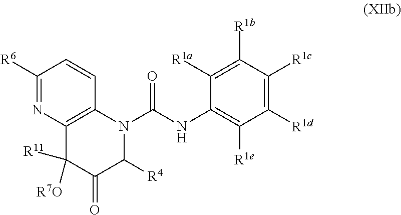

Including enantiomers, diasteromers, hydrates, solvates, pharmaceutically acceptable salts, prodrugs and complexes thereof.
The compounds of the present invention include compounds having formula (XIIc):

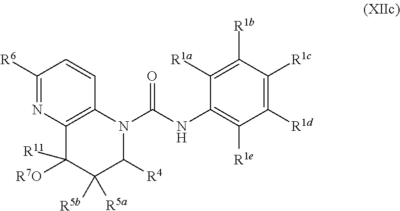

Including enantiomers, diasteromers, hydrates, solvates, pharmaceutically acceptable salts, prodrugs and complexes thereof.
The compounds of the present invention include compounds having formula (XIId):

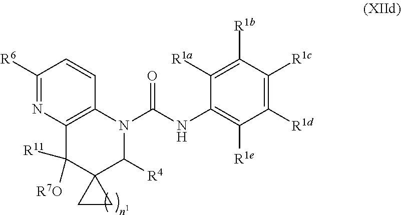

Including enantiomers, diasteromers, hydrates, solvates, pharmaceutically acceptable salts, prodrugs and complexes thereof.
The compounds of the present invention include compounds having formula (XIIe):

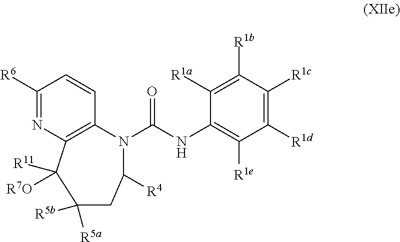

Including enantiomers, diasteromers, hydrates, solvates, pharmaceutically acceptable salts, prodrugs and complexes thereof.
The compounds of the present invention include compounds having formula (XIIf):

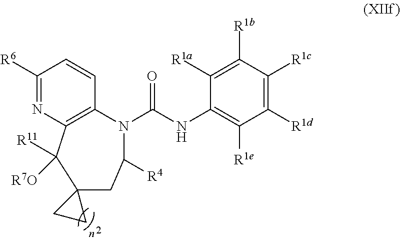

Including enantiomers, diasteromers, hydrates, solvates, pharmaceutically acceptable salts, prodrugs and complexes thereof
The compounds of the present invention include compounds having formula (XIII):

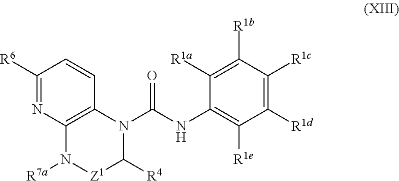

Including enantiomers, diasteromers, hydrates, solvates, pharmaceutically acceptable salts, prodrugs and complexes thereof.
The compounds of the present invention include compounds having formula (XIIIa):

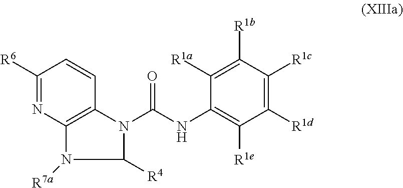

Including enantiomers, diasteromers, hydrates, solvates, pharmaceutically acceptable salts, prodrugs and complexes thereof.
The compounds of the present invention include compounds having formula (XIIIb):

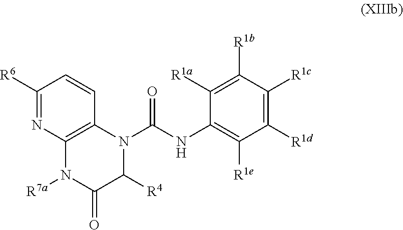

Including enantiomers, diasteromers, hydrates, solvates, pharmaceutically acceptable salts, prodrugs and complexes thereof.
The compounds of the present invention include compounds having formula (XIIIc):

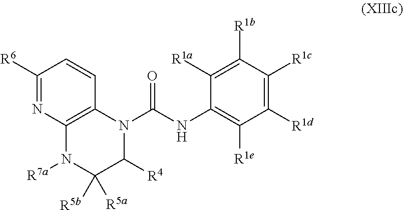

Including enantiomers, diasteromers, hydrates, solvates, pharmaceutically acceptable salts, prodrugs and complexes thereof.
The compounds of the present invention include compounds having formula (XIIId):

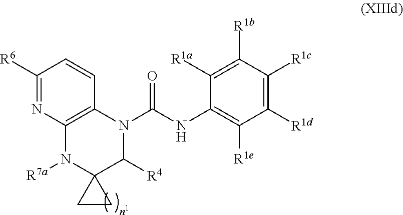

Including enantiomers, diasteromers, hydrates, solvates, pharmaceutically acceptable salts, prodrugs and complexes thereof.
The compounds of the present invention include compounds having formula (XIIIe):

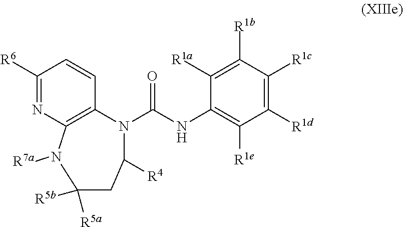

Including enantiomers, diasteromers, hydrates, solvates, pharmaceutically acceptable salts, prodrugs and complexes thereof.
The compounds of the present invention include compounds having formula (XIIIf):

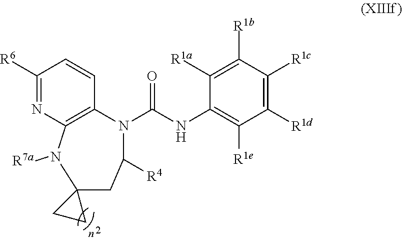

Including enantiomers, diasteromers, hydrates, solvates, pharmaceutically acceptable salts, prodrugs and complexes thereof.
The compounds of the present invention include compounds having formula (XIV):

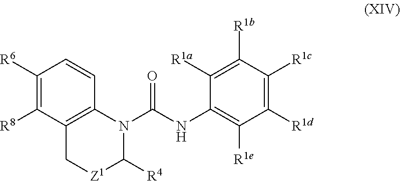

Including enantiomers, diasteromers, hydrates, solvates, pharmaceutically acceptable salts, prodrugs and complexes thereof.
The compounds of the present invention include compounds having formula (XIVa):

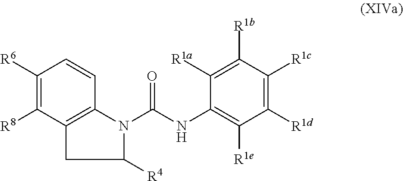

Including enantiomers, diasteromers, hydrates, solvates, pharmaceutically acceptable salts, prodrugs and complexes thereof.
The compounds of the present invention include compounds having formula (XIVb):

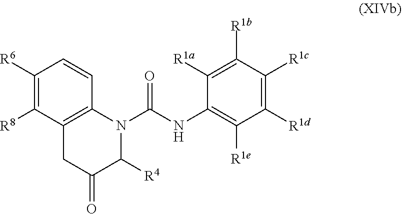

Including enantiomers, diasteromers, hydrates, solvates, pharmaceutically acceptable salts, prodrugs and complexes thereof.
The compounds of the present invention include compounds having formula (XIVc):

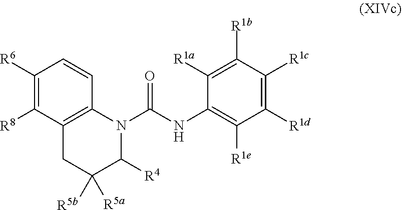

Including enantiomers, diasteromers, hydrates, solvates, pharmaceutically acceptable salts, prodrugs and complexes thereof.
The compounds of the present invention include compounds having formula (XIVd):

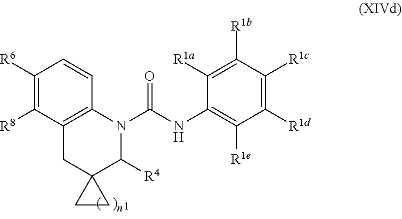

Including enantiomers, diasteromers, hydrates, solvates, pharmaceutically acceptable salts, prodrugs and complexes thereof.
The compounds of the present invention include compounds having formula (XIVe):

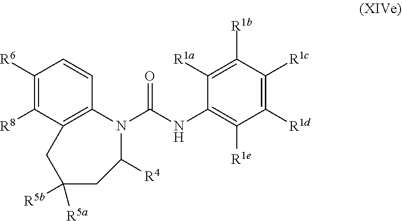

Including enantiomers, diasteromers, hydrates, solvates, pharmaceutically acceptable salts, prodrugs and complexes thereof.
The compounds of the present invention include compounds having formula (XIVf):

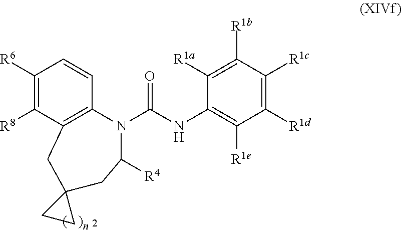

Including enantiomers, diasteromers, hydrates, solvates, pharmaceutically acceptable salts, prodrugs and complexes thereof.
The compounds of the present invention include compounds having formula (XV):

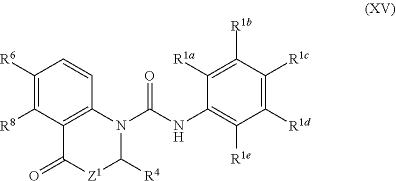

Including enantiomers, diasteromers, hydrates, solvates, pharmaceutically acceptable salts, prodrugs and complexes thereof.
The compounds of the present invention include compounds having formula (XVa):

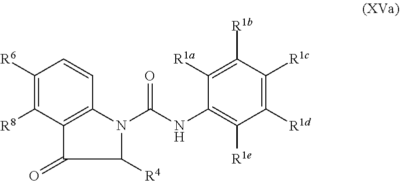

Including enantiomers, diasteromers, hydrates, solvates, pharmaceutically acceptable salts, prodrugs and complexes thereof.
The compounds of the present invention include compounds having formula (XVb):

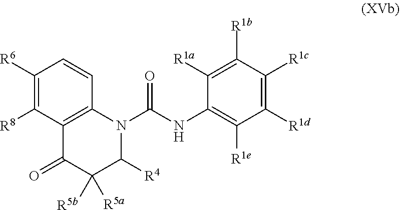

Including enantiomers, diasteromers, hydrates, solvates, pharmaceutically acceptable salts, prodrugs and complexes thereof.
The compounds of the present invention include compounds having formula (XVc):

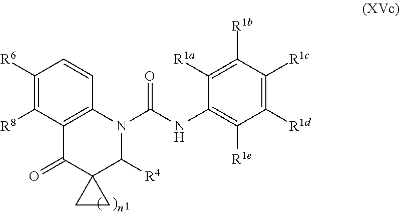

Including enantiomers, diasteromers, hydrates, solvates, pharmaceutically acceptable salts, prodrugs and complexes thereof.
The compounds of the present invention include compounds having formula (XVd):

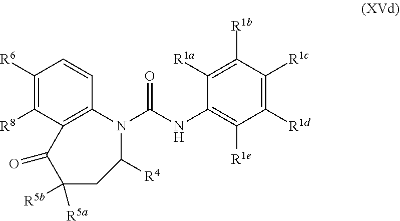

Including enantiomers, diasteromers, hydrates, solvates, pharmaceutically acceptable salts, prodrugs and complexes thereof.
The compounds of the present invention include compounds having formula (XVe):

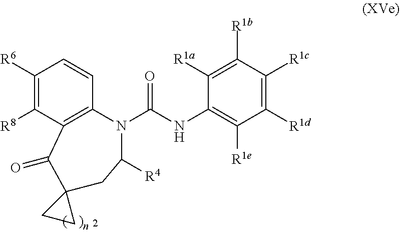

Including enantiomers, diasteromers, hydrates, solvates, pharmaceutically acceptable salts, prodrugs and complexes thereof.
The compounds of the present invention include compounds having formula (XVI):

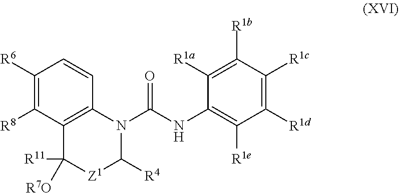

Including enantiomers, diasteromers, hydrates, solvates, pharmaceutically acceptable salts, prodrugs and complexes thereof.
The compounds of the present invention include compounds having formula (XVIa):

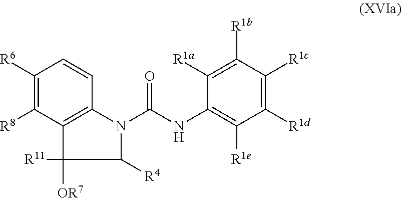

Including enantiomers, diasteromers, hydrates, solvates, pharmaceutically acceptable salts, prodrugs and complexes thereof.
The compounds of the present invention include compounds having formula (XVIb):

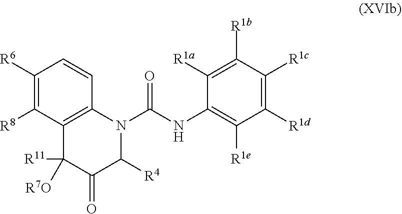

Including enantiomers, diasteromers, hydrates, solvates, pharmaceutically acceptable salts, prodrugs and complexes thereof.
The compounds of the present invention include compounds having formula (XVIc):

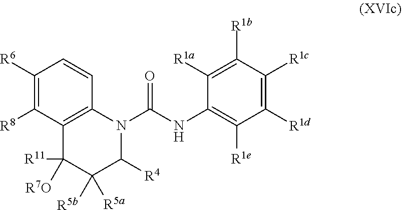

Including enantiomers, diasteromers, hydrates, solvates, pharmaceutically acceptable salts, prodrugs and complexes thereof.
The compounds of the present invention include compounds having formula (XVId):

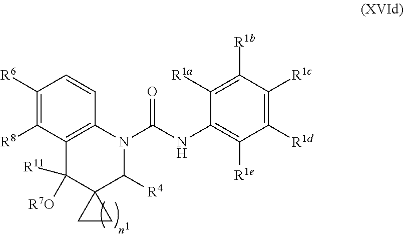

Including enantiomers, diasteromers, hydrates, solvates, pharmaceutically acceptable salts, prodrugs and complexes thereof.
The compounds of the present invention include compounds having formula (XVIe):

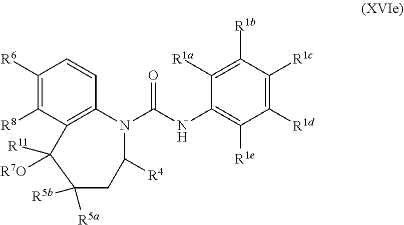

Including enantiomers, diasteromers, hydrates, solvates, pharmaceutically acceptable salts, prodrugs and complexes thereof.
The compounds of the present invention include compounds having formula (XVIf):

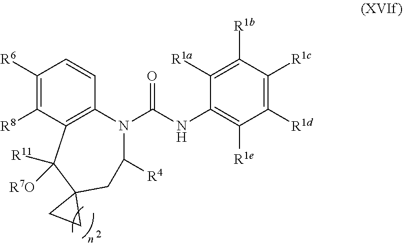

Including enantiomers, diasteromers, hydrates, solvates, pharmaceutically acceptable salts, prodrugs and complexes thereof.
The compounds of the present invention include compounds having formula (XVII):

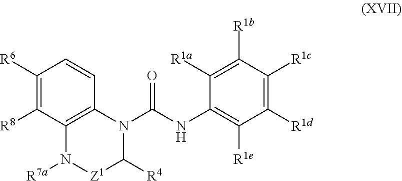

Including enantiomers, diasteromers, hydrates, solvates, pharmaceutically acceptable salts, prodrugs and complexes thereof.
The compounds of the present invention include compounds having formula (XVIIa):

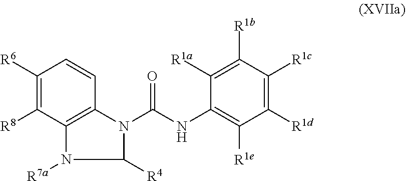

Including enantiomers, diasteromers, hydrates, solvates, pharmaceutically acceptable salts, prodrugs and complexes thereof.
The compounds of the present invention include compounds having formula (XVIIb):

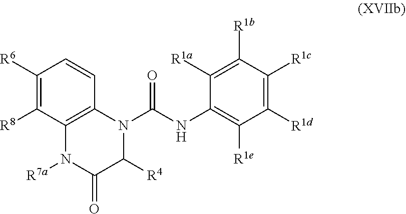

Including enantiomers, diasteromers, hydrates, solvates, pharmaceutically acceptable salts, prodrugs and complexes thereof.
The compounds of the present invention include compounds having formula (XVIIc):

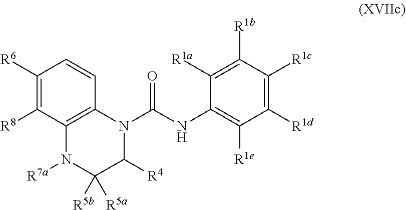

Including enantiomers, diasteromers, hydrates, solvates, pharmaceutically acceptable salts, prodrugs and complexes thereof.
The compounds of the present invention include compounds having formula (XVIId):

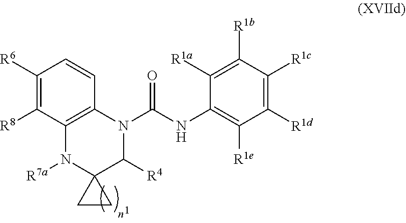

Including enantiomers, diasteromers, hydrates, solvates, pharmaceutically acceptable salts, prodrugs and complexes thereof.
The compounds of the present invention include compounds having formula (XVIIe):

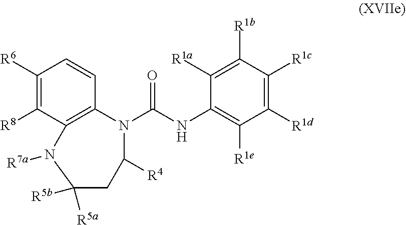

Including enantiomers, diasteromers, hydrates, solvates, pharmaceutically acceptable salts, prodrugs and complexes thereof.
The compounds of the present invention include compounds having formula (XVIIf):

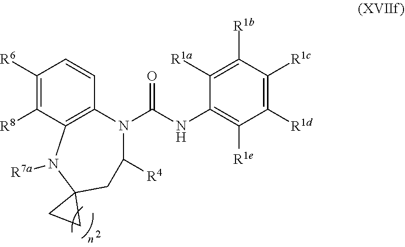

Including enantiomers, diasteromers, hydrates, solvates, pharmaceutically acceptable salts, prodrugs and complexes thereof.
The compounds of the present invention include compounds having formula (XVIIII):

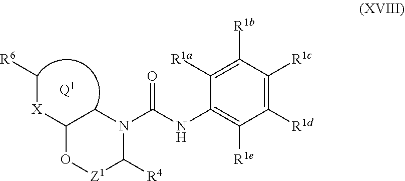

Including enantiomers, diasteromers, hydrates, solvates, pharmaceutically acceptable salts, prodrugs and complexes thereof.
The compounds of the present invention include compounds having formula (XVIIIa):

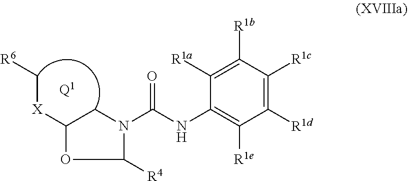

Including enantiomers, diasteromers, hydrates, solvates, pharmaceutically acceptable salts, prodrugs and complexes thereof.
The compounds of the present invention include compounds having formula (XVIIIb):

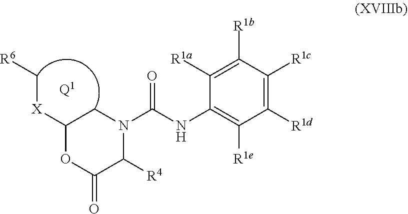

Including enantiomers, diasteromers, hydrates, solvates, pharmaceutically acceptable salts, prodrugs and complexes thereof.
The compounds of the present invention include compounds having formula (XVIIIc):

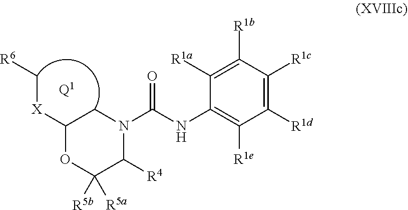

Including enantiomers, diasteromers, hydrates, solvates, pharmaceutically acceptable salts, prodrugs and complexes thereof.
The compounds of the present invention include compounds having formula (XVIIId):

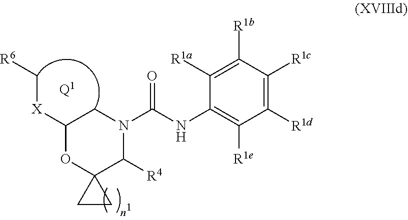

Including enantiomers, diasteromers, hydrates, solvates, pharmaceutically acceptable salts, prodrugs and complexes thereof.
The compounds of the present invention include compounds having formula (XVIIIe):

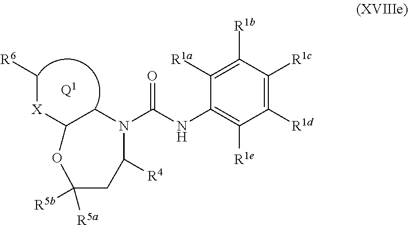

Including enantiomers, diasteromers, hydrates, solvates, pharmaceutically acceptable salts, prodrugs and complexes thereof.
The compounds of the present invention include compounds having formula (XVIIIf):

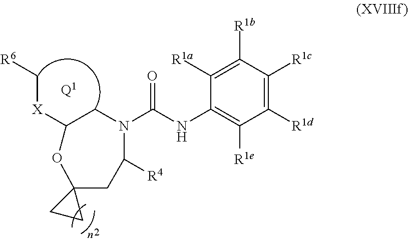

Including enantiomers, diasteromers, hydrates, solvates, pharmaceutically acceptable salts, prodrugs and complexes thereof.
The compounds of the present invention include compounds having formula (VI):

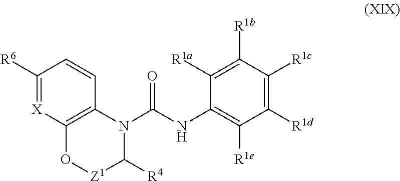

Including enantiomers, diasteromers, hydrates, solvates, pharmaceutically acceptable salts, prodrugs and complexes thereof.
The compounds of the present invention include compounds having formula (XIXa):

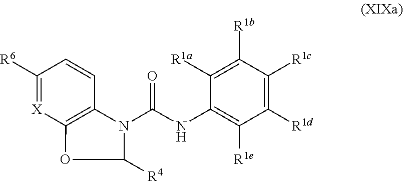

Including enantiomers, diasteromers, hydrates, solvates, pharmaceutically acceptable salts, prodrugs and complexes thereof.
The compounds of the present invention include compounds having formula (XIXb):

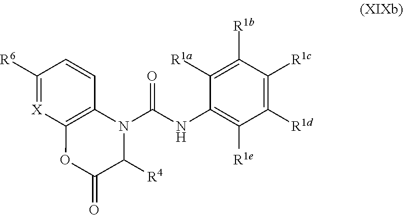

Including enantiomers, diasteromers, hydrates, solvates, pharmaceutically acceptable salts, prodrugs and complexes thereof.
The compounds of the present invention include compounds having formula (XIXc):

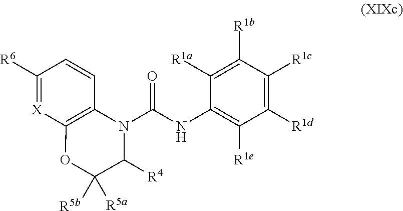

Including enantiomers, diasteromers, hydrates, solvates, pharmaceutically acceptable salts, prodrugs and complexes thereof.
The compounds of the present invention include compounds having formula (XIXd):

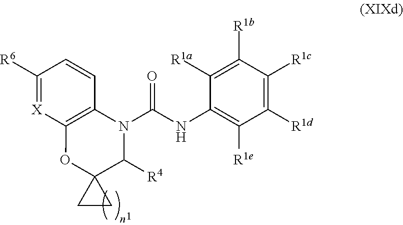

Including enantiomers, diasteromers, hydrates, solvates, pharmaceutically acceptable salts, prodrugs and complexes thereof.
The compounds of the present invention include compounds having formula (XIXe):

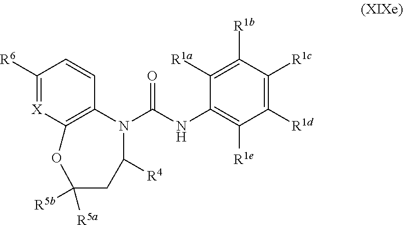

Including enantiomers, diasteromers, hydrates, solvates, pharmaceutically acceptable salts, prodrugs and complexes thereof.
The compounds of the present invention include compounds having formula (XIXf):

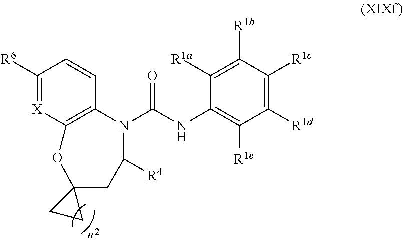

Including enantiomers, diasteromers, hydrates, solvates, pharmaceutically acceptable salts, prodrugs and complexes thereof.
The compounds of the present invention include compounds having formula (XX):

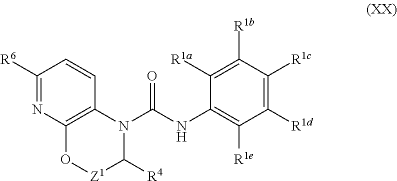

Including enantiomers, diasteromers, hydrates, solvates, pharmaceutically acceptable salts, prodrugs and complexes thereof.
The compounds of the present invention include compounds having formula (XXa):

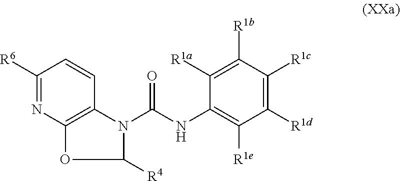

Including enantiomers, diasteromers, hydrates, solvates, pharmaceutically acceptable salts, prodrugs and complexes thereof.
The compounds of the present invention include compounds having formula (XXb):

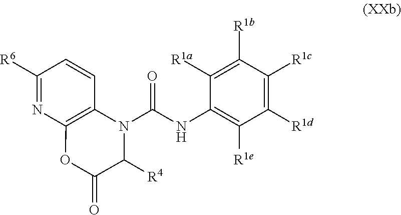

Including enantiomers, diasteromers, hydrates, solvates, pharmaceutically acceptable salts, prodrugs and complexes thereof.
The compounds of the present invention include compounds having formula (XXc):

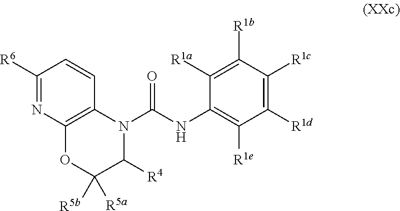

Including enantiomers, diasteromers, hydrates, solvates, pharmaceutically acceptable salts, prodrugs and complexes thereof.
The compounds of the present invention include compounds having formula (XXd):

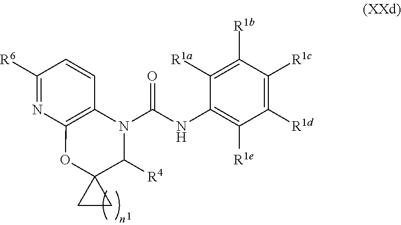

Including enantiomers, diasteromers, hydrates, solvates, pharmaceutically acceptable salts, prodrugs and complexes thereof.
The compounds of the present invention include compounds having formula (XXe):

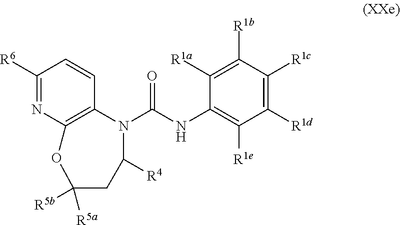

Including enantiomers, diasteromers, hydrates, solvates, pharmaceutically acceptable salts, prodrugs and complexes thereof.
The compounds of the present invention include compounds having formula (XXf):

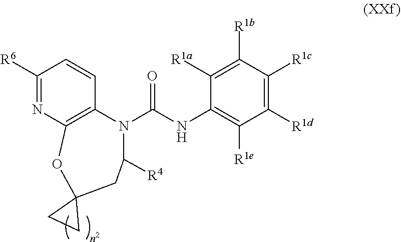

Including enantiomers, diasteromers, hydrates, solvates, pharmaceutically acceptable salts, prodrugs and complexes thereof.
The compounds of the present invention include compounds having formula (XXI):

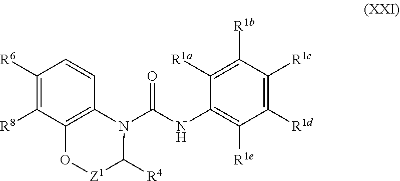

Including enantiomers, diasteromers, hydrates, solvates, pharmaceutically acceptable salts, prodrugs and complexes thereof.
The compounds of the present invention include compounds having formula (XXIa):

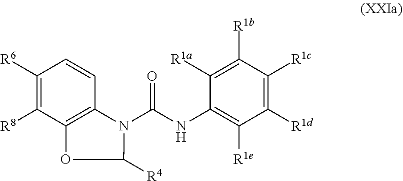

Including enantiomers, diasteromers, hydrates, solvates, pharmaceutically acceptable salts, prodrugs and complexes thereof.
The compounds of the present invention include compounds having formula (XXIb):

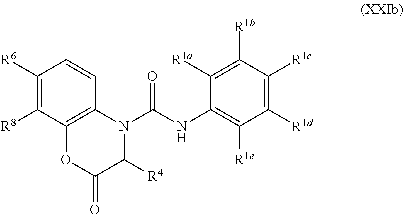

Including enantiomers, diasteromers, hydrates, solvates, pharmaceutically acceptable salts, prodrugs and complexes thereof.
The compounds of the present invention include compounds having formula (XXIc):

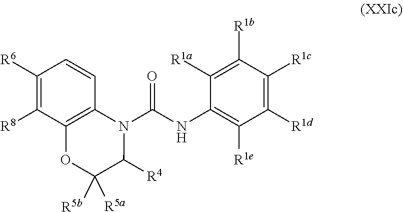

Including enantiomers, diasteromers, hydrates, solvates, pharmaceutically acceptable salts, prodrugs and complexes thereof.
The compounds of the present invention include compounds having formula (XXId):

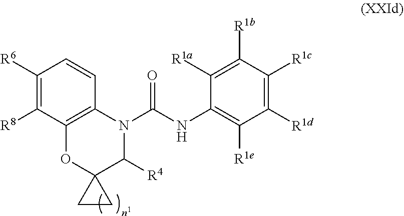

Including enantiomers, diasteromers, hydrates, solvates, pharmaceutically acceptable salts, prodrugs and complexes thereof.
The compounds of the present invention include compounds having formula (XXIe):

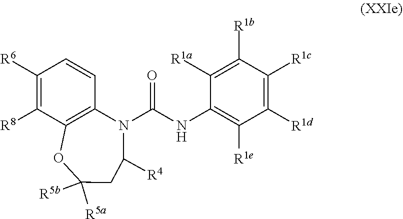

Including enantiomers, diasteromers, hydrates, solvates, pharmaceutically acceptable salts, prodrugs and complexes thereof.
The compounds of the present invention include compounds having formula (XXIf):

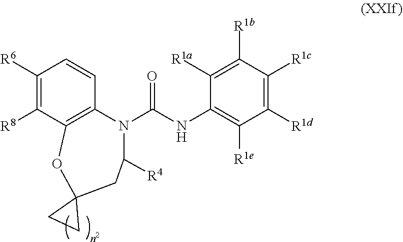

Including enantiomers, diasteromers, hydrates, solvates, pharmaceutically acceptable salts, prodrugs and complexes thereof
The compounds of the present invention include compounds having formula (XXII):

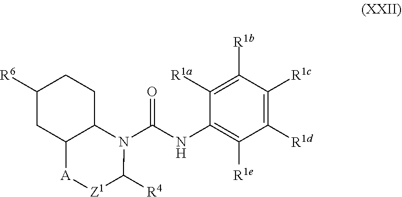

Including enantiomers, diasteromers, hydrates, solvates, pharmaceutically acceptable salts, prodrugs and complexes thereof,
The compounds of the present invention include compounds having formula (XXIII):

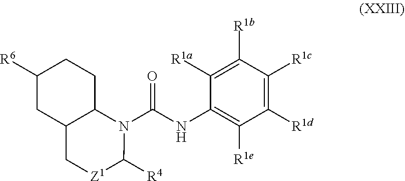

Including enantiomers, diasteromers, hydrates, solvates, pharmaceutically acceptable salts, prodrugs and complexes thereof.
The compounds of the present invention include compounds having formula (XXIIIa):

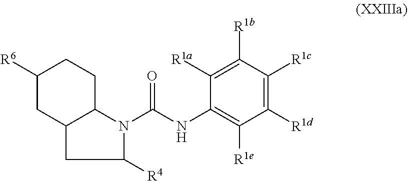

Including enantiomers, diasteromers, hydrates, solvates, pharmaceutically acceptable salts, prodrugs and complexes thereof.
The compounds of the present invention include compounds having formula (XXIIIb):

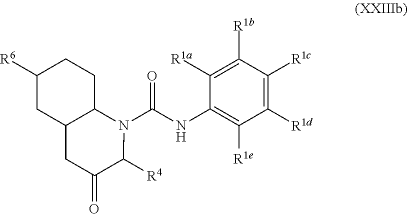

Including enantiomers, diasteromers, hydrates, solvates, pharmaceutically acceptable salts, prodrugs and complexes thereof.
The compounds of the present invention include compounds having formula (XXIIIc):

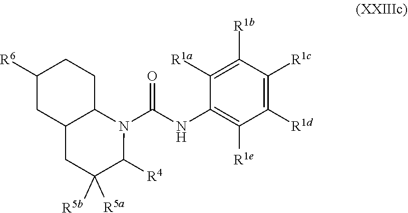

Including enantiomers, diasteromers, hydrates, solvates, pharmaceutically acceptable salts, prodrugs and complexes thereof.
The compounds of the present invention include compounds having formula (XXIIId):

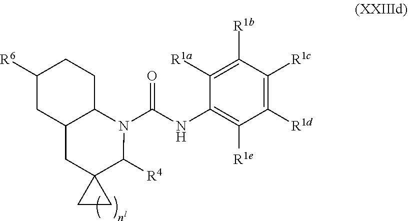

Including enantiomers, diasteromers, hydrates, solvates, pharmaceutically acceptable salts, prodrugs and complexes thereof.
The compounds of the present invention include compounds having formula (XXIIIe):

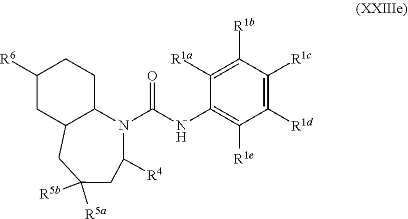

Including enantiomers, diasteromers, hydrates, solvates, pharmaceutically acceptable salts, prodrugs and complexes thereof.
The compounds of the present invention include compounds having formula (XXIIIf):

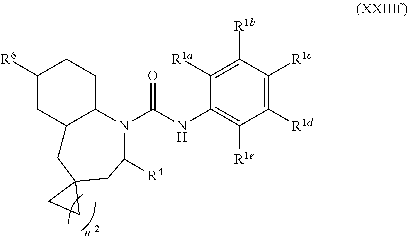

Including enantiomers, diasteromers, hydrates, solvates, pharmaceutically acceptable salts, prodrugs and complexes thereof.
The compounds of the present invention include compounds having formula (XXIV):

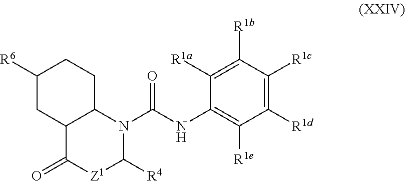

Including enantiomers, diasteromers, hydrates, solvates, pharmaceutically acceptable salts, prodrugs and complexes thereof.
The compounds of the present invention include compounds having formula (XXIVa):

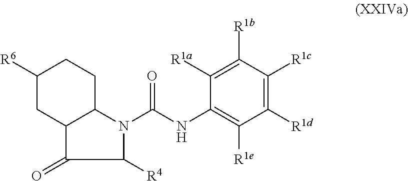

Including enantiomers, diasteromers, hydrates, solvates, pharmaceutically acceptable salts, prodrugs and complexes thereof.
The compounds of the present invention include compounds having formula (XXIVb):

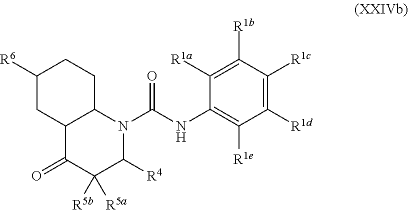

Including enantiomers, diasteromers, hydrates, solvates, pharmaceutically acceptable salts, prodrugs and complexes thereof.
The compounds of the present invention include compounds having formula (XXIVc):

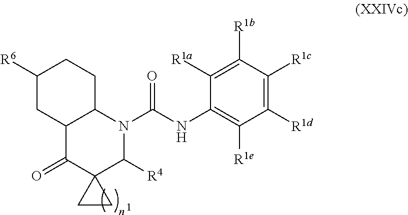

Including enantiomers, diasteromers, hydrates, solvates, pharmaceutically acceptable salts, prodrugs and complexes thereof.
The compounds of the present invention include compounds having formula (XXIVd):

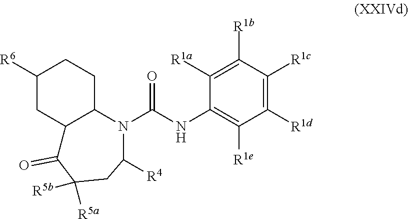

Including enantiomers, diasteromers, hydrates, solvates, pharmaceutically acceptable salts, prodrugs and complexes thereof.
The compounds of the present invention include compounds having formula (XXIVe):

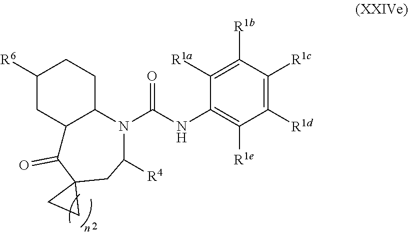

Including enantiomers, diasteromers, hydrates, solvates, pharmaceutically acceptable salts, prodrugs and complexes thereof.
The compounds of the present invention include compounds having formula (XXV):

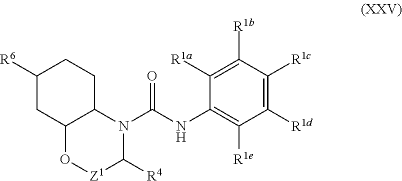

Including enantiomers, diasteromers, hydrates, solvates, pharmaceutically acceptable salts, prodrugs and complexes thereof.
The compounds of the present invention include compounds having formula (XXVa):

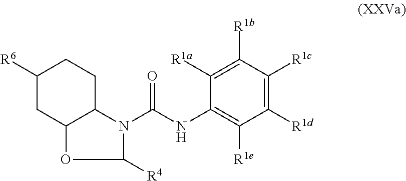

Including enantiomers, diasteromers, hydrates, solvates, pharmaceutically acceptable salts, prodrugs and complexes thereof.
The compounds of the present invention include compounds having formula (XXVb):

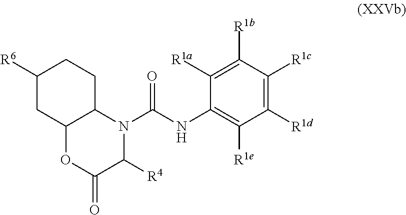

Including enantiomers, diasteromers, hydrates, solvates, pharmaceutically acceptable salts, prodrugs and complexes thereof.
The compounds of the present invention include compounds having formula (XXVc):

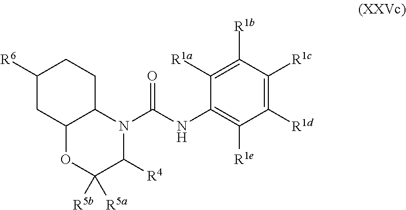

Including enantiomers, diasteromers, hydrates, solvates, pharmaceutically acceptable salts, prodrugs and complexes thereof.
The compounds of the present invention include compounds having formula (XXVd):

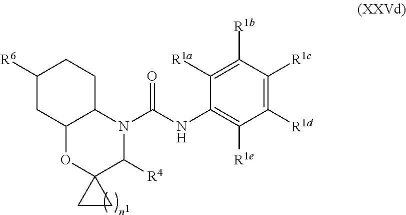

Including enantiomers, diasteromers, hydrates, solvates, pharmaceutically acceptable salts, prodrugs and complexes thereof.
The compounds of the present invention include compounds having formula (XXVe):

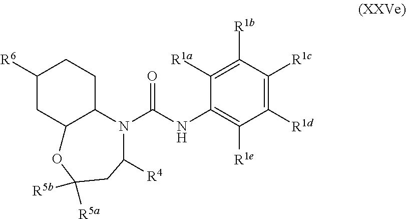

Including enantiomers, diasteromers, hydrates, solvates, pharmaceutically acceptable salts, prodrugs and complexes thereof.
The compounds of the present invention include compounds having formula (XXVf):

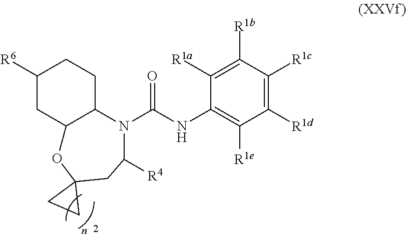

Including enantiomers, diasteromers, hydrates, solvates, pharmaceutically acceptable salts, prodrugs and complexes thereof.
The compounds of the present invention include compounds having formula (XXVI):

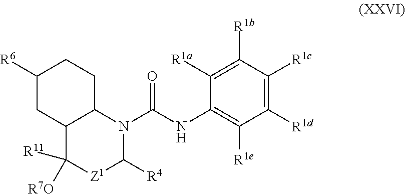

Including enantiomers, diasteromers, hydrates, solvates, pharmaceutically acceptable salts, prodrugs and complexes thereof.
The compounds of the present invention include compounds having formula (XXVIa):

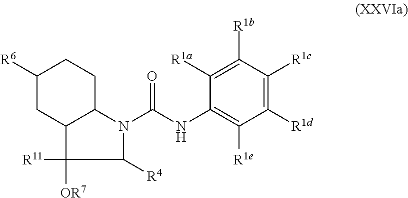

Including enantiomers, diasteromers, hydrates, solvates, pharmaceutically acceptable salts, prodrugs and complexes thereof.
The compounds of the present invention include compounds having formula (XXVIb):

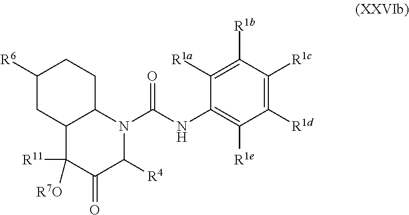

Including enantiomers, diasteromers, hydrates, solvates, pharmaceutically acceptable salts, prodrugs and complexes thereof.
The compounds of the present invention include compounds having formula (XXVIc):

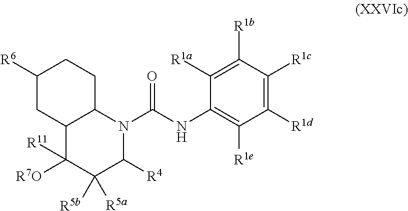

Including enantiomers, diasteromers, hydrates, solvates, pharmaceutically acceptable salts, prodrugs and complexes thereof.
The compounds of the present invention include compounds having formula (XXVId):

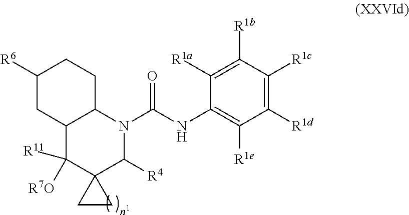

Including enantiomers, diasteromers, hydrates, solvates, pharmaceutically acceptable salts, prodrugs and complexes thereof.
The compounds of the present invention include compounds having formula (XXVIe):

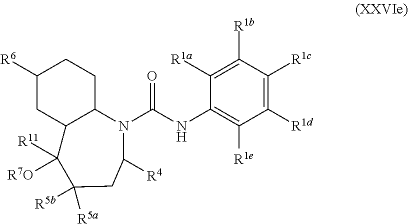

Including enantiomers, diasteromers, hydrates, solvates, pharmaceutically acceptable salts, prodrugs and complexes thereof.
The compounds of the present invention include compounds having formula (XXVIf):

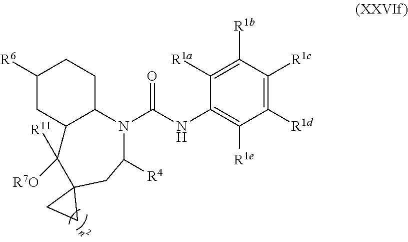

Including enantiomers, diasteromers, hydrates, solvates, pharmaceutically acceptable salts, prodrugs and complexes thereof.
The compounds of the present invention include compounds having formula (XXVII):

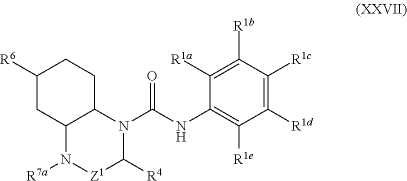

Including enantiomers, diasteromers, hydrates, solvates, pharmaceutically acceptable salts, prodrugs and complexes thereof.
The compounds of the present invention include compounds having formula (XXVIIa):

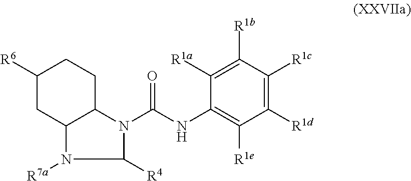

Including enantiomers, diasteromers, hydrates, solvates, pharmaceutically acceptable salts, prodrugs and complexes thereof.
The compounds of the present invention include compounds having formula (XXVIIb):

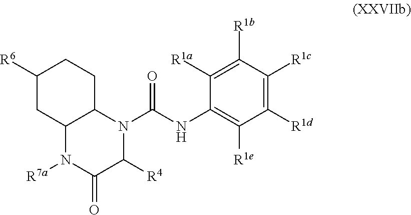

Including enantiomers, diasteromers, hydrates, solvates, pharmaceutically acceptable salts, prodrugs and complexes thereof.
The compounds of the present invention include compounds having formula (XXVIIc):

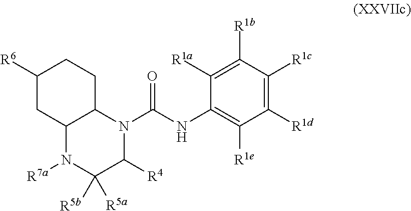

Including enantiomers, diasteromers, hydrates, solvates, pharmaceutically acceptable salts, prodrugs and complexes thereof.
The compounds of the present invention include compounds having formula (XXVIId):

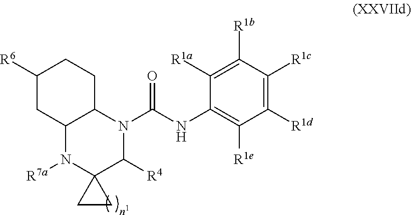

Including enantiomers, diasteromers, hydrates, solvates, pharmaceutically acceptable salts, prodrugs and complexes thereof.
The compounds of the present invention include compounds having formula (XXVIIe):

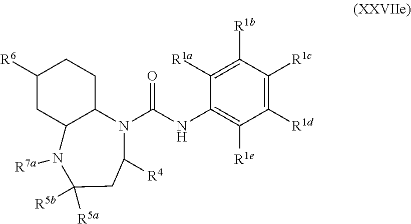

Including enantiomers, diasteromers, hydrates, solvates, pharmaceutically acceptable salts, prodrugs and complexes thereof.
The compounds of the present invention include compounds having formula (XXVIIf):

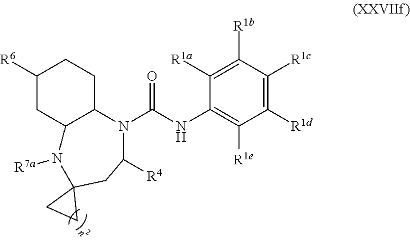

Including enantiomers, diasteromers, hydrates, solvates, pharmaceutically acceptable salts, prodrugs and complexes thereof.
The compounds of the present invention include compounds having formula (XXVIII):

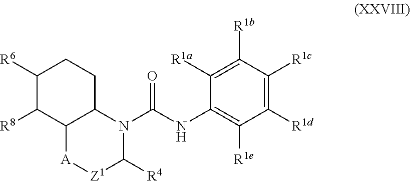

Including enantiomers, diasteromers, hydrates, solvates, pharmaceutically acceptable salts, prodrugs and complexes thereof.
The compounds of the present invention include compounds having formula (XXVIIIa):

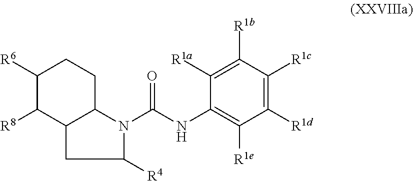

Including enantiomers, diasteromers, hydrates, solvates, pharmaceutically acceptable salts, prodrugs and complexes thereof.
The compounds of the present invention include compounds having formula (XXVIIIb):

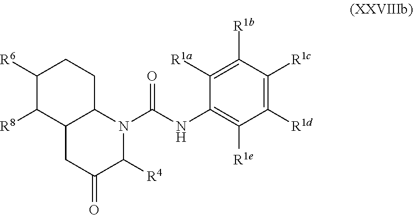

Including enantiomers, diasteromers, hydrates, solvates, pharmaceutically acceptable salts, prodrugs and complexes thereof.
The compounds of the present invention include compounds having formula (XXVIIIc):

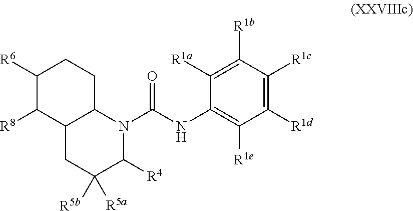

Including enantiomers, diasteromers, hydrates, solvates, pharmaceutically acceptable salts, prodrugs and complexes thereof.
The compounds of the present invention include compounds having formula (XXVIIId):

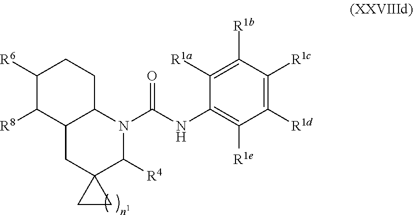

Including enantiomers, diasteromers, hydrates, solvates, pharmaceutically acceptable salts, prodrugs and complexes thereof.
The compounds of the present invention include compounds having formula (XXVIIIe):

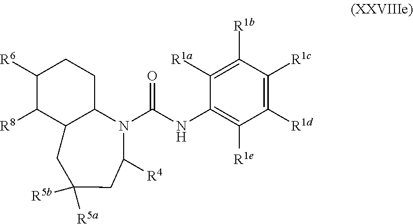

Including enantiomers, diasteromers, hydrates, solvates, pharmaceutically acceptable salts, prodrugs and complexes thereof.
The compounds of the present invention include compounds having formula (XXVIIIf):

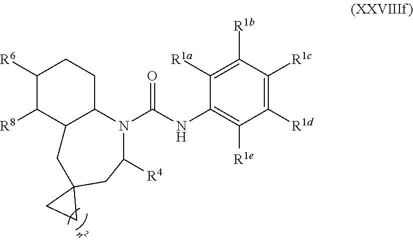

Including enantiomers, diasteromers, hydrates, solvates, pharmaceutically acceptable salts, prodrugs and complexes thereof.
The compounds of the present invention include compounds having formula (XXIX):

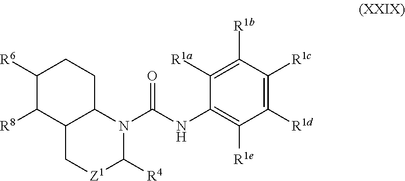

Including enantiomers, diasteromers, hydrates, solvates, pharmaceutically acceptable salts, prodrugs and complexes thereof.
The compounds of the present invention include compounds having formula (XXIXa):

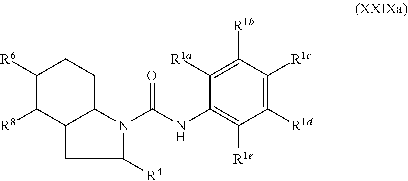

Including enantiomers, diasteromers, hydrates, solvates, pharmaceutically acceptable salts, prodrugs and complexes thereof.
The compounds of the present invention include compounds having formula (XXIXb):

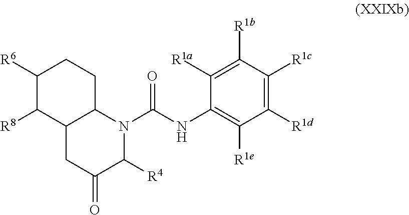

Including enantiomers, diasteromers, hydrates, solvates, pharmaceutically acceptable salts, prodrugs and complexes thereof.
The compounds of the present invention include compounds having formula (XXIXc):

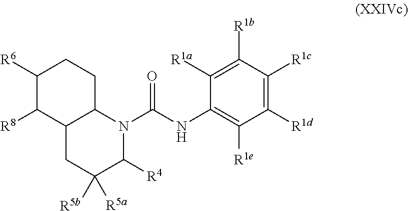

Including enantiomers, diasteromers, hydrates, solvates, pharmaceutically acceptable salts, prodrugs and complexes thereof.
The compounds of the present invention include compounds having formula (XXIXd):

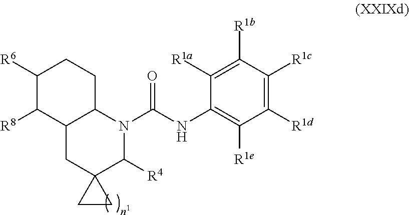

Including enantiomers, diasteromers, hydrates, solvates, pharmaceutically acceptable salts, prodrugs and complexes thereof.
The compounds of the present invention include compounds having formula (XXIXe):

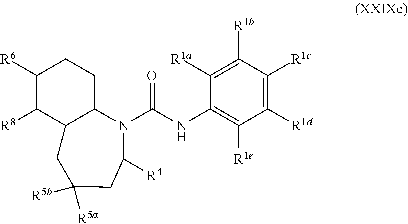

Including enantiomers, diasteromers, hydrates, solvates, pharmaceutically acceptable salts, prodrugs and complexes thereof.
The compounds of the present invention include compounds having formula (XXIXf):

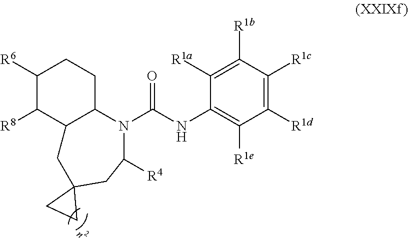

Including enantiomers, diasteromers, hydrates, solvates, pharmaceutically acceptable salts, prodrugs and complexes thereof.
The compounds of the present invention include compounds having formula (XXX):

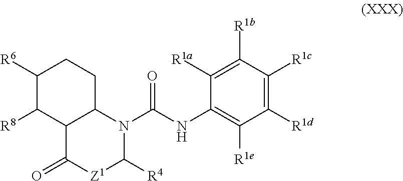

Including enantiomers, diasteromers, hydrates, solvates, pharmaceutically acceptable salts, prodrugs and complexes thereof.
The compounds of the present invention include compounds having formula (XXXa):

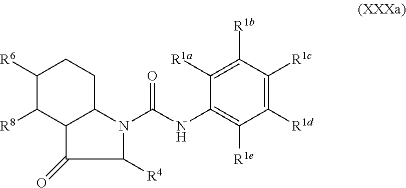

Including enantiomers, diasteromers, hydrates, solvates, pharmaceutically acceptable salts, prodrugs and complexes thereof.
The compounds of the present invention include compounds having formula (XXXb):

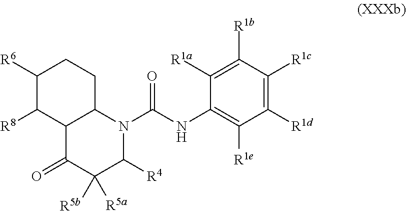

Including enantiomers, diasteromers, hydrates, solvates, pharmaceutically acceptable salts, prodrugs and complexes thereof.
The compounds of the present invention include compounds having formula (XXXc):

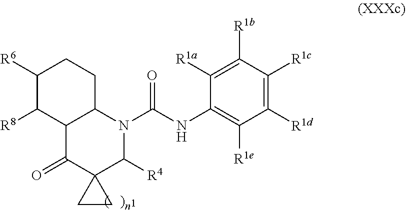

Including enantiomers, diasteromers, hydrates, solvates, pharmaceutically acceptable salts, prodrugs and complexes thereof.
The compounds of the present invention include compounds having formula (XXXd):

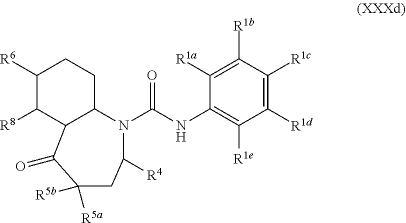

Including enantiomers, diasteromers, hydrates, solvates, pharmaceutically acceptable salts, prodrugs and complexes thereof.
The compounds of the present invention include compounds having formula (XXXe):

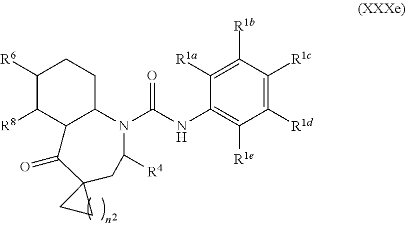

Including enantiomers, diasteromers, hydrates, solvates, pharmaceutically acceptable salts, prodrugs and complexes thereof.
The compounds of the present invention include compounds having formula (XXXI):

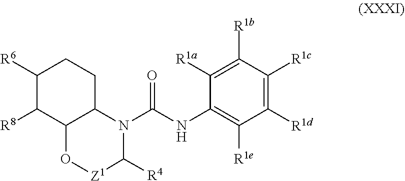

Including enantiomers, diasteromers, hydrates, solvates, pharmaceutically acceptable salts, prodrugs and complexes thereof.
The compounds of the present invention include compounds having formula (XXXIa):

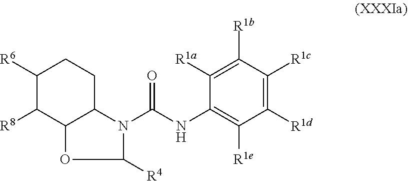

Including enantiomers, diasteromers, hydrates, solvates, pharmaceutically acceptable salts, prodrugs and complexes thereof.
The compounds of the present invention include compounds having formula (XXXIb):

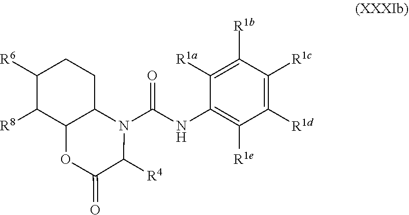

Including enantiomers, diasteromers, hydrates, solvates, pharmaceutically acceptable salts, prodrugs and complexes thereof.
The compounds of the present invention include compounds having formula (XXXIc):

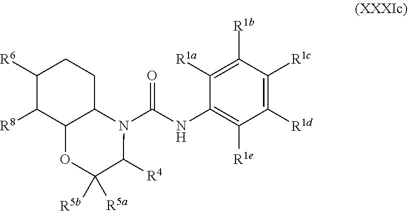

Including enantiomers, diasteromers, hydrates, solvates, pharmaceutically acceptable salts, prodrugs and complexes thereof.
The compounds of the present invention include compounds having formula (XXXId):

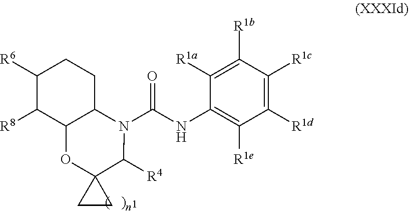

Including enantiomers, diasteromers, hydrates, solvates, pharmaceutically acceptable salts, prodrugs and complexes thereof.
The compounds of the present invention include compounds having formula (XXXIe):

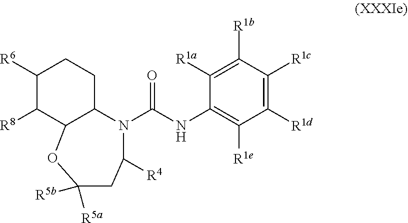

Including enantiomers, diasteromers, hydrates, solvates, pharmaceutically acceptable salts, prodrugs and complexes thereof.
The compounds of the present invention include compounds having formula (XXXIf):

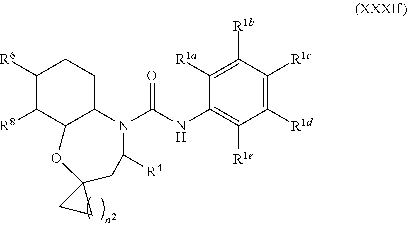

Including enantiomers, diasteromers, hydrates, solvates, pharmaceutically acceptable salts, prodrugs and complexes thereof.
The compounds of the present invention include compounds having formula (XXXII):

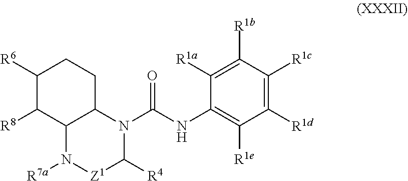

Including enantiomers, diasteromers, hydrates, solvates, pharmaceutically acceptable salts, prodrugs and complexes thereof.
The compounds of the present invention include compounds having formula (XXXIIa):

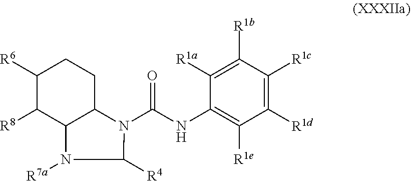

Including enantiomers, diasteromers, hydrates, solvates, pharmaceutically acceptable salts, prodrugs and complexes thereof.
The compounds of the present invention include compounds having formula (XXXIIb):

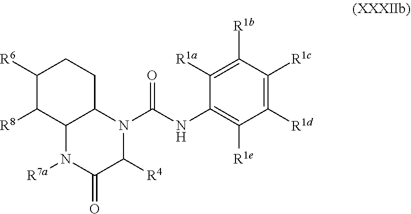

Including enantiomers, diasteromers, hydrates, solvates, pharmaceutically acceptable salts, prodrugs and complexes thereof.
The compounds of the present invention include compounds having formula (XXXIIc):

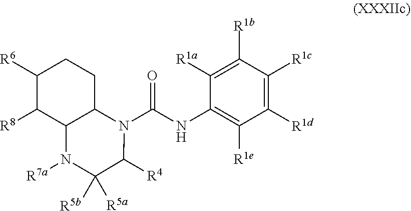

Including enantiomers, diasteromers, hydrates, solvates, pharmaceutically acceptable salts, prodrugs and complexes thereof.
The compounds of the present invention include compounds having formula (XXXIId):

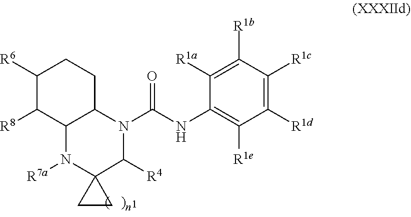

Including enantiomers, diasteromers, hydrates, solvates, pharmaceutically acceptable salts, prodrugs and complexes thereof.
The compounds of the present invention include compounds having formula (XXXIIe):

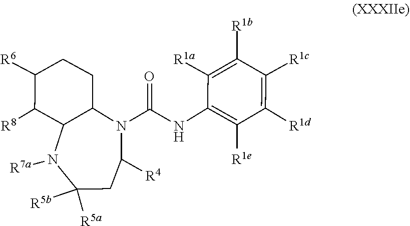

Including enantiomers, diasteromers, hydrates, solvates, pharmaceutically acceptable salts, prodrugs and complexes thereof.
The compounds of the present invention include compounds having formula (XXXIIf):

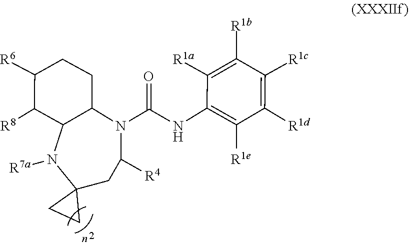

Including enantiomers, diasteromers, hydrates, solvates, pharmaceutically acceptable salts, prodrugs and complexes thereof.
The compounds of the present invention include compounds having formula (XXXIII):

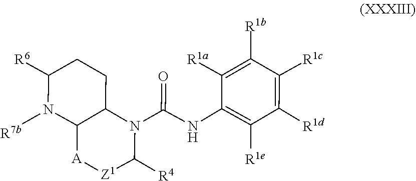

Including enantiomers, diasteromers, hydrates, solvates, pharmaceutically acceptable salts, prodrugs and complexes thereof.
The compounds of the present invention include compounds having formula (XXXIV):

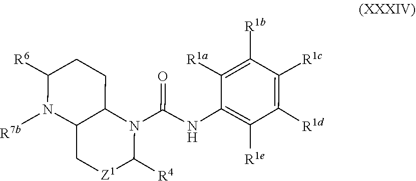

Including enantiomers, diasteromers, hydrates, solvates, pharmaceutically acceptable salts, prodrugs and complexes thereof.
The compounds of the present invention include compounds having formula (XXXIVa):

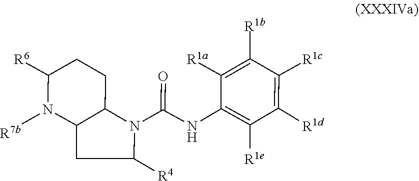

Including enantiomers, diasteromers, hydrates, solvates, pharmaceutically acceptable salts, prodrugs and complexes thereof.
The compounds of the present invention include compounds having formula (XXXIVb):

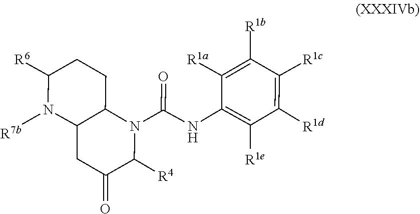

Including enantiomers, diasteromers, hydrates, solvates, pharmaceutically acceptable salts, prodrugs and complexes thereof.
The compounds of the present invention include compounds having formula (XXXIVc):

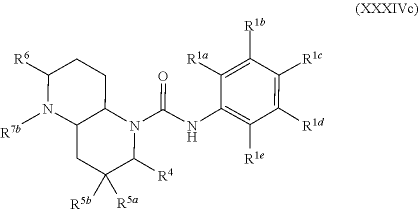

Including enantiomers, diasteromers, hydrates, solvates, pharmaceutically acceptable salts, prodrugs and complexes thereof.
The compounds of the present invention include compounds having formula (XXXIVd):

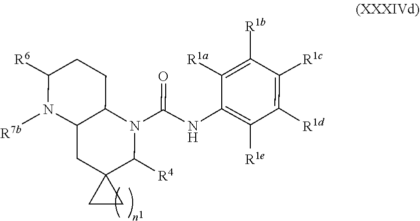

Including enantiomers, diasteromers, hydrates, solvates, pharmaceutically acceptable salts, prodrugs and complexes thereof.
The compounds of the present invention include compounds having formula (XXXIVe):

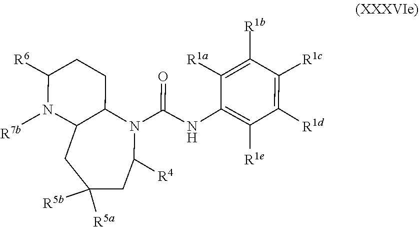

Including enantiomers, diasteromers, hydrates, solvates, pharmaceutically acceptable salts, prodrugs and complexes thereof.
The compounds of the present invention include compounds having formula (XXXIVf):

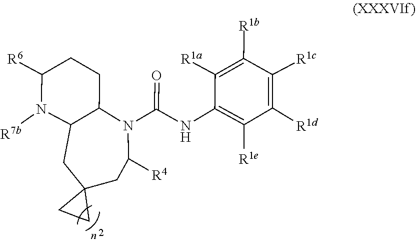

Including enantiomers, diasteromers, hydrates, solvates, pharmaceutically acceptable salts, prodrugs and complexes thereof.
The compounds of the present invention include compounds having formula (XXXV):

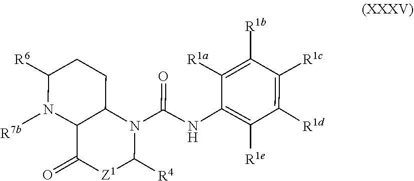

Including enantiomers, diasteromers, hydrates, solvates, pharmaceutically acceptable salts, prodrugs and complexes thereof.
The compounds of the present invention include compounds having formula (XXXVa):

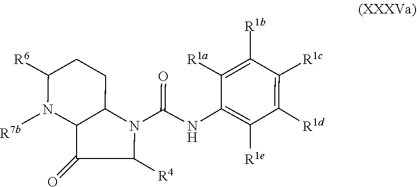

Including enantiomers, diasteromers, hydrates, solvates, pharmaceutically acceptable salts, prodrugs and complexes thereof.
The compounds of the present invention include compounds having formula (XXXVb):

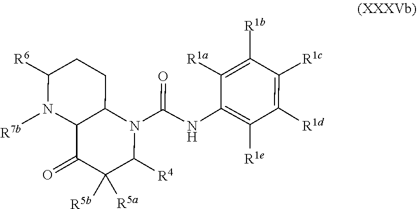

Including enantiomers, diasteromers, hydrates, solvates, pharmaceutically acceptable salts, prodrugs and complexes thereof.
The compounds of the present invention include compounds having formula (XXXVc):

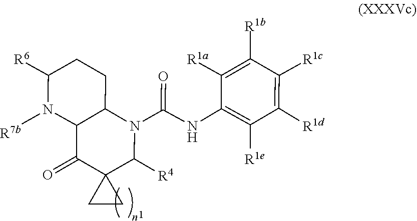

Including enantiomers, diasteromers, hydrates, solvates, pharmaceutically acceptable salts, prodrugs and complexes thereof.
The compounds of the present invention include compounds having formula (XXXVd):

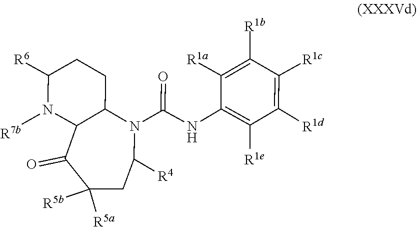

Including enantiomers, diasteromers, hydrates, solvates, pharmaceutically acceptable salts, prodrugs and complexes thereof.
The compounds of the present invention include compounds having formula (XXXVe):

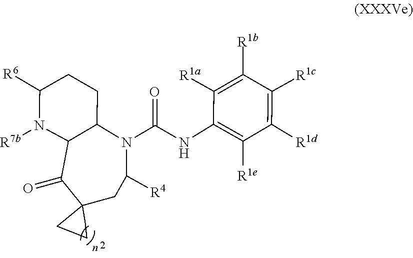

Including enantiomers, diasteromers, hydrates, solvates, pharmaceutically acceptable salts, prodrugs and complexes thereof.
The compounds of the present invention include compounds having formula (XXXVI):

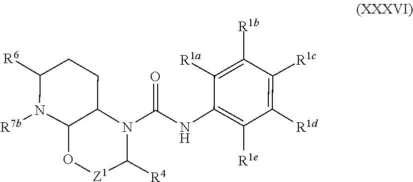

Including enantiomers, diasteromers, hydrates, solvates, pharmaceutically acceptable salts, prodrugs and complexes thereof.
The compounds of the present invention include compounds having formula (XXXVIa):

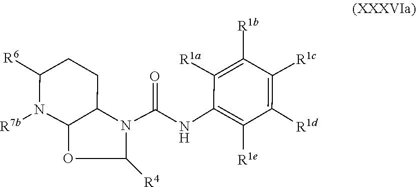

Including enantiomers, diasteromers, hydrates, solvates, pharmaceutically acceptable salts, prodrugs and complexes thereof.
The compounds of the present invention include compounds having formula (XXXVIb):

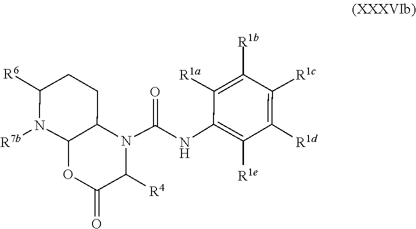

Including enantiomers, diasteromers, hydrates, solvates, pharmaceutically acceptable salts, prodrugs and complexes thereof.
The compounds of the present invention include compounds having formula (XXXVIc):

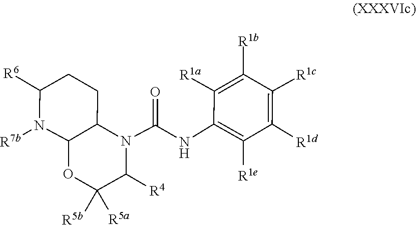

Including enantiomers, diasteromers, hydrates, solvates, pharmaceutically acceptable salts, prodrugs and complexes thereof.
The compounds of the present invention include compounds having formula (XXXVId):

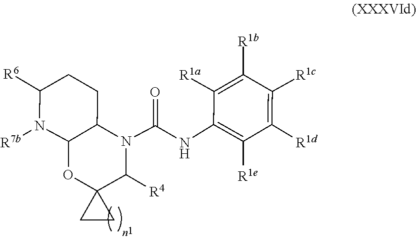

Including enantiomers, diasteromers, hydrates, solvates, pharmaceutically acceptable salts, prodrugs and complexes thereof.
The compounds of the present invention include compounds having formula (XXXVIe):

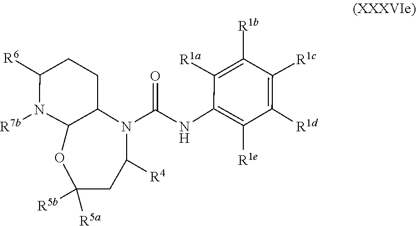

Including enantiomers, diasteromers, hydrates, solvates, pharmaceutically acceptable salts, prodrugs and complexes thereof.
The compounds of the present invention include compounds having formula (XXXVIf):

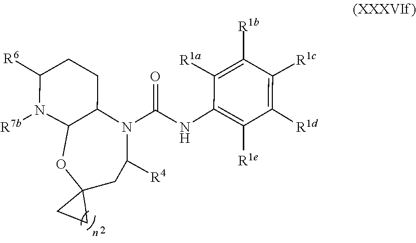

Including enantiomers, diasteromers, hydrates, solvates, pharmaceutically acceptable salts, prodrugs and complexes thereof.
The compounds of the present invention include compounds having formula (XXXVII):

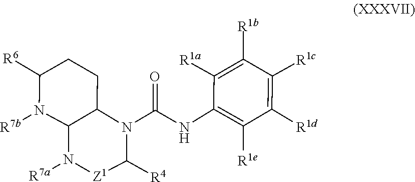

Including enantiomers, diasteromers, hydrates, solvates, pharmaceutically acceptable salts, prodrugs and complexes thereof.
The compounds of the present invention include compounds having formula (XXXVIIa):

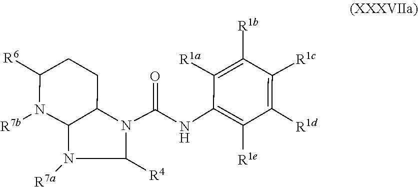

Including enantiomers, diasteromers, hydrates, solvates, pharmaceutically acceptable salts, prodrugs and complexes thereof.
The compounds of the present invention include compounds having formula (XXXVIIb):

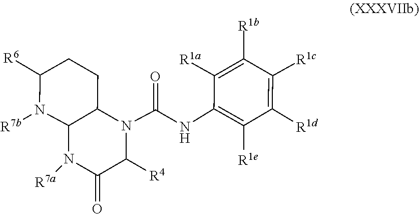

Including enantiomers, diasteromers, hydrates, solvates, pharmaceutically acceptable salts, prodrugs and complexes thereof.
The compounds of the present invention include compounds having formula (XXXVIIc):

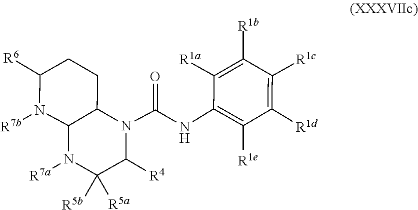

Including enantiomers, diasteromers, hydrates, solvates, pharmaceutically acceptable salts, prodrugs and complexes thereof.
The compounds of the present invention include compounds having formula (XXXVIId):

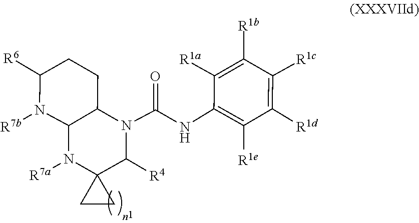

Including enantiomers, diasteromers, hydrates, solvates, pharmaceutically acceptable salts, prodrugs and complexes thereof.
The compounds of the present invention include compounds having formula (XXXVIIe):

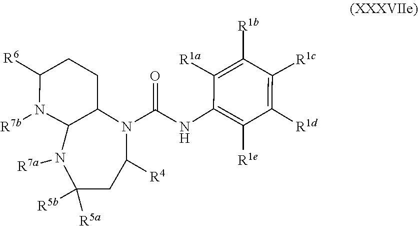

Including enantiomers, diasteromers, hydrates, solvates, pharmaceutically acceptable salts, prodrugs and complexes thereof.
The compounds of the present invention include compounds having formula (XXXVIIf):

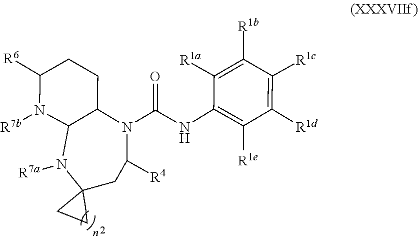

Including enantiomers, diasteromers, hydrates, solvates, pharmaceutically acceptable salts, prodrugs and complexes thereof.

In some embodiments A is CH₂.

In some embodiments A is C═O.

In some embodiments A is oxygen.

In some embodiments A is

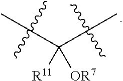

In some embodiments A is NR^{7a}.

In some embodiments Z¹ is bond,

In some embodiments Z¹ is C═O).

In some embodiments Z¹ is

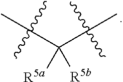

In some embodiments Z¹ is

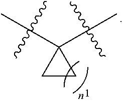

In some embodiments Z¹ is

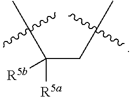

In some embodiments Z¹ is

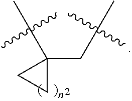

In some embodiments n¹ is 1.

In some embodiments n¹ is 2.

In some embodiments n¹ is 3.

In some embodiments n¹ is 4.

In some embodiments n¹ is 5.

In some embodiments n² is 1.

In some embodiments n² is 2.

In some embodiments n² is 3.

In some embodiments n² is 4.

In some embodiments n² is 5.

In some embodiments Q¹ is an aromatic ring containing 5 ring atoms.

In some embodiments Q¹ is an aromatic ring containing 6 ring atoms.

In some embodiments Q¹ is a saturated ring containing 5 ring atoms.

In some embodiments Q¹ is a saturated ring containing 6 ring atoms.

In some embodiments Q¹ is a saturated ring containing 7 ring atoms.

In some embodiments X is CR⁸.

In some embodiments X is nitrogen.

In some embodiments X is CH₂.

In some embodiments X is CHR⁸.

In some embodiments X is NR^{7b}.

In some embodiments R^{1a} is hydrogen.

In some embodiments R^{1a} is fluorine.

In some embodiments R^{1a} is chlorine.

In some embodiments R^{1a} is bromine.

In some embodiments R^{1a} is —CHF₂.

In some embodiments R^{1a} is —CH₂F.

In some embodiments R^{1a} is —CF₃.

In some embodiments R^{1a} is —CN.

In some embodiments R^{1a} is OR⁹.

In some embodiments R^{1a} is C₁₋₆ alkyl.

In some embodiments R^{1a} is C₃₋₅ cycloalkyl.

In some embodiments R^{1b} is hydrogen.

In some embodiments R^{1b} is fluorine.

In some embodiments R^{1b} is chlorine.

In some embodiments R^{1b} is bromine.

In some embodiments R^{1b} is —CHF₂.

In some embodiments R^{1b} is —CH₂F.

In some embodiments R^{1b} is —CF₃.

In some embodiments R^{1b} is —CN.

In some embodiments R^{1b} is OR⁹.

In some embodiments R^{1b} is C₁₋₆ alkyl.

In some embodiments R^{1b} is C₃₋₅ cycloalkyl.

In some embodiments R^{1c} is hydrogen.

In some embodiments R^{1c} is fluorine.

In some embodiments R^{1c} is chlorine.

In some embodiments R^{1c} is bromine.

In some embodiments R^{1c} is —CHF₂.

In some embodiments R^{1c} is —CH₂F.

In some embodiments R^{1c} is —CF₃.

In some embodiments R^{1c} is —CN.

In some embodiments R^{1c} is OR⁹.

In some embodiments R^{1c} is C₁₋₆ alkyl.

In some embodiments R^{1c} is C₃₋₅ cycloalkyl.

In some embodiments R^{1d} is hydrogen.

In some embodiments R^{1d} is fluorine.

In some embodiments R^{1d} is chlorine.

In some embodiments R^{1d} is bromine.

In some embodiments R^{1d} is —CHF₂.

In some embodiments R^{1d} is —CH₂F.

In some embodiments R^{1d} is —CF₃.

In some embodiments R^{1d} is —CN.

In some embodiments R^{1d} is OR⁹.

In some embodiments R^{1d} is C₁₋₆ alkyl.

In some embodiments R^{1d} is C₃₋₅ cycloalkyl.

In some embodiments R^{1e} is hydrogen.

In some embodiments R^{1e} is fluorine.

In some embodiments R^{1e} is chlorine.

In some embodiments R^{1e} is bromine.

In some embodiments R^{1e} is —CHF₂.

In some embodiments R^{1e} is —CH₂F.

In some embodiments R^{1e} is —CF₃.

In some embodiments R^{1e} is —CN.

In some embodiments R^{1e} is OR⁹.

In some embodiments R^{1e} is C₁₋₆ alkyl.

In some embodiments R^{1e} is C₃₋₅ cycloalkyl.

In some embodiments R^{1b} and R^{1c} are taken together with the atom to which they are bound to form ring having 5 ring atoms optionally containing 1 groups selected from oxygen and NH.

In some embodiments R^{1b} and R^{1c} are taken together with the atom to which they are bound to form an optionally substituted ring having 5 ring atoms optionally containing 1 groups selected from oxygen and NH.

In some embodiments R^{1b} and R^{1c} are taken together with the atom to which they are bound to form ring having 5 ring atoms optionally containing 2 groups selected from oxygen and NH.

In some embodiments R^{1b} and R^{1c} are taken together with the atom to which they are bound to form an optionally substituted ring having 5 ring atoms optionally containing 2 groups selected from oxygen and NH.

In some embodiments R^{1b} and R^{1c} are taken together with the atom to which they are bound to form ring having 6 ring atoms optionally containing 1 groups selected from oxygen and NH.

In some embodiments R^{1b} and R^{1c} are taken together with the atom to which they are bound to form an optionally substituted ring having 6 ring atoms optionally containing 1 groups selected from oxygen and NH.

In some embodiments R^{1b} and R^{1c} are taken together with the atom to which they are bound to form ring having 6 ring atoms optionally containing 2 groups selected from oxygen and NH.

In some embodiments R^{1b} and R^{1c} are taken together with the atom to which they are bound to form an optionally substituted ring having 6 ring atoms optionally containing 2 groups selected from oxygen and NH.

In some embodiments R^{1b} and R^{1c} are taken together with the atom to which they are bound to form ring having 7 ring atoms optionally containing 1 groups selected from oxygen and NH.

In some embodiments R^{1b} and R^{1c} are taken together with the atom to which they are bound to form an optionally substituted ring having 7 ring atoms optionally containing 1 groups selected from oxygen and NH.

In some embodiments R^{1b} and R^{1c} are taken together with the atom to which they are bound to form ring having 7 ring atoms optionally containing 2 groups selected from oxygen and NH.

In some embodiments R^{1b} and R^{1c} are taken together with the atom to which they are bound to form an optionally substituted ring having 7 ring atoms optionally containing 2 groups selected from oxygen and NH.

In some embodiments R^{1c} and R^{1d} are taken together with the atom to which they are bound to form ring having 5 ring atoms optionally containing 1 groups selected from oxygen and NH.

In some embodiments R^{1c} and R^{1d} are taken together with the atom to which they are bound to form an optionally substituted ring having 5 ring atoms optionally containing 1 groups selected from oxygen and NH.

In some embodiments R^{1c} and R^{1d} are taken together with the atom to which they are bound to form ring having 5 ring atoms optionally containing 2 groups selected from oxygen and NH.

In some embodiments R^{1c} and R^{1d} are taken together with the atom to which they are bound to form an optionally substituted ring having 5 ring atoms optionally containing 2 groups selected from oxygen and NH.

In some embodiments R^{1c} and R^{1d} are taken together with the atom to which they are bound to form ring having 6 ring atoms optionally containing 1 groups selected from oxygen and NH.

In some embodiments R^{1c} and R^{1d} are taken together with the atom to which they are bound to form an optionally substituted ring having 6 ring atoms optionally containing 1 groups selected from oxygen and NH.

In some embodiments R^{1c} and R^{1d} are taken together with the atom to which they are bound to form ring having 6 ring atoms optionally containing 2 groups selected from oxygen and NH.

In some embodiments R^{1c} and R^{1d} are taken together with the atom to which they are bound to form an optionally substituted ring having 6 ring atoms optionally containing 2 groups selected from oxygen and NH.

In some embodiments R^{1c} and R^{1d} are taken together with the atom to which they are bound to form ring having 7 ring atoms optionally containing 1 groups selected from oxygen and NH.

In some embodiments R^{1c} and R^{1d} are taken together with the atom to which they are bound to form an optionally substituted ring having 7 ring atoms optionally containing 1 groups selected from oxygen and NH.

In some embodiments R^{1c} and R^{1d} are taken together with the atom to which they are bound to form ring having 7 ring atoms optionally containing 2 groups selected from oxygen and NH.

In some embodiments R^{1c} and R^{1d} are taken together with the atom to which they are bound to form an optionally substituted ring having 7 ring atoms optionally containing 2 groups selected from oxygen and NH.

In some embodiments R⁴ is hydrogen.

In some embodiments R⁴ is C₁₋₆ linear alkyl.

In some embodiments R⁴ is C₃₋₇ branched alkyl.

In some embodiments R⁴ is

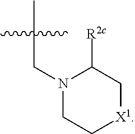

In some embodiments R⁴ is

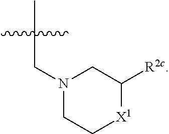

In some embodiments R⁴ is

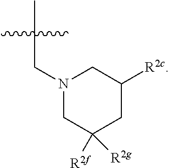

In some embodiments R⁴ is

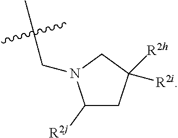

In some embodiments R⁴ is optionally substituted C₃₋₈ cycloalkyl.

In some embodiments R⁴ is optionally substituted aryl.

In some embodiments R⁴ is a phenyl ring.

In some embodiments R⁴ is a phenyl ring that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁴ is a phenyl ring that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁴ is a phenyl ring that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁴ is optionally substituted heteroaryl.

In some embodiments R⁴ is a heteroaryl that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁴ is a heteroaryl that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁴ is a heteroaryl that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments X¹ is oxygen.

In some embodiments X¹ is CR^{2a}R^{2b}.

In some embodiments X¹ is C═O.

In some embodiments X¹ is SO₂.

In some embodiments X¹ is NSO₂R^{2d}.

In some embodiments R^{2a} is hydrogen.

In some embodiments R^{2a} is fluorine.

In some embodiments R^{2a} is chlorine.

In some embodiments R^{2a} is C₁₋₆ linear alkyl.

In some embodiments R^{2a} is C₃₋₇ branched alkyl.

In some embodiments R^{2a} is C₁₋₆ linear alkoxy.

In some embodiments R^{2a} is C₃₋₇ branched alkoxy.

In some embodiments R^{2b} is hydrogen.

In some embodiments R^{2b} is fluorine.

In some embodiments R^{2b} is chlorine.

In some embodiments R^{2b} is C₁₋₆ linear alkyl.

In some embodiments R^{2b} is C₃₋₇ branched alkyl.

In some embodiments R^{2b} is C₁₋₆ linear alkoxy.

In some embodiments R^{2b} is C₃₋₇ branched alkoxy.

In some embodiments R^{2c} is hydrogen.

In some embodiments R^{2c} is CO₂R^{2e}.

In some embodiments R^{2c} is optionally substituted C₁₋₆ linear alkyl.

In some embodiments R^{2c} is C₁₋₆ linear alkyl that is optionally substituted with a group selected from OH, CO₂H, and CO₂R^{2e}.

In some embodiments R^{2c} is C₃₋₇ branched alkyl that is optionally substituted with a group selected from OH, CO₂H, and CO₂R^{2e}.

In some embodiments R^{2c} is optionally substitute C₃₋₇ branched alkyl.

In some embodiments R^{2a} is C₁₋₆ linear alkyl.

In some embodiments R^{2d} is C₃₋₇ branched alkyl.

In some embodiments R^{2e} is hydrogen.

In some embodiments R^{2e} is C₁₋₆ linear alkyl.

In some embodiments R^{2e} is C₃₋₇ branched alkyl.

In some embodiments R^{2f} is hydrogen.

In some embodiments R^{2f} is fluorine.

In some embodiments R^{2f} is chlorine.

In some embodiments R^{2f} is C₁₋₆ linear alkyl.

In some embodiments R^{2f} is C₃₋₇ branched alkyl.

In some embodiments R^{2f} is C₁₋₆ linear alkoxy.

In some embodiments R^{2f} is C₃₋₇ branched alkoxy.

In some embodiments R^{2g} is hydrogen.

In some embodiments R^{2g} is fluorine.

In some embodiments R^{2g} is chlorine.

In some embodiments R^{2g} is C₁₋₆ linear alkyl.

In some embodiments R^{2g} is C₃₋₇ branched alkyl.

In some embodiments R^{2g} is C₁₋₆ linear alkoxy.

In some embodiments R^{2g} is C₃₋₇ branched alkoxy.

In some embodiments R^{2h} is hydrogen.

In some embodiments R^{2h} is fluorine.

In some embodiments R^{2h} is chlorine.

In some embodiments R^{2h} is C₁₋₆ linear alkyl.

In some embodiments R^{2h} is C₃₋₇ branched alkyl.

In some embodiments R^{2h} is C₁₋₆ linear alkoxy.

In some embodiments R^{2h} is C₃₋₇ branched alkoxy.

In some embodiments R²ⁱ is hydrogen.

In some embodiments R²ⁱ is fluorine.

In some embodiments R²ⁱ is chlorine.

In some embodiments R²ⁱ is C₁₋₆ linear alkyl.

In some embodiments R²ⁱ is C₃₋₇ branched alkyl.

In some embodiments R²ⁱ is C₁₋₆ linear alkoxy.

In some embodiments R²ⁱ is C₃₋₇ branched alkoxy.

In some embodiments R^{2j} is hydrogen.

In some embodiments R^{2j} is CO₂R^{2e}.

In some embodiments R^{2j} is optionally substituted C₁₋₆ linear alkyl.

In some embodiments R^{2j} is optionally substitute C₃₋₇ branched alkyl.

In some embodiments R^{2j} is C₁₋₆ linear alkyl that is optionally substituted with a group selected from OH, CO₂H, and CO₂R^{2e}.

In some embodiments R^{2j} is C₃₋₇ branched alkyl that is optionally substituted with a group selected from OH, CO₂H, and CO₂R^{2e}.

In some embodiments R^{5a} is hydrogen.

In some embodiments R^{5a} is C₁₋₆ linear alkyl.

In some embodiments R^{5a} is C₃₋₇ branched alkyl.

In some embodiments R^{5a} is CONH₂.

In some embodiments R^{5a} is

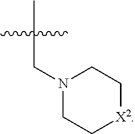

In some embodiments R^{5a} is

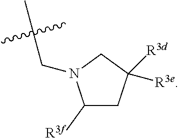

In some embodiments R^{5a} is optionally substituted aryl.

In some embodiments R^{5a} is a phenyl ring.

In some embodiments R^{5a} is a phenyl ring that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R^{5a} is a phenyl ring that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R^{5a} is a phenyl ring that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R^{5a} is optionally substituted heteroaryl.

In some embodiments R^{5a} is a heteroaryl that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R^{5a} is a heteroaryl that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R^{5a} is a heteroaryl that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R^{5b} is hydrogen.

In some embodiments R^{5b} is C₁₋₆ linear alkyl.

In some embodiments R^{5b} is C₃₋₇ branched alkyl.

In some embodiments X² is oxygen.

In some embodiments X² is CR^{3a}R^{3b}.

In some embodiments X² is C═O.

In some embodiments X² is SO₂.

In some embodiments X² is NSO₂R^{3c}.

In some embodiments R^{3a} is hydrogen.

In some embodiments R^{3a} is fluorine.

In some embodiments R^{3a} is chlorine.

In some embodiments R^{3a} is C₁₋₆ linear alkyl.

In some embodiments R^{3a} is C₃₋₇ branched alkyl.

In some embodiments R^{3a} is C₁₋₆ linear alkoxy.

In some embodiments R^{3a} is C₃₋₇ branched alkoxy.

In some embodiments R^{3b} is hydrogen.

In some embodiments R^{3b} is fluorine.

In some embodiments R^{3b} is chlorine.

In some embodiments R^{3b} is C₁₋₆ linear alkyl.

In some embodiments R^{3b} is C₃₋₇ branched alkyl.

In some embodiments R^{3b} is C₁₋₆ linear alkoxy.

In some embodiments R^{3b} is C₃₋₇ branched alkoxy.

In some embodiments R^{3c} is C₁₋₆ linear alkyl.

In some embodiments R^{3c} is C₃₋₇ branched alkyl.

In some embodiments R^{3d} is hydrogen.

In some embodiments R^{3d} is fluorine.

In some embodiments R^{3d} is chlorine.

In some embodiments R^{3d} is C₁₋₆ linear alkyl.

In some embodiments R^{3d} is C₃₋₇ branched alkyl.

In some embodiments R^{3d} is C₁₋₆ linear alkoxy.

In some embodiments R^{3d} is C₃₋₇ branched alkoxy.

In some embodiments R^{3e} is hydrogen.

In some embodiments R^{3e} is fluorine.

In some embodiments R^{3e} is chlorine.

In some embodiments R^{3e} is C₁₋₆ linear alkyl.

In some embodiments R^{3e} is C₃₋₇ branched alkyl.

In some embodiments R^{3e} is C₁₋₆ linear alkoxy.

In some embodiments R^{3e} is C₃₋₇ branched alkoxy.

In some embodiments R^{3f} is hydrogen.

In some embodiments R^{3f} is CO₂R^{3g}.

In some embodiments R^{3f} is optionally substituted C₁₋₆ linear alkyl.

In some embodiments R^{3f} is optionally substitute C₃₋₇ branched alkyl.

In some embodiments R^{3f} is C₁₋₆ linear alkyl that is optionally substituted with a group selected from OH, CO₂H, and CO₂R^{3g}.

In some embodiments R^{3f} is C₃₋₇ branched alkyl that is optionally substituted with a group selected from OH, CO₂H, and CO₂R^{3g}.

In some embodiments R^{3g} is hydrogen.

In some embodiments R^{3g} is C₁₋₆ linear alkyl.

In some embodiments R^{3g} is C₃₋₇ branched alkyl.

In some embodiments R⁶ is hydrogen.

In some embodiments R⁶ is halogen.

In some embodiments R⁶ is optionally substituted C₁₋₆ linear alkyl.

In some embodiments R⁶ is optionally substituted C₃₋₇ branched alkyl.

In some embodiments R⁶ is optionally substituted C₃₋₈ cycloalkyl.

In some embodiments R⁶ is optionally substituted C₁₋₆ linear alkoxy.

In some embodiments R⁶ is optionally substituted C₃₋₇ branched alkoxy.

In some embodiments R⁶ is optionally substituted C₃₋₈ cycloalkoxy.

In some embodiments R⁶ is —OBenzyl.

In some embodiments R⁶ is

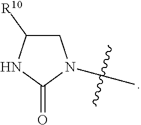

In some embodiments R⁶ is

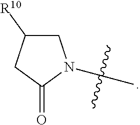

In some embodiments R⁶ is optionally substituted aryl.

In some embodiments R⁶ is a phenyl ring.

In some embodiments R⁶ is a phenyl ring that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁶ is a phenyl ring that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁶ is a phenyl ring that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁶ is optionally substituted heteroaryl.

In some embodiments R⁶ is a heteroaryl that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁶ is a heteroaryl that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁶ is a heteroaryl that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁷ is hydrogen.

In some embodiments R⁷ is optionally substituted C₁₋₆ linear alkyl.

In some embodiments R⁷ is optionally substituted C₃₋₇ branched alkyl.

In some embodiments R⁷ is C₁₋₆ fluoroalkyl.

In some embodiments R⁷ is C₃₋₇ branched fluoroalkyl.

In some embodiments R⁷ is optionally substituted C₁₋₆ alkenyl.

In some embodiments R⁷ is CO₂R¹⁰.

In some embodiments R⁷ is CONHR¹⁰.

In some embodiments R⁷ is SO₂R¹⁰.

In some embodiments R⁷ is

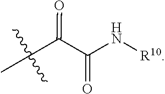

In some embodiments R^{7a} is hydrogen.

In some embodiments R^{7a} is optionally substituted C₁₋₆ linear alkyl.

In some embodiments R^{7a} is optionally substituted C₃₋₇ branched alkyl.

In some embodiments R^{7a} is C₁₋₆ fluoroalkyl.

In some embodiments R^{7a} is C₃₋₇ branched fluoroalkyl.

In some embodiments R^{7a} is optionally substituted C₁₋₆ alkenyl.

In some embodiments R^{7a} is CO₂R¹⁰.

In some embodiments R^{7a} is CONHR¹⁰.

In some embodiments R^{7a} is SO₂R¹⁰.

In some embodiments R^{7a} is

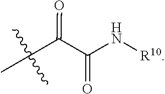

In some embodiments R^{7b} is hydrogen.

In some embodiments R^{7b} is optionally substituted C₁₋₆ linear alkyl.

In some embodiments R^{7b} is optionally substituted C₃₋₇ branched alkyl.

In some embodiments R^{7b} is C₁₋₆ fluoroalkyl.

In some embodiments R^{7b} is C₃₋₇ branched fluoroalkyl.

In some embodiments R^{7b} is optionally substituted C₁₋₆ alkenyl.

In some embodiments R^{7b} is CO₂R¹⁰.

In some embodiments R^{7b} is CONHR¹⁰.

In some embodiments R^{7b} is SO₂R¹⁰.

In some embodiments R^{7b} is

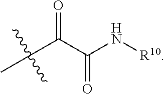

In some embodiments R⁸ is hydrogen.

In some embodiments R⁸ is halogen.

In some embodiments R⁸ is optionally substituted C₁₋₆ linear alkyl.

In some embodiments R⁸ is optionally substituted C₃₋₇ branched alkyl.

In some embodiments R⁸ is optionally substituted C₃₋₈ cycloalkyl.

In some embodiments R⁸ is optionally substituted aryl.

In some embodiments R⁸ is a phenyl ring.

In some embodiments R⁸ is a phenyl ring that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁸ is a phenyl ring that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁸ is a phenyl ring that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁸ is optionally substituted heteroaryl.

In some embodiments R⁸ is a heteroaryl that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁸ is a heteroaryl that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁸ is a heteroaryl that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R⁸ is CO₂R¹⁰.

In some embodiments R⁸ is CONHR¹⁰.

In some embodiments R⁸ is NHCOR¹⁰.

In some embodiments R⁸ is SO₂R¹⁰.

In some embodiments R⁸ is

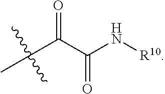

In some embodiments R⁹ is hydrogen.

In some embodiments R⁹ is optionally substituted C₁₋₄ alkyl.

In some embodiments R⁹ is optionally substituted halo C₁₋₄ alkyl.

In some embodiments R⁹ is optionally substituted C₃₋₇ cycloalkyl.

In some embodiments R¹⁰ is hydrogen.

In some embodiments R¹⁰ is optionally substituted C₁₋₆ linear alkyl.

In some embodiments R¹⁰ is optionally substituted C₃₋₇ branched alkyl.

In some embodiments R¹⁰ is optionally substituted C₃₋₈ cycloalkyl.

In some embodiments R¹⁰ is benzyl.

In some embodiments R¹⁰ is optionally substituted aryl.

In some embodiments R¹⁰ is phenyl.

In some embodiments R¹⁰ is a phenyl ring that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹⁰ is a phenyl ring that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹⁰ is a phenyl ring that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹⁰ is optionally substituted heteroaryl.

In some embodiments R¹⁰ is a heteroaryl that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹⁰ is a heteroaryl that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹⁰ is a heteroaryl that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹¹ is hydrogen.

In some embodiments R¹¹ is optionally substituted C₁₋₆ linear alkyl.

In some embodiments R¹¹ is optionally substituted C₃₋₇ branched alkyl.

In some embodiments R¹¹ is optionally substituted C₃₋₈ cycloalkyl.

In some embodiments R¹¹ is optionally substituted aryl.

In some embodiments R¹¹ is phenyl.

In some embodiments R¹¹ is a phenyl ring that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹¹ is a phenyl ring that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹¹ is a phenyl ring that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹¹ is optionally substituted heteroaryl.

In some embodiments R¹¹ is a heteroaryl that is substituted with 1 unit selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹¹ is a heteroaryl that is substituted with 2 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

In some embodiments R¹¹ is a heteroaryl that is substituted with 3 unit independently selected from group consisting of halogen, —CN, —NO₂, —OH, —NH₂, C₁₋₆ alkyl, C₃₋₇ branched alkyl, C₁₋₆ haloalkyl, C₁₋₆ alkoxy, C₁₋₆ haloalkoxy, C₂₋₈ alkenyl, and C₂₋₈ alkynyl, C₃₋₇ cycloalkyl, aryl, heterocycle, and heteroaryl.

For the purposes of the present invention, a compound depicted by the racemic formula will stand equally well for either of the two enantiomers having the formula or mixtures thereof, or in the case where a second chiral center is present, all diastereomers.

Exemplary embodiments include, but are not limited, to compounds of table 1:

TABLE 1

| Entry | Structure |
|---|---|
| 1 | 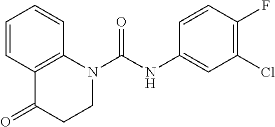 [SMILES: O=C1CCN(C(=O)Nc2ccc(F)c(Cl)c2)c2ccccc21] |
| 2 | 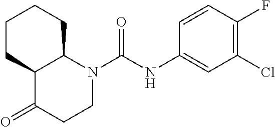 [SMILES: O=C1CCN(C(=O)Nc2ccc(F)c(Cl)c2)[C@@H]2CCCC[C@H]12] |
| 3 | 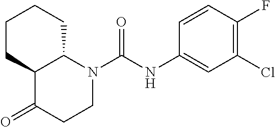 [SMILES: O=C1CCN(C(=O)Nc2ccc(F)c(Cl)c2)[C@H]2CCCC[C@H]12] |
| 4 | 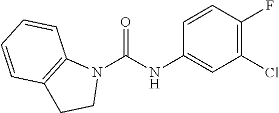 [SMILES: O=C(Nc1ccc(F)c(Cl)c1)N1CCc2ccccc21] |
| 5 | 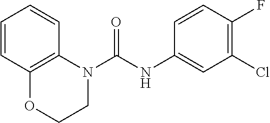 [SMILES: O=C(Nc1ccc(F)c(Cl)c1)N1CCOc2ccccc21] |
| 6 | 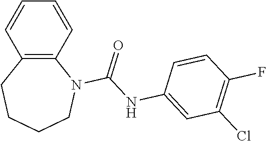 [SMILES: O=C(Nc1ccc(F)c(Cl)c1)N1CCCCc2ccccc21] |
| 7 | 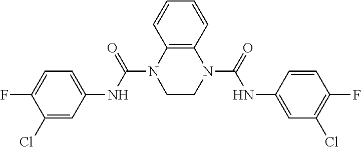 [SMILES: O=C(Nc1ccc(F)c(Cl)c1)N1CCN(C(=O)Nc2ccc(F)c(Cl)c2)c2ccccc21] |
| 8 | 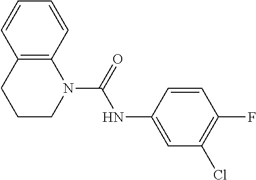 [SMILES: O=C(Nc1ccc(F)c(Cl)c1)N1CCCc2ccccc21] |
| 9 | 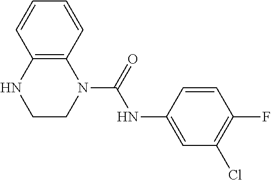 [SMILES: O=C(Nc1ccc(F)c(Cl)c1)N1CCNc2ccccc21] |
| 10 | 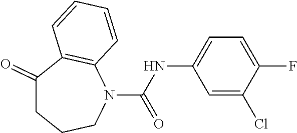 [SMILES: O=C1CCCN(C(=O)Nc2ccc(F)c(Cl)c2)c2ccccc21] |
| 11 | 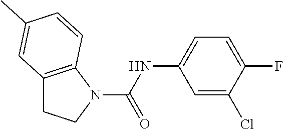 [SMILES: Cc1ccc2c(c1)CCN2C(=O)Nc1ccc(F)c(Cl)c1] |
| 12 | 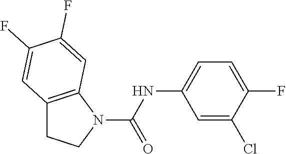 [SMILES: O=C(Nc1ccc(F)c(Cl)c1)N1CCc2cc(F)c(F)cc21] |
| 13 | 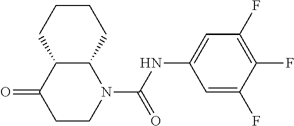 [SMILES: O=C1CCN(C(=O)Nc2cc(F)c(F)c(F)c2)[C@H]2CCCC[C@@H]12] |
| 14 | 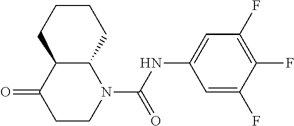 [SMILES: O=C1CCN(C(=O)Nc2cc(F)c(F)c(F)c2)[C@H]2CCCC[C@H]12] |
| 15 | 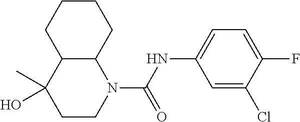 [SMILES: CC1(O)CCN(C(=O)Nc2ccc(F)c(Cl)c2)C2CCCCC21] |
| 16 | 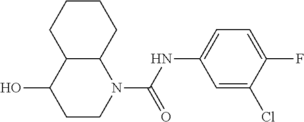 [SMILES: O=C(Nc1ccc(F)c(Cl)c1)N1CCC(O)C2CCCCC21] |
| 17 | 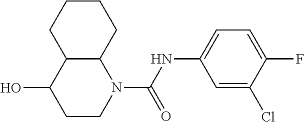 [SMILES: O=C(Nc1ccc(F)c(Cl)c1)N1CCC(O)C2CCCCC21] |
| 18 | 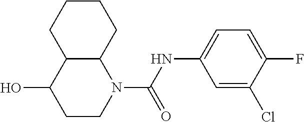 [SMILES: O=C(Nc1ccc(F)c(Cl)c1)N1CCC(O)C2CCCCC21] |
| 19 | 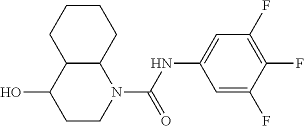 [SMILES: O=C(Nc1cc(F)c(F)c(F)c1)N1CCC(O)C2CCCCC21] |
| 20 | 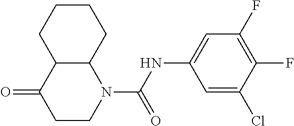 [SMILES: O=C1CCN(C(=O)Nc2cc(F)c(F)c(Cl)c2)C2CCCCC12] |
| 21 | 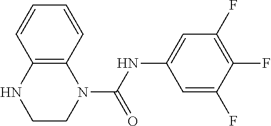 [SMILES: O=C(Nc1cc(F)c(F)c(F)c1)N1CCNc2ccccc21] |
| 22 | 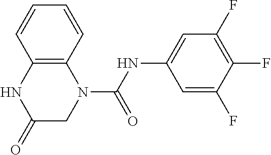 [SMILES: O=C1CN(C(=O)Nc2cc(F)c(F)c(F)c2)c2ccccc2N1] |
| 23 | 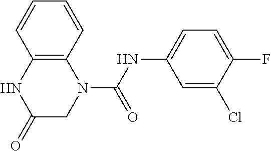 [SMILES: O=C1CN(C(=O)Nc2ccc(F)c(Cl)c2)c2ccccc2N1] |
| 24 | 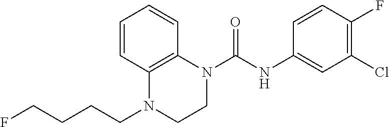 [SMILES: O=C(Nc1ccc(F)c(Cl)c1)N1CCN(CCCCF)c2ccccc21] |
| 25 | 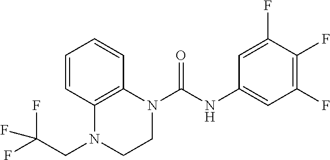 [SMILES: O=C(Nc1cc(F)c(F)c(F)c1)N1CCN(CC(F)(F)F)c2ccccc21] |
| 26 | 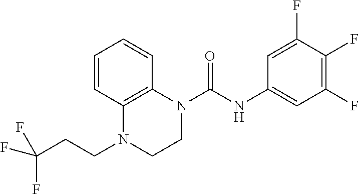 [SMILES: O=C(Nc1cc(F)c(F)c(F)c1)N1CCN(CCC(F)(F)F)c2ccccc21] |
| 27 | 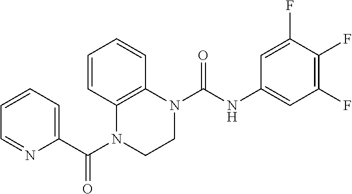 [SMILES: O=C(Nc1cc(F)c(F)c(F)c1)N1CCN(C(=O)c2ccccn2)c2ccccc21] |
| 28 | 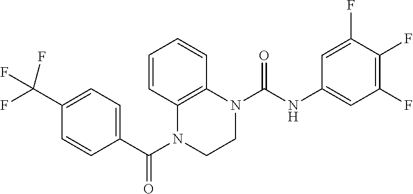 [SMILES: O=C(Nc1cc(F)c(F)c(F)c1)N1CCN(C(=O)c2ccc(C(F)(F)F)cc2)c2ccccc21] |
| 29 | 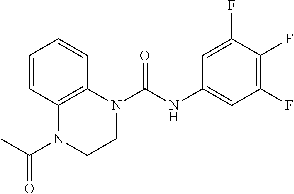 [SMILES: CC(=O)N1CCN(C(=O)Nc2cc(F)c(F)c(F)c2)c2ccccc21] |
| 30 | 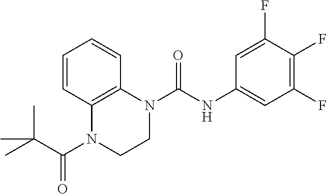 [SMILES: CC(C)(C)C(=O)N1CCN(C(=O)Nc2cc(F)c(F)c(F)c2)c2ccccc21] |
| 31 | 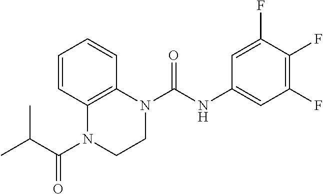 [SMILES: CC(C)C(=O)N1CCN(C(=O)Nc2cc(F)c(F)c(F)c2)c2ccccc21] |
| 32 | 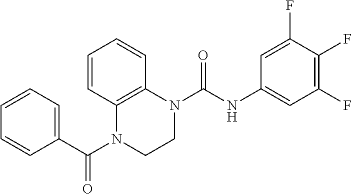 [SMILES: O=C(Nc1cc(F)c(F)c(F)c1)N1CCN(C(=O)c2ccccc2)c2ccccc21] |
| 33 | 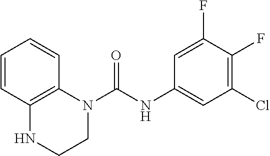 [SMILES: O=C(Nc1cc(F)c(F)c(Cl)c1)N1CCNc2ccccc21] |
| 34 | 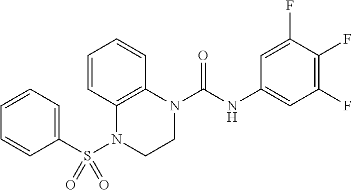 [SMILES: O=C(Nc1cc(F)c(F)c(F)c1)N1CCN(S(=O)(=O)c2ccccc2)c2ccccc21] |
| 35 | 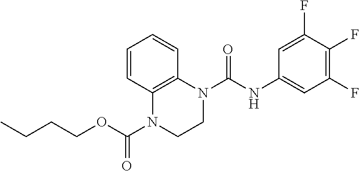 [SMILES: CCCCOC(=O)N1CCN(C(=O)Nc2cc(F)c(F)c(F)c2)c2ccccc21] |
| 36 | 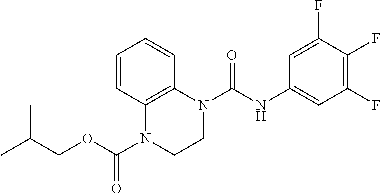 [SMILES: CC(C)COC(=O)N1CCN(C(=O)Nc2cc(F)c(F)c(F)c2)c2ccccc21] |
| 37 | 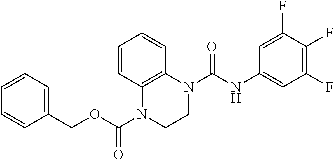 [SMILES: O=C(Nc1cc(F)c(F)c(F)c1)N1CCN(C(=O)OCc2ccccc2)c2ccccc21] |
| 38 | 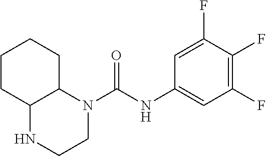 [SMILES: O=C(Nc1cc(F)c(F)c(F)c1)N1CCNC2CCCCC21] |
| 39 | 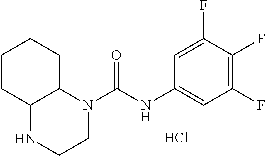 [SMILES: O=C(Nc1cc(F)c(F)c(F)c1)N1CCNC2CCCCC21 ; Cl] |
| 40 | 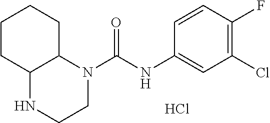 [SMILES: O=C(Nc1ccc(F)c(Cl)c1)N1CCNC2CCCCC21 ; Cl] |
| 41 | 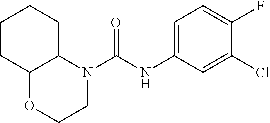 [SMILES: O=C(Nc1ccc(F)c(Cl)c1)N1CCOC2CCCCC21] |
| 42 | 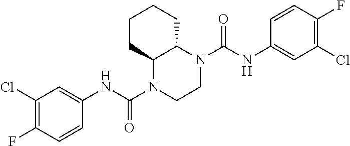 [SMILES: O=C(Nc1ccc(F)c(Cl)c1)N1CCN(C(=O)Nc2ccc(F)c(Cl)c2)[C@H]2CCCC[C@@H]21] |
| 43 | 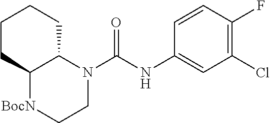 [SMILES: CC(C)(C)OC(=O)N1CCN(C(=O)Nc2ccc(F)c(Cl)c2)[C@H]2CCCC[C@@H]21] |
| 44 | 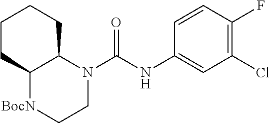 [SMILES: CC(C)(C)OC(=O)N1CCN(C(=O)Nc2ccc(F)c(Cl)c2)[C@@H]2CCCC[C@@H]21] |
| 45 | 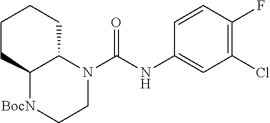 [SMILES: CC(C)(C)OC(=O)N1CCN(C(=O)Nc2ccc(F)c(Cl)c2)[C@H]2CCCC[C@@H]21] |
| 46 | 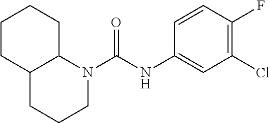 [SMILES: O=C(Nc1ccc(F)c(Cl)c1)N1CCCC2CCCCC21] |
| 47 | 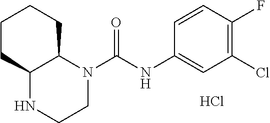 [SMILES: O=C(Nc1ccc(F)c(Cl)c1)N1CCN[C@H]2CCCC[C@H]21 ; Cl] |
| 48 | 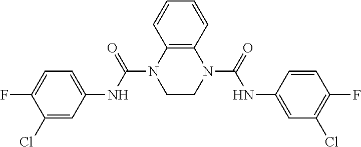 [SMILES: O=C(Nc1ccc(F)c(Cl)c1)N1CCN(C(=O)Nc2ccc(F)c(Cl)c2)c2ccccc21] |
| 49 | 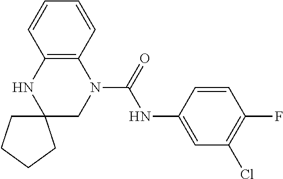 [SMILES: O=C(Nc1ccc(F)c(Cl)c1)N1CC2(CCCC2)Nc2ccccc21] |
| 50 | 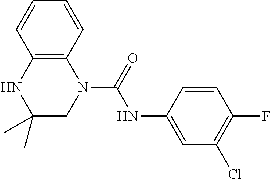 [SMILES: CC1(C)CN(C(=O)Nc2ccc(F)c(Cl)c2)c2ccccc2N1] |
| 51 | 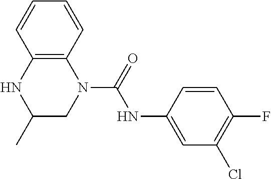 [SMILES: CC1CN(C(=O)Nc2ccc(F)c(Cl)c2)c2ccccc2N1] |
| 52 | 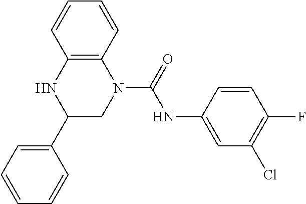 [SMILES: O=C(Nc1ccc(F)c(Cl)c1)N1CC(c2ccccc2)Nc2ccccc21] |
| 53 | 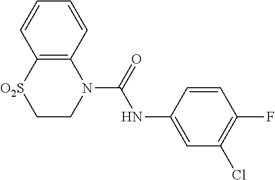 [SMILES: O=C(Nc1ccc(F)c(Cl)c1)N1CCS(=O)(=O)c2ccccc21] |
| 54 | 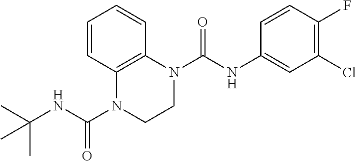 [SMILES: CC(C)(C)NC(=O)N1CCN(C(=O)Nc2ccc(F)c(Cl)c2)c2ccccc21] |
| 55 | 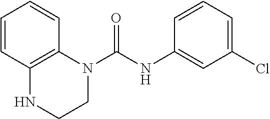 [SMILES: O=C(Nc1cccc(Cl)c1)N1CCNc2ccccc21] |
| 56 | 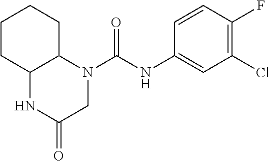 [SMILES: O=C1CN(C(=O)Nc2ccc(F)c(Cl)c2)C2CCCCC2N1] |
| 57 | 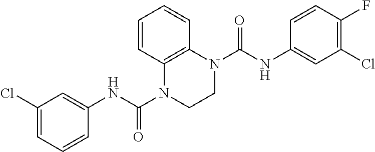 [SMILES: O=C(Nc1cccc(Cl)c1)N1CCN(C(=O)Nc2ccc(F)c(Cl)c2)c2ccccc21] |
| 58 | 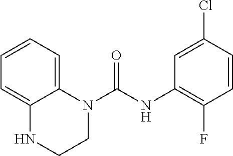 [SMILES: O=C(Nc1cc(Cl)ccc1F)N1CCNc2ccccc21] |
| 59 | 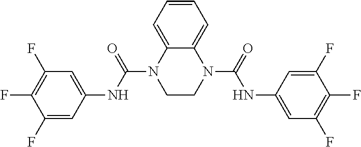 [SMILES: O=C(Nc1cc(F)c(F)c(F)c1)N1CCN(C(=O)Nc2cc(F)c(F)c(F)c2)c2ccccc21] |
| 60 | 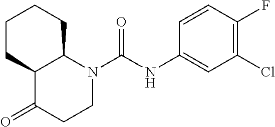 [SMILES: O=C1CCN(C(=O)Nc2ccc(F)c(Cl)c2)[C@@H]2CCCC[C@H]12] |
| 61 | 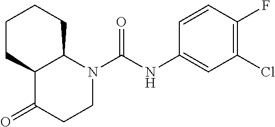 [SMILES: O=C1CCN(C(=O)Nc2ccc(F)c(Cl)c2)[C@@H]2CCCC[C@H]12] |
| 62 | 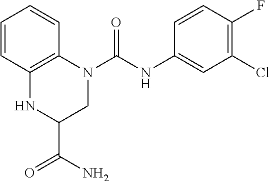 [SMILES: NC(=O)C1CN(C(=O)Nc2ccc(F)c(Cl)c2)c2ccccc2N1] |
| 63 | 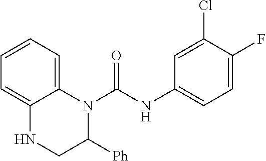 [SMILES: O=C(Nc1ccc(F)c(Cl)c1)N1c2ccccc2NCC1c1ccccc1] |
| 64 | 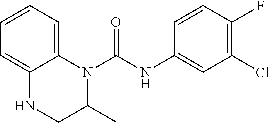 [SMILES: CC1CNc2ccccc2N1C(=O)Nc1ccc(F)c(Cl)c1] |
| 65 | 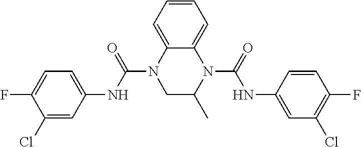 [SMILES: CC1CN(C(=O)Nc2ccc(F)c(Cl)c2)c2ccccc2N1C(=O)Nc1ccc(F)c(Cl)c1] |
| 66 | 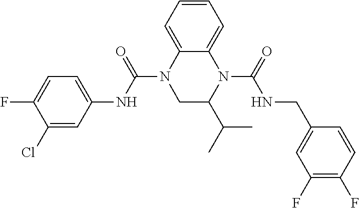 [SMILES: CC(C)C1CN(C(=O)Nc2ccc(F)c(Cl)c2)c2ccccc2N1C(=O)NCc1ccc(F)c(F)c1] |
| 67 | 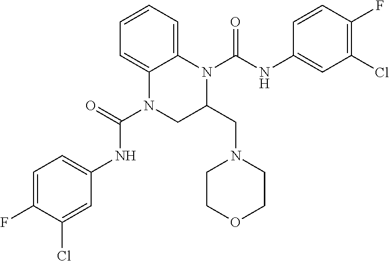 [SMILES: O=C(Nc1ccc(F)c(Cl)c1)N1CC(CN2CCOCC2)N(C(=O)Nc2ccc(F)c(Cl)c2)c2ccccc21] |
| 68 | 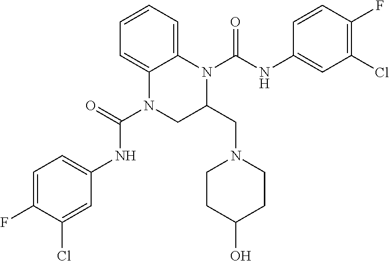 [SMILES: O=C(Nc1ccc(F)c(Cl)c1)N1CC(CN2CCC(O)CC2)N(C(=O)Nc2ccc(F)c(Cl)c2)c2ccccc21] |
| 69 | 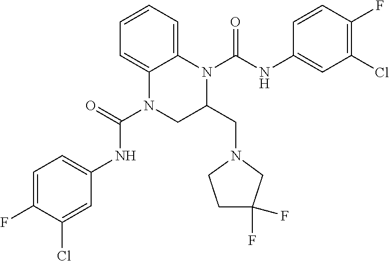 [SMILES: O=C(Nc1ccc(F)c(Cl)c1)N1CC(CN2CCC(F)(F)C2)N(C(=O)Nc2ccc(F)c(Cl)c2)c2ccccc21] |
| 70 | 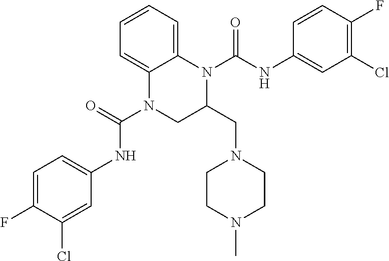 [SMILES: CN1CCN(CC2CN(C(=O)Nc3ccc(F)c(Cl)c3)c3ccccc3N2C(=O)Nc2ccc(F)c(Cl)c2)CC1] |
| 71 | 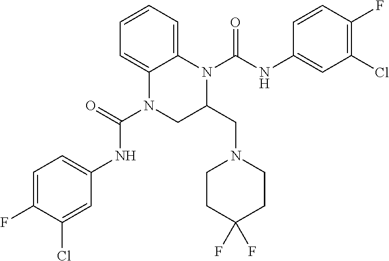 [SMILES: O=C(Nc1ccc(F)c(Cl)c1)N1CC(CN2CCC(F)(F)CC2)N(C(=O)Nc2ccc(F)c(Cl)c2)c2ccccc21] |
| 72 | 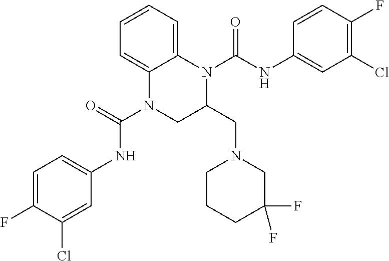 [SMILES: O=C(Nc1ccc(F)c(Cl)c1)N1CC(CN2CCCC(F)(F)C2)N(C(=O)Nc2ccc(F)c(Cl)c2)c2ccccc21] |
| 73 | 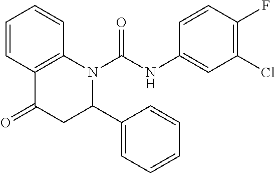 [SMILES: O=C1CC(c2ccccc2)N(C(=O)Nc2ccc(F)c(Cl)c2)c2ccccc21] |
| 74 | 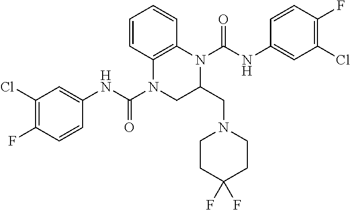 [SMILES: O=C(Nc1ccc(F)c(Cl)c1)N1CC(CN2CCC(F)(F)CC2)N(C(=O)Nc2ccc(F)c(Cl)c2)c2ccccc21] |
| 75 | 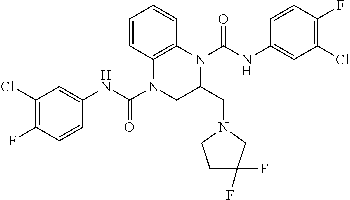 [SMILES: O=C(Nc1ccc(F)c(Cl)c1)N1CC(CN2CCC(F)(F)C2)N(C(=O)Nc2ccc(F)c(Cl)c2)c2ccccc21] |
| 76 | 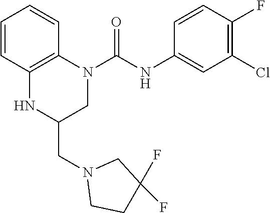 [SMILES: O=C(Nc1ccc(F)c(Cl)c1)N1CC(CN2CCC(F)(F)C2)Nc2ccccc21] |
| 77 | 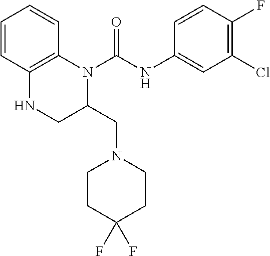 [SMILES: O=C(Nc1ccc(F)c(Cl)c1)N1c2ccccc2NCC1CN1CCC(F)(F)CC1] |
| 78 | 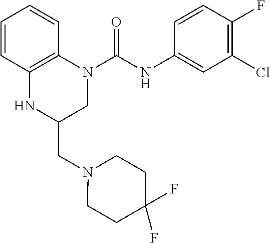 [SMILES: O=C(Nc1ccc(F)c(Cl)c1)N1CC(CN2CCC(F)(F)CC2)Nc2ccccc21] |
| 79 | 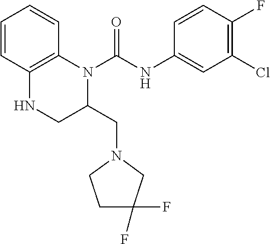 [SMILES: O=C(Nc1ccc(F)c(Cl)c1)N1c2ccccc2NCC1CN1CCC(F)(F)C1] |
| 80 | 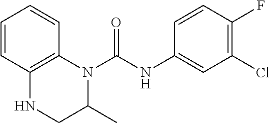 [SMILES: CC1CNc2ccccc2N1C(=O)Nc1ccc(F)c(Cl)c1] |
| 81 | 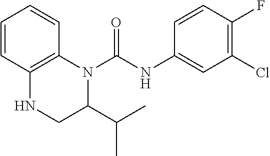 [SMILES: CC(C)C1CNc2ccccc2N1C(=O)Nc1ccc(F)c(Cl)c1] |
| 82 | 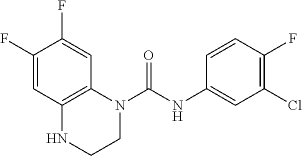 [SMILES: O=C(Nc1ccc(F)c(Cl)c1)N1CCNc2cc(F)c(F)cc21] |
| 83 | 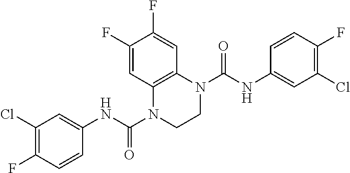 [SMILES: O=C(Nc1ccc(F)c(Cl)c1)N1CCN(C(=O)Nc2ccc(F)c(Cl)c2)c2cc(F)c(F)cc21] |
| 84 | 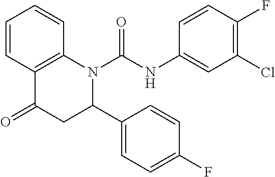 [SMILES: O=C1CC(c2ccc(F)cc2)N(C(=O)Nc2ccc(F)c(Cl)c2)c2ccccc21] |
| 85 | 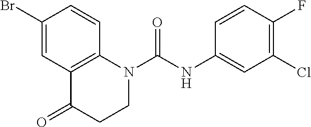 [SMILES: O=C1CCN(C(=O)Nc2ccc(F)c(Cl)c2)c2ccc(Br)cc21] |
| 86 | 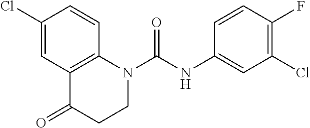 [SMILES: O=C1CCN(C(=O)Nc2ccc(F)c(Cl)c2)c2ccc(Cl)cc21] |
| 87 | 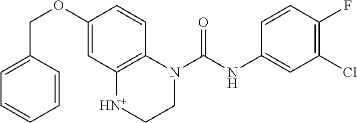 [SMILES: O=C(Nc1ccc(F)c(Cl)c1)N1CC[NH2+]c2cc(OCc3ccccc3)ccc21] |
| 88 | 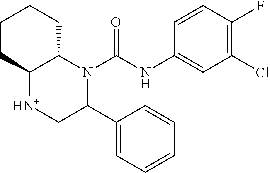 [SMILES: O=C(Nc1ccc(F)c(Cl)c1)N1C(c2ccccc2)C[NH2+][C@H]2CCCC[C@@H]21] |
| 89 | 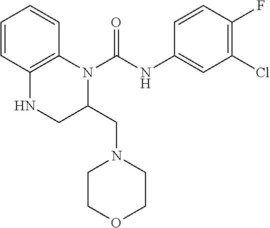 [SMILES: O=C(Nc1ccc(F)c(Cl)c1)N1c2ccccc2NCC1CN1CCOCC1] |
| 90 | 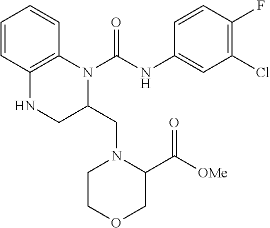 [SMILES: COC(=O)C1COCCN1CC1CNc2ccccc2N1C(=O)Nc1ccc(F)c(Cl)c1] |
| 91 | 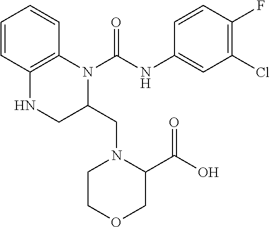 [SMILES: O=C(O)C1COCCN1CC1CNc2ccccc2N1C(=O)Nc1ccc(F)c(Cl)c1] |
| 92 | 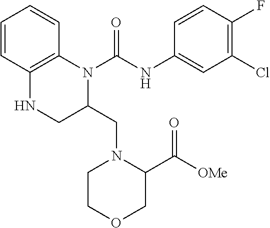 [SMILES: COC(=O)C1COCCN1CC1CNc2ccccc2N1C(=O)Nc1ccc(F)c(Cl)c1] |
| 93 | 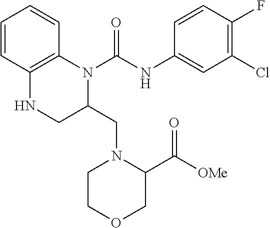 [SMILES: COC(=O)C1COCCN1CC1CNc2ccccc2N1C(=O)Nc1ccc(F)c(Cl)c1] |
| 94 | 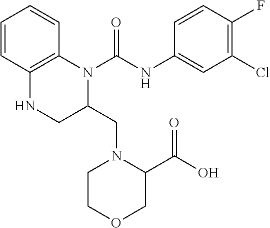 [SMILES: O=C(O)C1COCCN1CC1CNc2ccccc2N1C(=O)Nc1ccc(F)c(Cl)c1] |
| 95 | 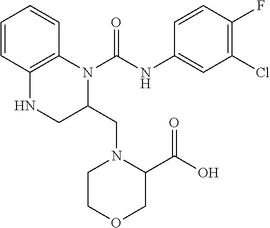 [SMILES: O=C(O)C1COCCN1CC1CNc2ccccc2N1C(=O)Nc1ccc(F)c(Cl)c1] |
| 96 | 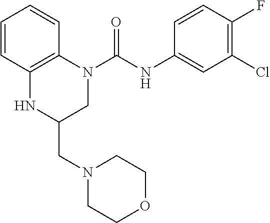 [SMILES: O=C(Nc1ccc(F)c(Cl)c1)N1CC(CN2CCOCC2)Nc2ccccc21] |
| 97 | 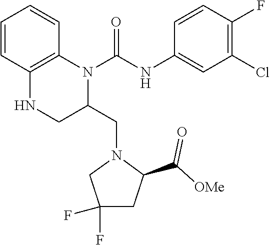 [SMILES: COC(=O)[C@H]1CC(F)(F)CN1CC1CNc2ccccc2N1C(=O)Nc1ccc(F)c(Cl)c1] |
| 98 | 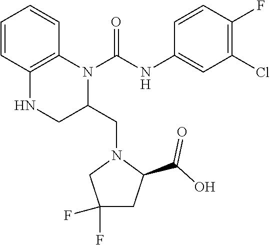 [SMILES: O=C(O)[C@H]1CC(F)(F)CN1CC1CNc2ccccc2N1C(=O)Nc1ccc(F)c(Cl)c1] |
| 99 | 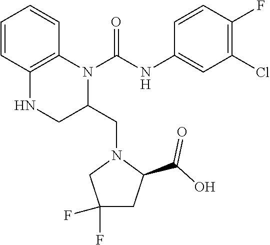 [SMILES: O=C(O)[C@H]1CC(F)(F)CN1CC1CNc2ccccc2N1C(=O)Nc1ccc(F)c(Cl)c1] |
| 100 | 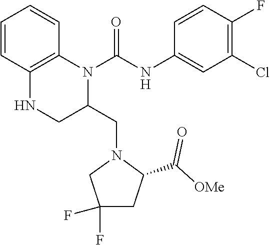 [SMILES: COC(=O)[C@@H]1CC(F)(F)CN1CC1CNc2ccccc2N1C(=O)Nc1ccc(F)c(Cl)c1] |
| 101 | 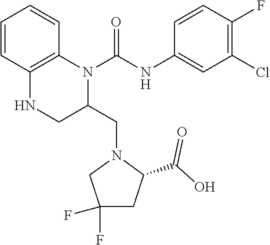 [SMILES: O=C(O)[C@@H]1CC(F)(F)CN1CC1CNc2ccccc2N1C(=O)Nc1ccc(F)c(Cl)c1] |
| 102 | 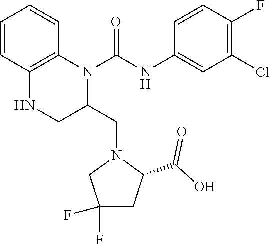 [SMILES: O=C(O)[C@@H]1CC(F)(F)CN1CC1CNc2ccccc2N1C(=O)Nc1ccc(F)c(Cl)c1] |
| 103 | 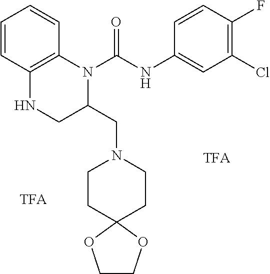 [SMILES: O=C(Nc1ccc(F)c(Cl)c1)N1c2ccccc2NCC1CN1CCC2(CC1)OCCO2] |
| 104 | 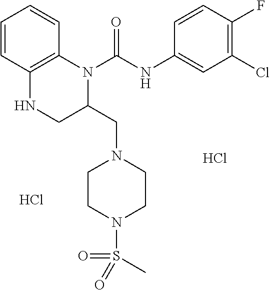 [SMILES: CS(=O)(=O)N1CCN(CC2CNc3ccccc3N2C(=O)Nc2ccc(F)c(Cl)c2)CC1 ; Cl ; Cl] |
| 105 | 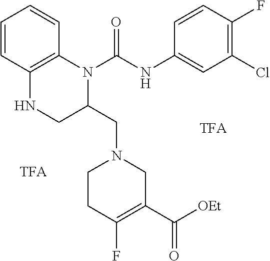 [SMILES: CCOC(=O)C1=C(F)CCN(CC2CNc3ccccc3N2C(=O)Nc2ccc(F)c(Cl)c2)C1] |
| 106 | 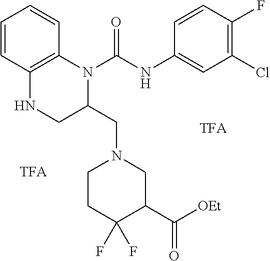 [SMILES: CCOC(=O)C1CN(CC2CNc3ccccc3N2C(=O)Nc2ccc(F)c(Cl)c2)CCC1(F)F] |
| 107 | 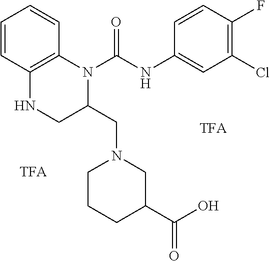 [SMILES: O=C(O)C1CCCN(CC2CNc3ccccc3N2C(=O)Nc2ccc(F)c(Cl)c2)C1] |
| 108 | 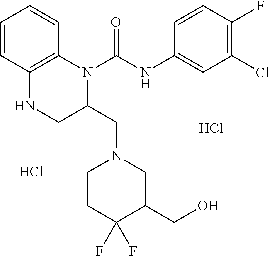 [SMILES: O=C(Nc1ccc(F)c(Cl)c1)N1c2ccccc2NCC1CN1CCC(F)(F)C(CO)C1 ; Cl ; Cl] |
| 109 | 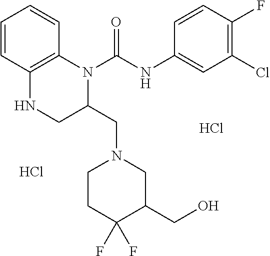 [SMILES: O=C(Nc1ccc(F)c(Cl)c1)N1c2ccccc2NCC1CN1CCC(F)(F)C(CO)C1 ; Cl ; Cl] |
| 110 | 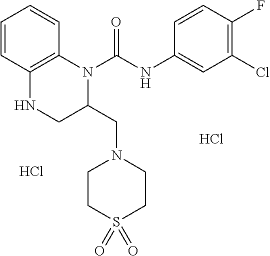 [SMILES: O=C(Nc1ccc(F)c(Cl)c1)N1c2ccccc2NCC1CN1CCS(=O)(=O)CC1 ; Cl ; Cl] |
| 111 | 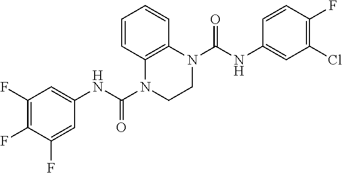 [SMILES: O=C(Nc1cc(F)c(F)c(F)c1)N1CCN(C(=O)Nc2ccc(F)c(Cl)c2)c2ccccc21] |
| 112 | 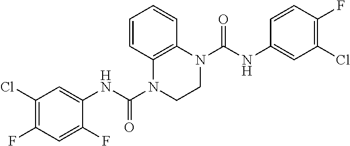 [SMILES: O=C(Nc1ccc(F)c(Cl)c1)N1CCN(C(=O)Nc2cc(Cl)c(F)cc2F)c2ccccc21] |
| 113 | 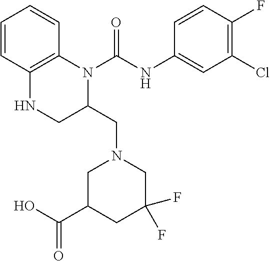 [SMILES: O=C(O)C1CN(CC2CNc3ccccc3N2C(=O)Nc2ccc(F)c(Cl)c2)CC(F)(F)C1] |
| 114 | 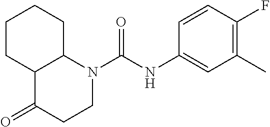 [SMILES: Cc1cc(NC(=O)N2CCC(=O)C3CCCCC32)ccc1F] |
| 115 | 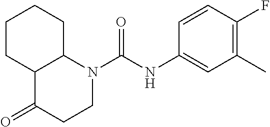 [SMILES: Cc1cc(NC(=O)N2CCC(=O)C3CCCCC32)ccc1F] |
| 116 | 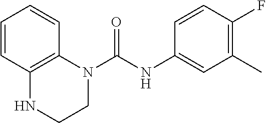 [SMILES: Cc1cc(NC(=O)N2CCNc3ccccc32)ccc1F] |
| 117 | 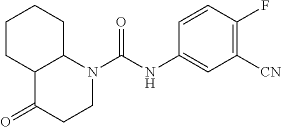 [SMILES: N#Cc1cc(NC(=O)N2CCC(=O)C3CCCCC32)ccc1F] |
| 118 | 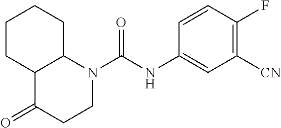 [SMILES: N#Cc1cc(NC(=O)N2CCC(=O)C3CCCCC32)ccc1F] |
| 119 | 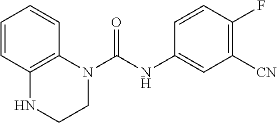 [SMILES: N#Cc1cc(NC(=O)N2CCNc3ccccc32)ccc1F] |
| 120 | 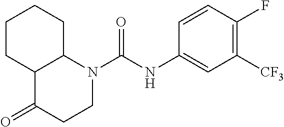 [SMILES: O=C1CCN(C(=O)Nc2ccc(F)c(C(F)(F)F)c2)C2CCCCC12] |
| 121 | 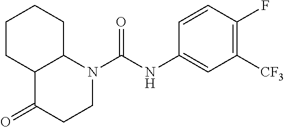 [SMILES: O=C1CCN(C(=O)Nc2ccc(F)c(C(F)(F)F)c2)C2CCCCC12] |
| 122 | 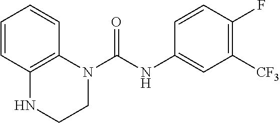 [SMILES: O=C(Nc1ccc(F)c(C(F)(F)F)c1)N1CCNc2ccccc21] |
| 123 | 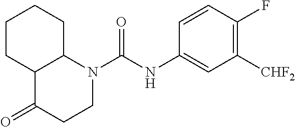 [SMILES: O=C1CCN(C(=O)Nc2ccc(F)c(C(F)F)c2)C2CCCCC12] |
| 124 | 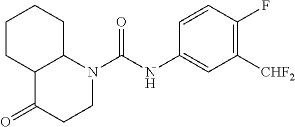 [SMILES: O=C1CCN(C(=O)Nc2ccc(F)c(C(F)F)c2)C2CCCCC12] |
| 125 | 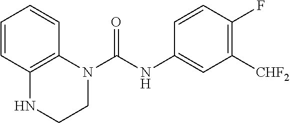 [SMILES: O=C(Nc1ccc(F)c(C(F)F)c1)N1CCNc2ccccc21] |
| 126 | 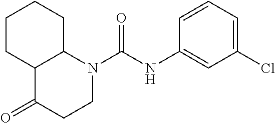 [SMILES: O=C1CCN(C(=O)Nc2cccc(Cl)c2)C2CCCCC12] |
| 127 | 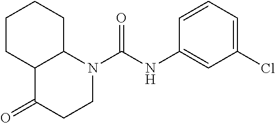 [SMILES: O=C1CCN(C(=O)Nc2cccc(Cl)c2)C2CCCCC12] |
| 128 | 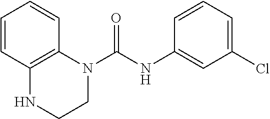 [SMILES: O=C(Nc1cccc(Cl)c1)N1CCNc2ccccc21] |

The compounds of the present invention include compounds having formula (II):

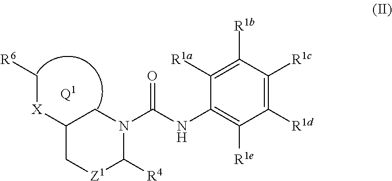

Including enantiomers, diasteromers, hydrates, solvates, pharmaceutically acceptable salts, prodrugs and complexes thereof.

For the purposes of demonstrating the manner in which the compounds of the present invention are named and referred to herein, the compound having the formula:

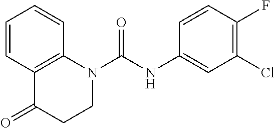 [SMILES: O=C1CCN(C(=O)Nc2ccc(F)c(Cl)c2)c2ccccc21]

has the chemical name N-(3-chloro-4-fluorophenyl)-4-oxo-3,4-dihydroquinoline-1(2H)-carboxamide.

For the purposes of demonstrating the manner in which the compounds of the present invention are named and referred to herein, the compound having the formula:

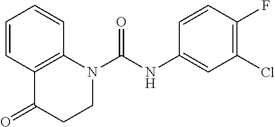 [SMILES: O=C1CCN(C(=O)Nc2ccc(F)c(Cl)c2)c2ccccc21]

has the chemical name N-(3-chloro-4-fluorophenyl)-4-oxo-3,4-dihydroquinoline-1(2H)-carboxamide.

For the purposes of demonstrating the manner in which the compounds of the present invention are named and referred to herein, the compound having the formula:

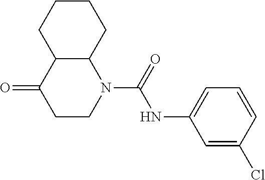 [SMILES: O=C1CCN(C(=O)Nc2cccc(Cl)c2)C2CCCCC12]

has the chemical name N-(3-chlorophenyl)-4-oxooctahydroquinoline-1(2H)-carboxamide.

For the purposes of the present invention, a compound depicted by the racemic formula will stand equally well for either of the two enantiomers or mixtures thereof, or in the case where a second chiral center is present, all diastereomers.

In all of the embodiments provided herein, examples of suitable optional substituents are not intended to limit the scope of the claimed invention. The compounds of the invention may contain any of the substituents, or combinations of substituents, provided herein.

Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatography such as high pressure liquid chromatography (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The compounds of these teachings can be prepared by methods known in the art of organic chemistry. The reagents used in the preparation of the compounds of these teachings can be either commercially obtained or can be prepared by standard procedures described in the literature. For example, compounds of the present invention can be prepared according to the method illustrated in the General Synthetic Schemes:

### General Synthetic Schemes for Preparation of Compounds.

The reagents used in the preparation of the compounds of this invention can be either commercially obtained or can be prepared by standard procedures described in the literature. In accordance with this invention, compounds in the genus may be produced by one of the following reaction schemes.

The first aspect of the process of the present invention relates to a process for preparing benzamides having the formula (I). Compounds of formula (I) may be prepared according to the process outlined in Schemes 1-x.

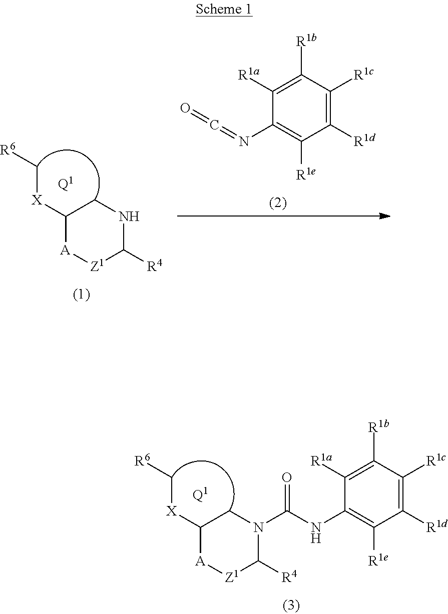

Accordingly, a suitably substituted compound of the formula (1), a known compound or compound prepared by known methods, is reacted with a compound of the formula (2), a known compound or a compound prepared by known methods, optionally in the presence an organic solvent such as methylene chloride, dichloroethane, tetrahydrofuran, 1,4-dioxane, N,N-dimethyl formamide, and the like, optionally with heating, optionally with microwave irradiation, to provide a compound of the formula (3).

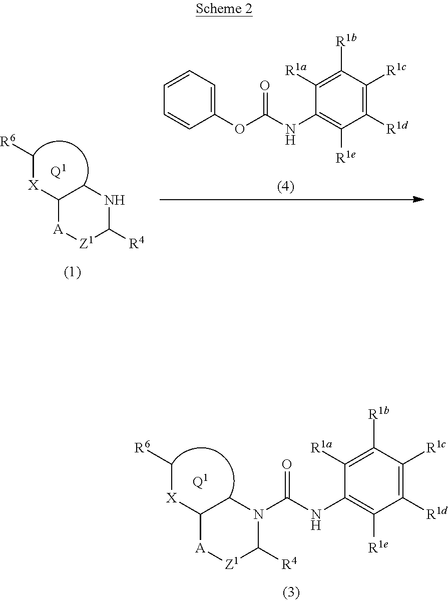

Alternatively, a suitably substituted compound of the formula (1), a known compound or compound prepared by known methods, is reacted with a compound of the formula (4), a known compound or compound prepared by known methods, in the presence of an organic solvent such as tetrahydrofuran, 1,4-dioxane, N,N-dimethylformamide, methylene chloride, dichloroethane, methanol, ethanol, and the like, optionally in the presence of a base such as triethylamine, diisopropylethylamine, pyridine, 2,6-lutidine, and the like, optionally in the presence of 4-N,N-dimethylaminopyridine, optionally with heating, optionally with microwave irradiation, to provide a compound of the formula (3).

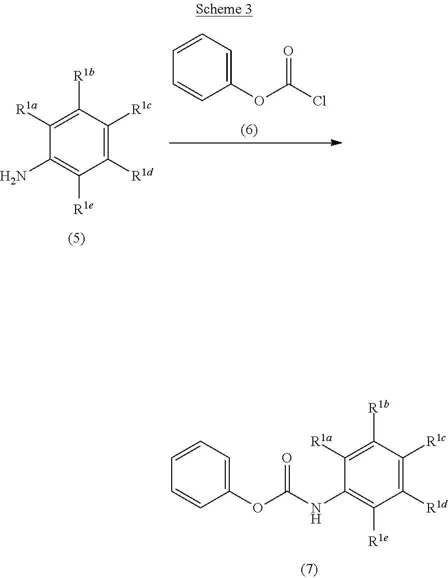

A compound of the formula (5), a known compound or a compound prepared by known methods, is reacted with a compound of the formula (6) in the presence of a base such as sodium bicarbonate, sodium carbonate, potassium bicarbonate, potassium carbonate, lithium bicarbonate, lithium carbonate, and the like, in a solvent such as ethyl acetate, acetonitrile tetrahydrofuran, 1,4-dioxane, N,N-dimethylformamide, methylene chloride, dichloroethane, optionally in the presence of water, optionally with heating, optionally with microwave irradiation, to provide a compound of the formula (7).

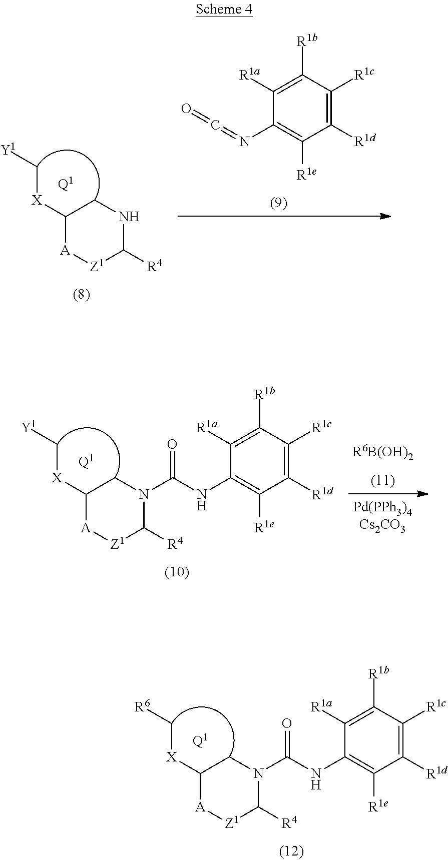

A compound of the formula (8), a known compound or a compound prepared by known methods wherein Y¹ is selected from the group consisting of bromine, chlorine, and methanetrifluorosulfonate, is reacted with a compound of the formula (6) a known compound or a compound prepared by known methods, optionally in the presence an organic solvent such as methylene chloride, dichloroethane, tetrahydrofuran, 1,4-dioxane, N,N-dimethyl formamide, and the like, optionally with heating, optionally with microwave irradiation, to provide a compound of the formula (10). A compound of the formula (10) is reacted with a compound of the formula (11), a known compound or a compound prepared by known methods, in the presence of a palladium catalyst such as palladium (II) acetate, tetrakis(triphenylphosphine)palladium(0), dichlorobis (triphenyl phosphine)palladium(II), bis(acetonitrile)dichloropalladium(II), tris(dibenzylideneacetone) dipalladium(0), and the like, in the presence of a base such as sodium carbonate, potassium carbonate, lithium carbonate, sodium bicarbonate, potassium bicarbonate, cesium carbonate. lithium bicarbonate, triethylamine, diisopropylethylamine, pyridine, and the like, optionally in the presence of water, in a solvent such as tetrahydrofuran, 1,4-dioxane, acetonitrile, N,N-dimethyl formamide, N,N-dimethylacetamide, methylene chloride, 1,2-dichloroethane, and the like, optionally with heating, optionally with microwave irradiation to provide a compound of the formula (12).

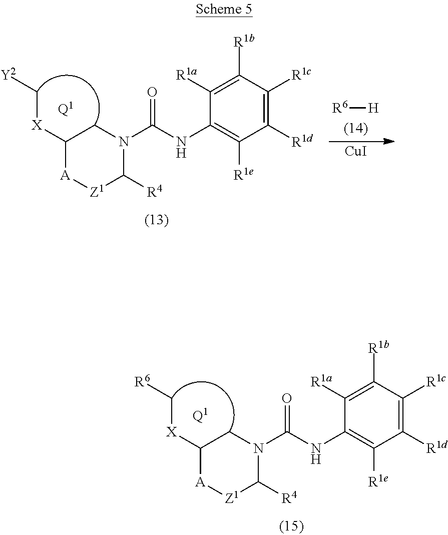

A compound of the formula (13), a known compound or a compound prepared by known methods wherein Y² is selected from the group consisting of bromine, chlorine, and methanetrifluorosulfonate, is reacted with a compound of the formula (14) a known compound or a compound prepared by known methods, in the presence of a copper iodide, in the presence of a base such as sodium carbonate, potassium carbonate, lithium carbonate, sodium bicarbonate, potassium bicarbonate, cesium carbonate. lithium bicarbonate, triethylamine, diisopropylethylamine, pyridine, and the like, optionally in a solvent such as tetrahydrofuran, 1,4-dioxane, acetonitrile, N,N-dimethyl formamide, N,N-dimethylacetamide, methylene chloride, 1,2-dichloroethane, and the like, optionally with heating, optionally with microwave irradiation to provide a compound of the formula (15).

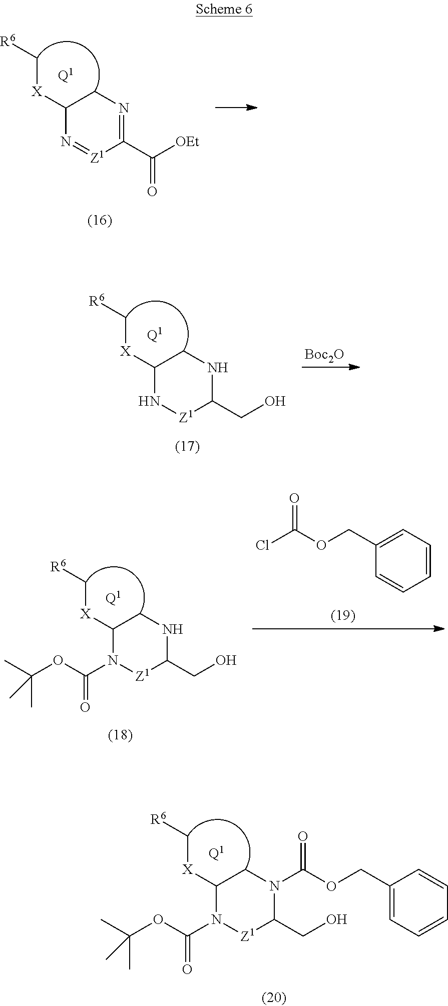

A compound of the formula (16), a known compound or a compound prepared by known methods, is reacted with lithium aluminum hydride in a solvent such as tetrahydrofuran, 1,4-dioxane, 1,2-dimethoxyethane, methylene chloride, 1,2-dichloroethane, and the like, optionally with heating, optionally with microwave irradiation, to provide a compound of the formula (17). A compound of the formula (17) is reacted with di-tert-butyl dicarbonate in the presence of a base such as such as pyridine, 2,6-lutidine, triethylamine, diisopropylethylamine, and the like, in a solvent such as methylene chloride, 1,2-dichloroethane, tetrahydrofuran, 1,4-dioxane, 1,2-dimethoxyethane, N,N-dimethylformamide, N,N-dimethylacetamide, methanol, ethanol, isopropanol, and the like, optionally with heating, optionally with microwave irradiation to provide a compound of the formula (18). A compound of the formula (18) is reacted with a compound of the formula (19), a known compound or a compound prepared by known methods, in the presence of a base such as triethylamine, diisopropylethylamine, pyridine, and the like, in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, and the like, optionally with heating, optionally with microwave irradiation to provide a compound of the formula (20). Alternatively, a compound of the formula (18) is reacted with a compound of the formula (19), a known compound or a compound prepared by known methods, in the presence of a base such sodium bicarbonate, sodium carbonate, potassium bicarbonate, potassium carbonate, lithium bicarbonate, lithium carbonate, and the like, optionally in the presence of water, in the presence of a solvent such as ethyl acetate, methylene chloride, 1,2-dichloroethane, tetrahydrofuran, 1,4-dioxane, 1,2-dimethoxyethane, and the like, optionally with heating, optionally with microwave irradiation to provide a compound of the formula (20).

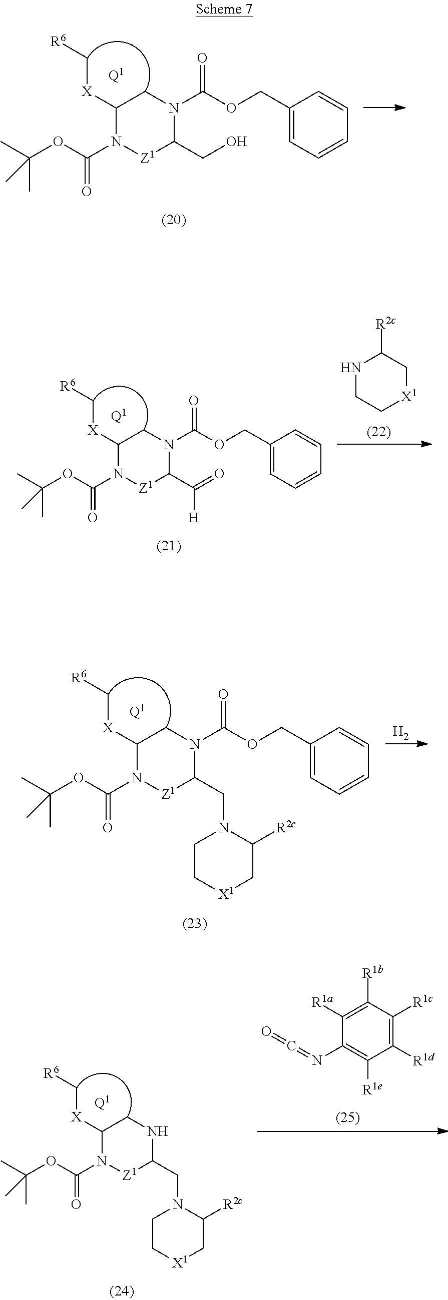

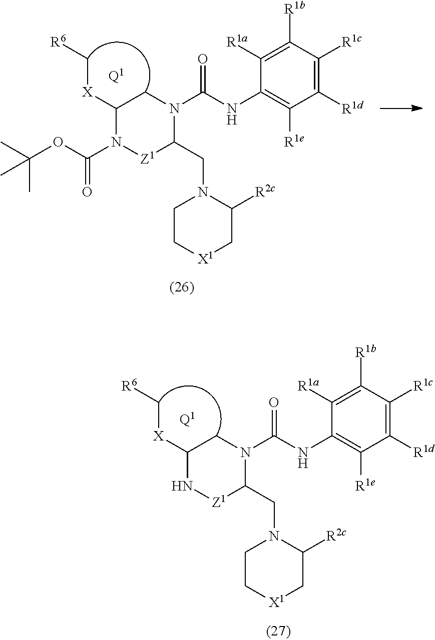

A compound of the formula (20) is reacted with a preformed mixture of dimethyl sulfoxide and oxalyl chloride and a base such as triethylamine, diisopropylethylamine, pyridine, and the like, in a solvent such as methylene chloride, 1,2-dichloroethane, 1,4-dioxane, tetrahydrofuran, 1,2-dimethoxytethane, and the like to provide a compound of the formula (21). A compound of the formula (21) is reacted with a compound of the formula (22), a known compound or a compound prepared by known methods, in the presence of a reducing agent such as sodium borohydride, sodium triacetoxy borohydride, sodium cyanoborohydride, lithium borohydride, lithium triacetoxy borohydride, lithium cyanoborohydride and the like, in the presence of a solvent such as methylene chloride, 1,2-dichloroethane, methanol, ethanol, isopropanol, tert-butanol, 1,4-dioxane, tetrahydrofuran, 1,2-dimethoxytethane, benzene toluene, N,N-dimethylformamide, N,N-dimethylacetamide, and the like, optionally in the presence of an acid such as acetic acid, trifluoroacetic acid, formic acid, and the like, optionally with heating, optionally with microwave irradiation to provide a compound of the formula (23). A compound of the formula (23) is reacted with hydrogen gas in the presence of a palladium catalyst such as palladium on carbon, palladium on barium sulfate, palladium (II) acetate, tetrakis(triphenylphosphine)palladium(0), dichlorobis (triphenylphosphine)palladium(II), palladium on carbon, bis(acetonitrile)dichloropalladium(II), and the like, in an organic solvent such as methanol, ethanol, ethyl acetate, tetrahydrofuran, 1,4-dioxane, dichloromethane, chloroform, 1,2-dichloroethane, N,N-dimethylformamide, and the like, to provide a compound of the formula (24). A compound of the formula (24) is reacted with a compound of the formula (25) a known compound or a compound prepared by known methods, optionally in the presence an organic solvent such as methylene chloride, dichloroethane, tetrahydrofuran, 1,4-dioxane, N,N-dimethyl formamide, and the like, optionally with heating, optionally with microwave irradiation, to provide a compound of the formula (26). A compound of the formula (26) is reacted with an acid such as trifluoroacetic acid, formic acid, acetic acid, hydrochloric acid, sulfuric acid, and the like, optionally in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, methanol, ethanol, isopropanol, and the like, optionally with heating, optionally with microwave irradiation to provide a compound of the formula (27).

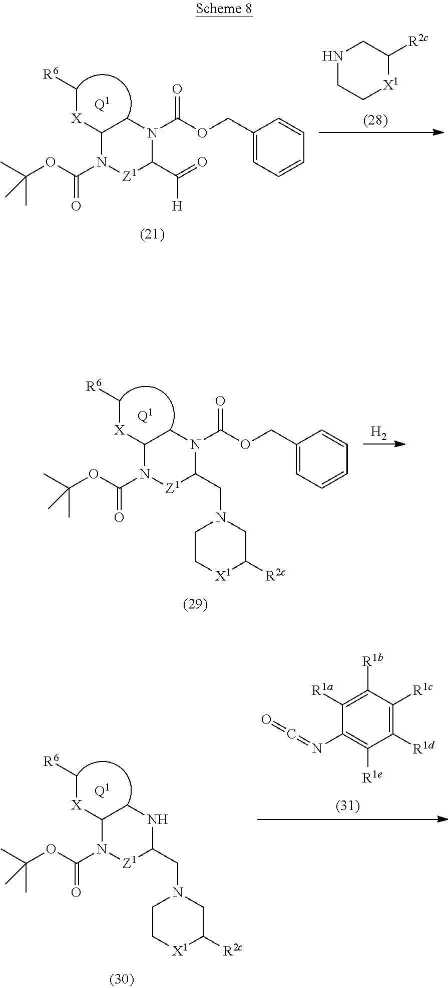

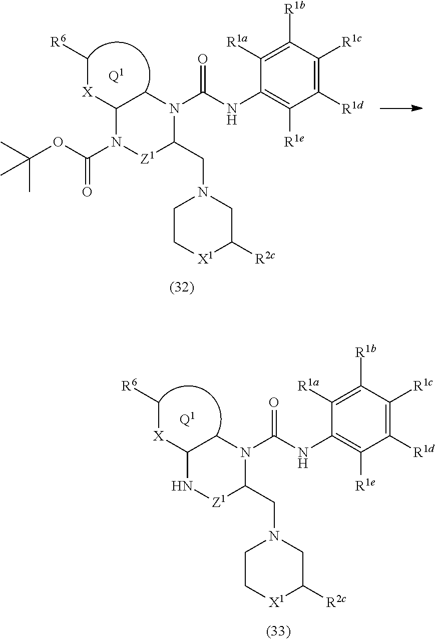

Alternatively, a compound of the formula (21) is reacted with a compound of the formula (28), a known compound or a compound prepared by known methods, in the presence of a reducing agent such as sodium borohydride, sodium triacetoxy borohydride, sodium cyanoborohydride, lithium borohydride, lithium triacetoxy borohydride, lithium cyanoborohydride and the like, in the presence of a solvent such as methylene chloride, 1,2-dichloroethane, methanol, ethanol, isopropanol, tert-butanol, 1,4-dioxane, tetrahydrofuran, 1,2-dimethoxtethane, benzene toluene, N,N-dimethylformamide, N,N-dimethylacetamide, and the like, optionally in the presence of an acid such as acetic acid, trifluoroacetic acid, formic acid, and the like, optionally with heating, optionally with microwave irradiation to provide a compound of the formula (29). A compound of the formula (29) is reacted with hydrogen gas in the presence of a palladium catalyst such as palladium on carbon, palladium on barium sulfate, palladium (II) acetate, tetrakis(triphenylphosphine)palladium(0), dichlorobis (triphenylphosphine)palladium(II), palladium on carbon, bis(acetonitrile)dichloropalladium(II), and the like, in an organic solvent such as methanol, ethanol, ethyl acetate, tetrahydrofuran, 1,4-dioxane, dichloromethane, chloroform, 1,2-dichloroethane, N,N-dimethylformamide, and the like, to provide a compound of the formula (30). A compound of the formula (30) is reacted with a compound of the formula (31) a known compound or a compound prepared by known methods, optionally in the presence an organic solvent such as methylene chloride, dichloroethane, tetrahydrofuran, 1,4-dioxane, N,N-dimethyl formamide, and the like, optionally with heating, optionally with microwave irradiation, to provide a compound of the formula (32). A compound of the formula (32) is reacted with an acid such as trifluoroacetic acid, formic acid, acetic acid, hydrochloric acid, sulfuric acid, and the like, optionally in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, methanol, ethanol, isopropanol, and the like, optionally with heating, optionally with microwave irradiation to provide a compound of the formula (33).

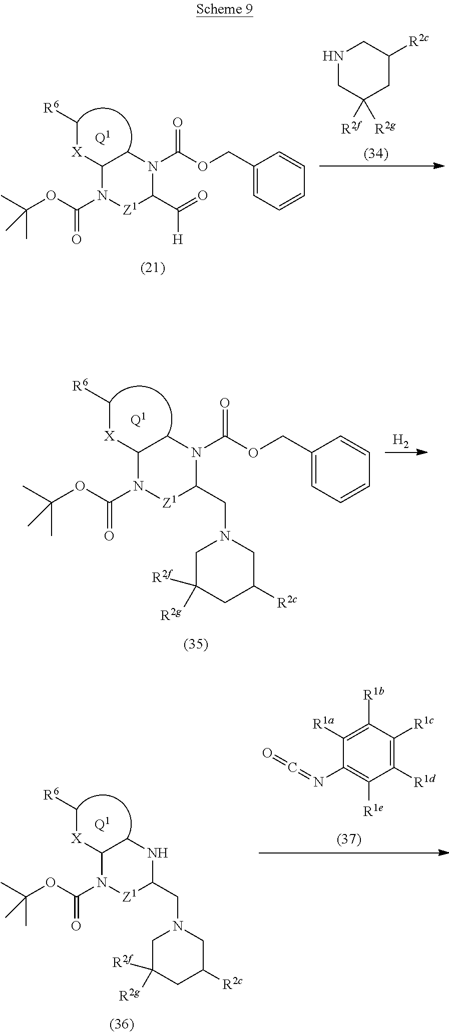

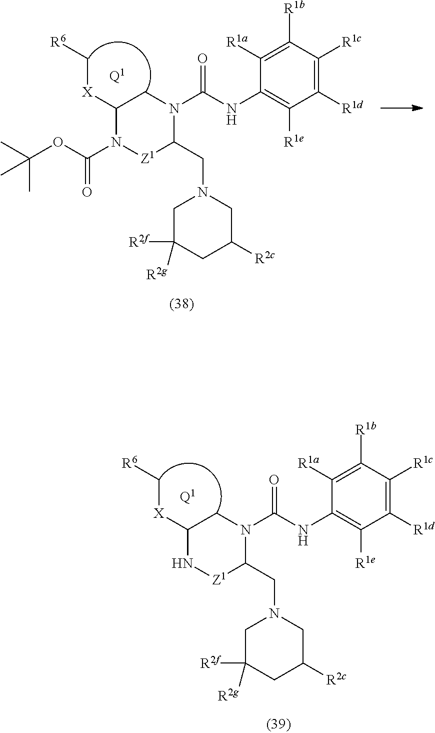

Alternatively, a compound of the formula (21) is reacted with a compound of the formula (34), a known compound or a compound prepared by known methods, in the presence of a reducing agent such as sodium borohydride, sodium triacetoxy borohydride, sodium cyanoborohydride, lithium borohydride, lithium triacetoxy borohydride, lithium cyanoborohydride and the like, in the presence of a solvent such as methylene chloride, 1,2-dichloroethane, methanol, ethanol, isopropanol, tert-butanol, 1,4-dioxane, tetrahydrofuran, 1,2-dimethoxtethane, benzene toluene, N,N-dimethylformamide, N,N-dimethylacetamide, and the like, optionally in the presence of an acid such as acetic acid, trifluoroacetic acid, formic acid, and the like, optionally with heating, optionally with microwave irradiation to provide a compound of the formula (35). A compound of the formula (35) is reacted with hydrogen gas in the presence of a palladium catalyst such as palladium on carbon, palladium on barium sulfate, palladium (II) acetate, tetrakis(triphenylphosphine)palladium(0), dichlorobis (triphenylphosphine)palladium(II), palladium on carbon, bis(acetonitrile)dichloropalladium(II), and the like, in an organic solvent such as methanol, ethanol, ethyl acetate, tetrahydrofuran, 1,4-dioxane, dichloromethane, chloroform, 1,2-dichloroethane, N,N-dimethylformamide, and the like, to provide a compound of the formula (36). A compound of the formula (36) is reacted with a compound of the formula (37) a known compound or a compound prepared by known methods, optionally in the presence an organic solvent such as methylene chloride, dichloroethane, tetrahydrofuran, 1,4-dioxane, N,N-dimethyl formamide, and the like, optionally with heating, optionally with microwave irradiation, to provide a compound of the formula (38). A compound of the formula (38) is reacted with an acid such as trifluoroacetic acid, formic acid, acetic acid, hydrochloric acid, sulfuric acid, and the like, optionally in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, methanol, ethanol, isopropanol, and the like, optionally with heating, optionally with microwave irradiation to provide a compound of the formula (39).

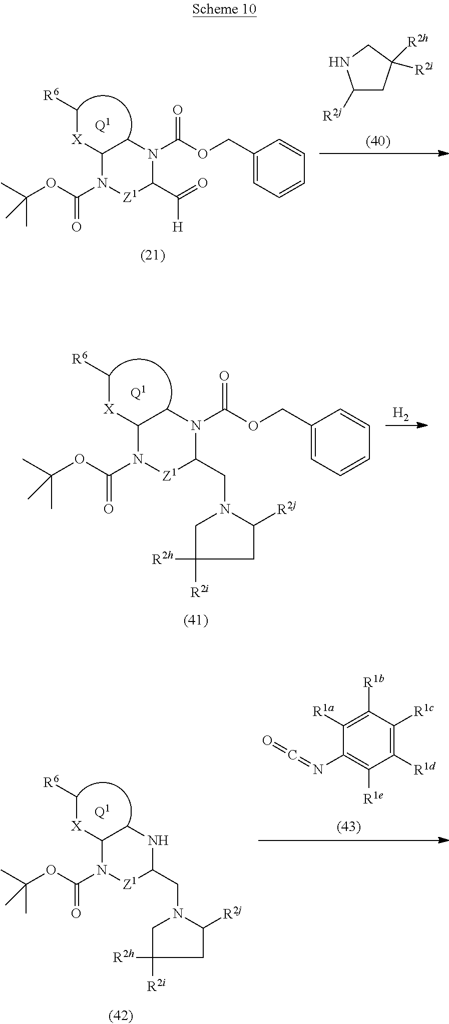

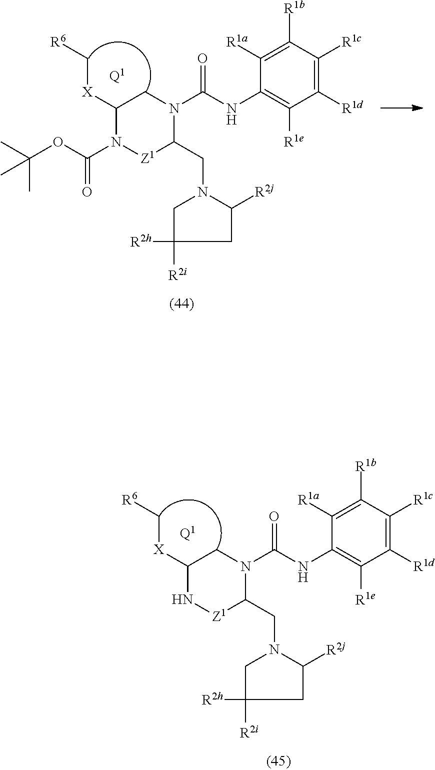

Alternatively, a compound of the formula (21) is reacted with a compound of the formula (40), a known compound or a compound prepared by known methods, in the presence of a reducing agent such as sodium borohydride, sodium triacetoxy borohydride, sodium cyanoborohydride, lithium borohydride, lithium triacetoxy borohydride, lithium cyanoborohydride and the like, in the presence of a solvent such as methylene chloride, 1,2-dichloroethane, methanol, ethanol, isopropanol, tert-butanol, 1,4-dioxane, tetrahydrofuran, 1,2-dimethoxtethane, benzene toluene, N,N-dimethylformamide, N,N-dimethylacetamide, and the like, optionally in the presence of an acid such as acetic acid, trifluoroacetic acid, formic acid, and the like, optionally with heating, optionally with microwave irradiation to provide a compound of the formula (41). A compound of the formula (35) is reacted with hydrogen gas in the presence of a palladium catalyst such as palladium on carbon, palladium on barium sulfate, palladium (II) acetate, tetrakis(triphenylphosphine)palladium(0), dichlorobis (triphenylphosphine)palladium(II), palladium on carbon, bis(acetonitrile)dichloropalladium(II), and the like, in an organic solvent such as methanol, ethanol, ethyl acetate, tetrahydrofuran, 1,4-dioxane, dichloromethane, chloroform, 1,2-dichloroethane, N,N-dimethylformamide, and the like, to provide a compound of the formula (42). A compound of the formula (42) is reacted with a compound of the formula (43) a known compound or a compound prepared by known methods, optionally in the presence an organic solvent such as methylene chloride, dichloroethane, tetrahydrofuran, 1,4-dioxane, N,N-dimethyl formamide, and the like, optionally with heating, optionally with microwave irradiation, to provide a compound of the formula (44). A compound of the formula (44) is reacted with an acid such as trifluoroacetic acid, formic acid, acetic acid, hydrochloric acid, sulfuric acid, and the like, optionally in a solvent such as methylene chloride, 1,2-dichloroethane, tetrahydrofuran, 1,4-dioxane, 1,2-dimethoxyethane, N,N-dimethylformamide, N,N-dimethylacetamide, acetonitrile, methanol, ethanol, isopropanol, and the like, optionally with heating, optionally with microwave irradiation to provide a compound of the formula (45).

### EXAMPLES

The examples below provide methods for preparing representative compounds of the disclosure. The skilled practitioner will know how to substitute the appropriate reagents, starting materials and purification methods known to those skilled in the art, in order to prepare additional compounds of the present invention.

¹H NMR spectra were recorded on a 300 MHz INOVA VARIAN spectrometer. Chemical shifts values are given in ppm and referred as the internal standard to TMS (tetramethylsilane). The peak patterns are indicated as follows: s, singlet; d, doublet; t, triplet; q, quadruplet; m, multiplet and dd, doublet of doublets. The coupling constants (J) are reported in Hertz (Hz). Mass Spectra were obtained on a 1200 Aligent LC-MS spectrometer (ES-API, Positive). Silica gel column chromatography was performed over silica gel 100-200 mesh, and the eluent was a mixture of ethyl acetate and hexanes, or mixture of methanol and ethyl acetate. All the tested compounds possess a purity of at least 95%. Analytical HPLC was run on the Agilent 1100 HPLC instrument, equipped with Agilent, ZORBAX SB-C18 column and UV detection at 210 nm.

### Example 1: Synthesis of phenyl (3-chloro-4-fluorophenyl)carbamate

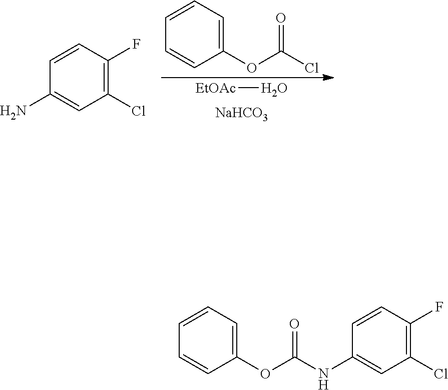

To a biphase mixture of ethyl acetate (5 ml) and saturated NaHCO₃ was added 3-chloro-4-fluoroaniline (123 mg, 0.84 mmol) and phenyl chloroformate (132 mg, 0.84 mmol) sequentially. The mixture was stirred for 10 minutes. The residue was purified on silica gel (12 g) with a gradient of ethyl acetate and hexane from 0:1 to 3:7 to provide a white solid (206 mg, 92%). Calculated for C₁₃H₉ClFNO₂, 265.0; observed LC/MS [M+H]=266.2.

### Example 2: Synthesis of N-(3-chloro-4-fluorophenyl)-4-oxo-3,4-dihydroquinoline-1(2H)-carboxamide

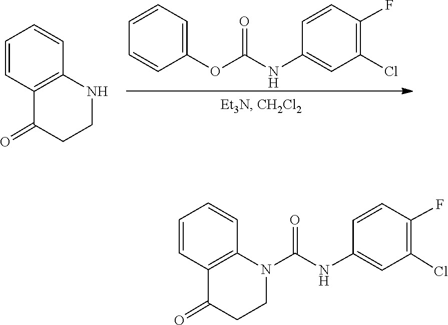 [SMILES: O=C1CCNc2ccccc21 ; O=C(Nc1ccc(F)c(Cl)c1)Oc1ccccc1 ; O=C1CCN(C(=O)Nc2ccc(F)c(Cl)c2)c2ccccc21]

To a solution of phenyl (3-chloro-4-fluorophenyl)carbamate (40 mg, 0.15 mmol) and triethylamine in CH₂Cl₂ (2 mL) was added 2,3-dihydroquinolin-4(1H)-one hydrochloride (28.0 mg, 0.15 mmol). The mixture was stirred at 23° C. for overnight. The reaction mixture was diluted with ethyl acetate and washed with HCl (2N) twice, saturated NaHCO₃, and brine. The organic phase was concentrated, and the residue was purified on HPLC, eluted with a gradient of acetonitrile and water from 30% to 100% to give the compound as a white solid (12 mg). Calculated for C₁₆H₁₂ClFN₂O₂, 318.1; observed LC/MS [M+H]=319.3.

### Example 3: Synthesis of N-(3-chloro-4-fluorophenyl)-4-oxooctahydroquinoline-1(2H)-carboxamide

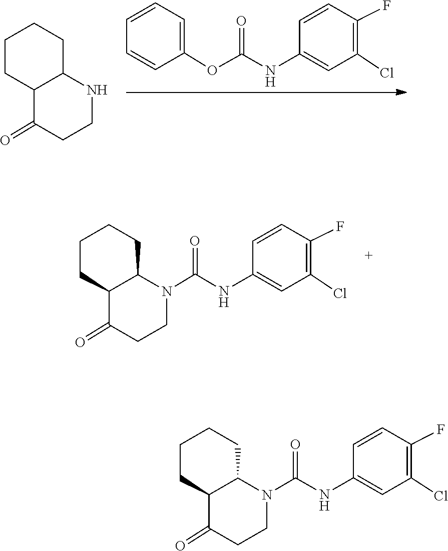 [SMILES: O=C1CCNC2CCCCC12 ; O=C(Nc1ccc(F)c(Cl)c1)Oc1ccccc1 ; O=C1CCN(C(=O)Nc2ccc(F)c(Cl)c2)[C@@H]2CCCC[C@H]12 ; O=C1CCN(C(=O)Nc2ccc(F)c(Cl)c2)[C@H]2CCCC[C@H]12]

The title compound was prepared according to the procedure of example 2 using octahydroquinolin-4(1H)-one and phenyl (3-chloro-4-fluorophenyl)carbamate to afford the cis and trans isomers of the title compound as off-white solids after silica gel separation with a gradient of ethyl acetate:hexanes from 0:1 to 3:7. Calculated for C₁₆H₁₈ClFN₂O₂, 324.1; observed LC/MS [M+H]=325.3.

### Example 4: Synthesis of N-(3-chloro-4-fluorophenyl)indoline-1-carboxamide

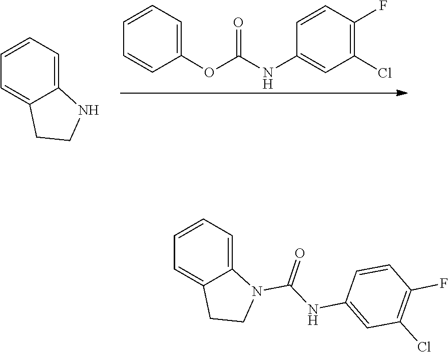 [SMILES: c1ccc2c(c1)CCN2 ; O=C(Nc1ccc(F)c(Cl)c1)Oc1ccccc1 ; O=C(Nc1ccc(F)c(Cl)c1)N1CCc2ccccc21]

The title compound was prepared according to the procedure of example 2 using indoline and phenyl (3-chloro-4-fluorophenyl)carbamate to afford the title compound as a light yellow solid after silica gel (12 g) separation with a gradient of ethyl acetate:hexanes from 0:1 to 4:6. Calculated for C₁₅H₁₂ClFN₂O, 290.1; observed LC/MS [M+H]=291.2.

### Example 5: Synthesis of N-(3-chloro-4-fluorophenyl)-2H-benzo[b][1,4]oxazine-4(3H)-carboxamide

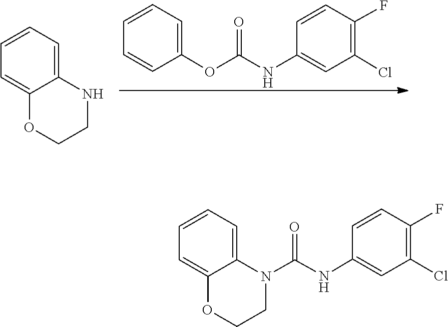 [SMILES: c1ccc2c(c1)NCCO2 ; O=C(Nc1ccc(F)c(Cl)c1)Oc1ccccc1 ; O=C(Nc1ccc(F)c(Cl)c1)N1CCOc2ccccc21]

The title compound was prepared according to the procedure of example 2 using 3,4-dihydro-2H-benzo[b][1,4]oxazine and phenyl (3-chloro-4-fluorophenyl)carbamate to afford the title compound as a light yellow solid after silica gel (12 g) separation with a gradient of ethyl acetate:hexanes from 0:1 to 2:8. Calculated for C₁₅H₁₂ClFN₂O₂, 306.0; observed LC/MS [M+H]=307.3.

### Example 6: Synthesis of N-(3-chlorophenyl)-4-oxooctahydroquinoline-1(2H)-carboxamide

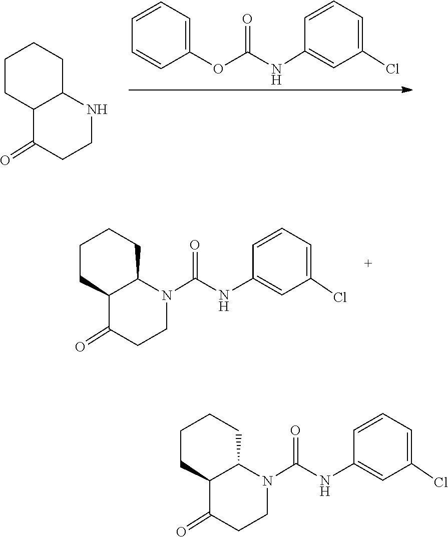 [SMILES: O=C1CCNC2CCCCC12 ; O=C(Nc1cccc(Cl)c1)Oc1ccccc1 ; O=C1CCN(C(=O)Nc2cccc(Cl)c2)[C@@H]2CCCC[C@H]12 ; O=C1CCN(C(=O)Nc2cccc(Cl)c2)[C@H]2CCCC[C@H]12]

The title compound was prepared according to the procedure of example 2 using octahydroquinolin-4(1H)-one and phenyl (3-chloro-phenyl)carbamate to afford the cis and trans isomers of the title compound as white solids after HPLC separation eluted with a gradient of acetonitrile and water from 30% to 100%. Calculated for C₁₆H₁₉ClN₂O₂, 306.1; observed LC/MS [M+H]=307.

### Example 7: Synthesis of N-(3-(difluoromethyl)-4-fluorophenyl)-4-oxooctahydro quinoline-1(2H)-carboxamide

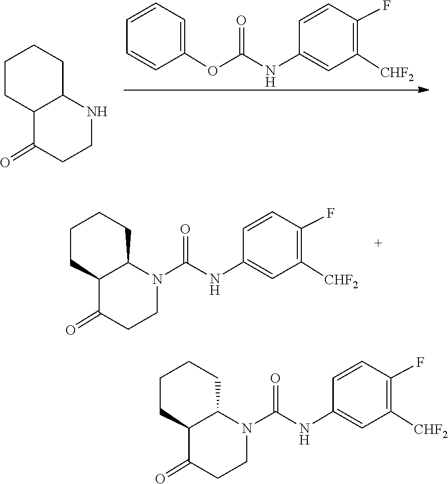 [SMILES: O=C1CCNC2CCCCC12 ; O=C(Nc1ccc(F)c(C(F)F)c1)Oc1ccccc1 ; O=C1CCN(C(=O)Nc2ccc(F)c(C(F)F)c2)[C@@H]2CCCC[C@H]12 ; O=C1CCN(C(=O)Nc2ccc(F)c(C(F)F)c2)[C@H]2CCCC[C@H]12]

The title compound was prepared according to the procedure of example 2 using octahydroquinolin-4(1H)-one and phenyl (3-(difluoromethyl)-4-fluorophenyl)carbamate to afford the cis and trans isomers of the title compound as white solids after HPLC separation eluted with a gradient of acetonitrile and water from 30% to 100%. Calculated for C₁₇H₁₉F₃N₂O₂, 340.1; observed LC/MS [M+H]=341.

### Example 8: Synthesis of N-(4-fluoro-3-(trifluoromethyl)phenyl)-4-oxooctahydro quinoline-1(2H)-carboxamide

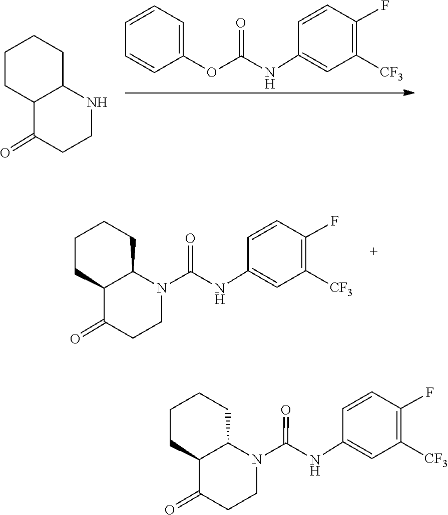 [SMILES: O=C1CCNC2CCCCC12 ; O=C(Nc1ccc(F)c(C(F)(F)F)c1)Oc1ccccc1 ; O=C1CCN(C(=O)Nc2ccc(F)c(C(F)(F)F)c2)[C@@H]2CCCC[C@H]12 ; O=C1CCN(C(=O)Nc2ccc(F)c(C(F)(F)F)c2)[C@H]2CCCC[C@H]12]

The title compound was prepared according to the procedure of example 2 using octahydroquinolin-4(1H)-one and phenyl (3-(trifluoromethyl)-4-fluorophenyl)carbamate to afford the cis and trans isomers of the title compound as white semi solids after HPLC separation eluted with a gradient of acetonitrile and water from 30% to 100%. Calculated for C₁₇H₁₈F₄N₂O₂, 358.1; observed LC/MS [M+H]=359.

### Example 9: Synthesis of N-(3-cyano-4-fluorophenyl)-4-oxooctahydroquinoline-1(2H)-carboxamide

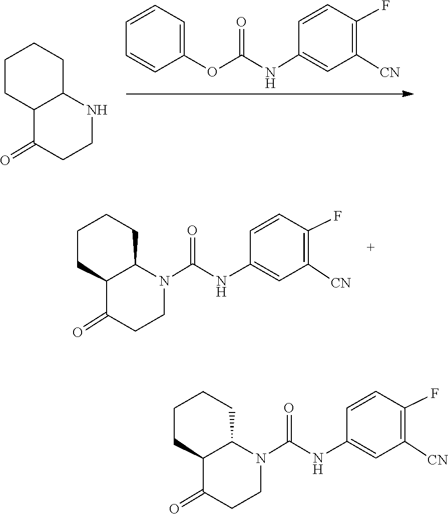 [SMILES: O=C1CCNC2CCCCC12 ; N#Cc1cc(NC(=O)Oc2ccccc2)ccc1F ; N#Cc1cc(NC(=O)N2CCC(=O)[C@H]3CCCC[C@H]32)ccc1F ; N#Cc1cc(NC(=O)N2CCC(=O)[C@H]3CCCC[C@@H]32)ccc1F]

The title compound was prepared according to the procedure of example 2 using octahydroquinolin-4(1H)-one and phenyl (3-cyano-4-fluorophenyl)carbamate to afford the cis and trans isomers of the title compound as pale pink semi solids after HPLC separation eluted with a gradient of acetonitrile and water from 30% to 100%. Calculated for C₁₇H₁₈FN₃O₂, 315.1; observed LC/MS [M+H]=316.

### Example 10: Synthesis of N-(4-fluoro-3-methylphenyl)-4-oxooctahydroquinoline-1(2H)-carboxamide

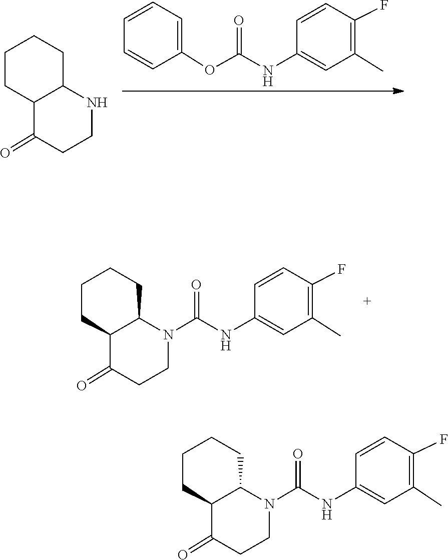 [SMILES: O=C1CCNC2CCCCC12 ; Cc1cc(NC(=O)Oc2ccccc2)ccc1F ; Cc1cc(NC(=O)N2CCC(=O)[C@H]3CCCC[C@H]32)ccc1F ; Cc1cc(NC(=O)N2CCC(=O)[C@H]3CCCC[C@@H]32)ccc1F]

The title compound was prepared according to the procedure of example 2 using octahydroquinolin-4(1H)-one and phenyl (3-methyl-4-fluorophenyl)carbamate to afford the cis and trans isomers of the title compound as pale yellow semi solids after HPLC separation eluted with a gradient of acetonitrile and water from 30% to 100%. Calculated for C₁₇H₂₁FN₂O₂, 304.2; observed LC/MS [M+H]=305.

### Example 11: Synthesis of N-(3-chloro-4-fluorophenyl)-3,4-dihydroquinoxaline-1(2H)-carboxamide and N1,N4-bis(3-chloro-4-fluorophenyl)-2,3-dihydroquinoxaline-1,4-dicarboxamide

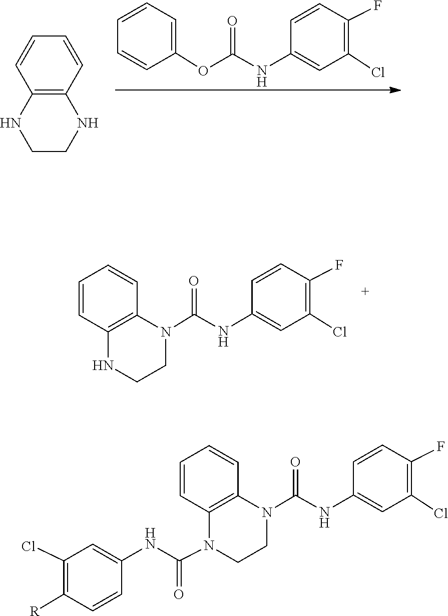

The title compounds were prepared according to the procedure of example 2 using 1,2,3,4-tetrahydroquinoxaline and phenyl (3-chloro-4-fluorophenyl)carbamate to afford N-(3-chloro-4-fluorophenyl)-3,4-dihydroquinoxaline-1(2H)-carboxamide and N1,N4-bis(3-chloro-4-fluorophenyl)-2,3-dihydroquinoxaline-1,4-dicarboxamide as white solids after HPLC separation eluted with a gradient of acetonitrile and water from 30% to 100%. Calculated for C₁₅H₁₃ClFN₃O, 305.1; observed LC/MS [M+H]=306; Calculated for C₂₂H₁₆Cl₂F₂N₄O₂, 476.1; observed LC/MS [M+H]=477.4.

### Example 12: Synthesis of N-(4-fluoro-3-methylphenyl)-3,4-dihydroquinoxaline-1(2H)-carboxamide

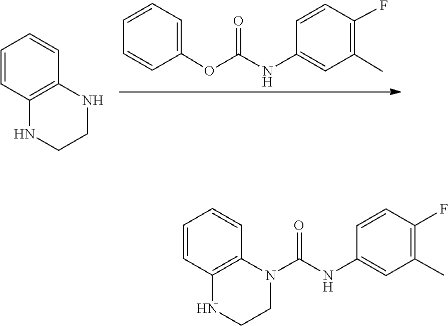 [SMILES: c1ccc2c(c1)NCCN2 ; Cc1cc(NC(=O)Oc2ccccc2)ccc1F ; Cc1cc(NC(=O)N2CCNc3ccccc32)ccc1F]

the title compound was prepared according to the procedure of example 2 using 1,2,3,4-tetrahydroquinoxaline and phenyl (3-methyl-4-fluorophenyl)carbamate to afford N-(3-methyl-4-fluorophenyl)-3,4-dihydroquinoxaline-1(2H)-carboxamide as a pale pink semi solid after HPLC separation eluted with a gradient of acetonitrile and water from 30% to 100%. Calculated for C₁₆H₁₆FN₃O, 285.1; observed LC/MS [M+H]=286.

### Example 13: Synthesis of N-(3-cyano-4-fluorophenyl)-3,4-dihydroquinoxaline-1(2H)-carboxamide

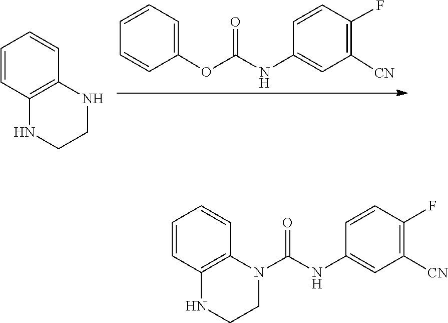 [SMILES: c1ccc2c(c1)NCCN2 ; N#Cc1cc(NC(=O)Oc2ccccc2)ccc1F ; N#Cc1cc(NC(=O)N2CCNc3ccccc32)ccc1F]

The title compound was prepared according to the procedure of example 2 using 1,2,3,4-tetrahydroquinoxaline and phenyl (3-cyano-4-fluorophenyl)carbamate to afford the desired product as light pink solid after HPLC separation eluted with a gradient of acetonitrile and water from 30% to 100%. Calculated for C₁₆H₁₃FN₄O, 296.1; observed LC/MS [M+H]=297.

### Example 14: Synthesis of N-(4-fluoro-3-(trifluoromethyl)phenyl)-3,4-dihydro quinoxaline-1(2H)-carboxamide

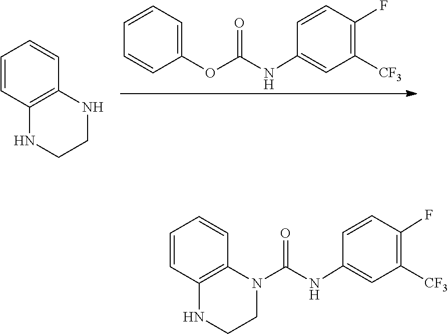 [SMILES: c1ccc2c(c1)NCCN2 ; O=C(Nc1ccc(F)c(C(F)(F)F)c1)Oc1ccccc1 ; O=C(Nc1ccc(F)c(C(F)(F)F)c1)N1CCNc2ccccc21]

The title compound was prepared according to the procedure of example 2 using 1,2,3,4-tetrahydroquinoxaline and phenyl (3-trifluoromethyl-4-fluorophenyl)carbamate to afford the title compound as light pink semisolid after HPLC separation eluted with a gradient of acetonitrile and water from 30% to 100%. Calculated for C₁₆H₁₃F₄N₃O, 339.1; observed LC/MS [M+H]=340.

### Example 15: Synthesis of N-(3-(difluoromethyl)-4-fluorophenyl)-3,4-dihydro quinoxaline-1(2H)-carboxamide

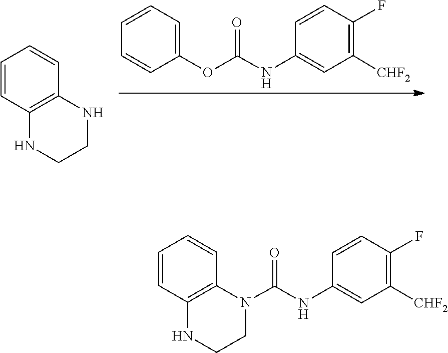 [SMILES: c1ccc2c(c1)NCCN2 ; O=C(Nc1ccc(F)c(C(F)F)c1)Oc1ccccc1 ; O=C(Nc1ccc(F)c(C(F)F)c1)N1CCNc2ccccc21]

The title compound was prepared according to the procedure of example 2 using 1,2,3,4-tetrahydroquinoxaline and phenyl (3-difluoromethyl-4-fluorophenyl)carbamate to afford the title compound as light pink semisolid after HPLC separation eluted with a gradient of acetonitrile and water from 30% to 100%. Calculated for C₁₆H₁₄F₃N₃O, 321.1; LC/MS [M+H]=observed 322.

### Example 16: Synthesis of N-(3-chlorophenyl)-3,4-dihydroquinoxaline-1(2H)-carboxamide

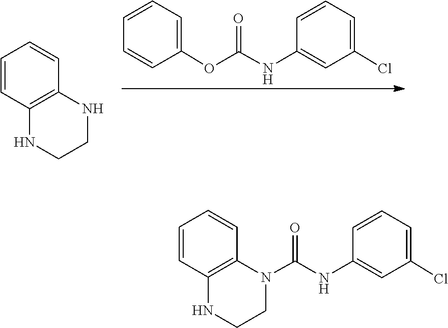 [SMILES: c1ccc2c(c1)NCCN2 ; O=C(Nc1cccc(Cl)c1)Oc1ccccc1 ; O=C(Nc1cccc(Cl)c1)N1CCNc2ccccc21]

The title compound was prepare according to the procedure of example 2 using 1,2,3,4-tetrahydroquinoxaline and phenyl (3-chlorophenyl)carbamate to afford desired product as light pink semisolid after HPLC separation eluted with a gradient of acetonitrile and water from 30% to 100%. Calculated for C₁₅H₁₄ClN₃O, 287.1; observed LC/MS [M+H]=288.

### Example 17: Synthesis of N-(3-chloro-4-fluorophenyl)-2-((4,4-difluoropiperidin-1-yl)methyl)-3,4-dihydroquinoxaline-1(2H)-carboxamide

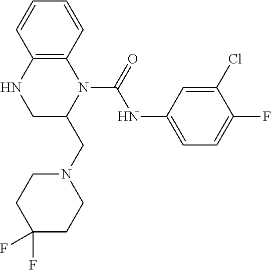 [SMILES: O=C(Nc1ccc(F)c(Cl)c1)N1c2ccccc2NCC1CN1CCC(F)(F)CC1]

### Step 1: Synthesis of tert-butyl 3-(hydroxymethyl)-3,4-dihydroquinoxaline-1(2H)-carboxylate

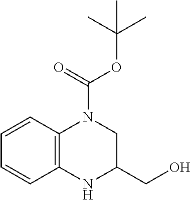 [SMILES: CC(C)(C)OC(=O)N1CC(CO)Nc2ccccc21]

Methyl quinoxaline-2-carboxylate (820 mg, 4.4 mmol) in tetrahydrofuran (15 ml) was treated with lithium aluminum hydride (2.4 M, 6 ml, 14.4 mmol) at 0° C. The mixture was stirred at room temperature for 4 hours, and then diluted with diethyl ether (30 ml), cooled to 0° C., and treated with Na₂SO₄ 10H₂O (10 g) for 18 hours. The mixture was filtered and the filtrate was treated with di-tert-butyl dicarbonate (2.2 g, 10 mmol) for 6 hours. The solvent was stripped under vacuum and the residual material was purified with silica gel (80 g) with a gradient of ethyl acetate:methylene chloride from 0:1 to 3:7 gave the desired isomer as a clear oil (663.5 mg, 57%).

### Step 2: Synthesis of 1-benzyl 4-tert-butyl 2-(hydroxymethyl)-2,3-dihydroquinoxaline-1,4-dicarboxylate

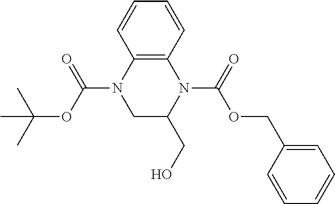 [SMILES: CC(C)(C)OC(=O)N1CC(CO)N(C(=O)OCc2ccccc2)c2ccccc21]

tert-Butyl 3-(hydroxymethyl)-3,4-dihydroquinoxaline-1(2H)-carboxylate (1.56 g, 5.91 mmol) was dissolved in ethyl acetate (20 ml), treated with saturated NaHCO₃ (10 ml) and benzyl chloroformate (1.1 ml, 7.9 mmol) sequentially. The mixture was stirred at 23° C. for 18 hours. The organic phase was concentrated, and the residue was purified on silica gel (120 g) with a gradient of ethyl acetate:methylene chloride from 0:1 to 2:8 gave the desired product as a sticky foam (1.64 g, 70%).

### Step 3: Synthesis of 1-benzyl 4-tert-butyl 2-formyl-2,3-dihydroquinoxaline-1,4-dicarboxylate

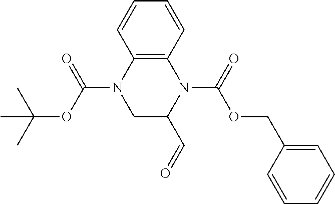 [SMILES: CC(C)(C)OC(=O)N1CC(C=O)N(C(=O)OCc2ccccc2)c2ccccc21]

To a stirred solution of oxalyl chloride (0.15 ml, 1.73 mmol) in CH₂Cl₂ (6 ml) at −78° C. was added dimethylsulfoxide (0.18 ml, 2.61 mmol). The solution was stirred for 5 minutes, followed with addition of 1-benzyl 4-tert-butyl 2-formyl-2,3-dihydroquinoxaline-1,4-dicarboxylate (345 mg, 0.87 mmol) in CH₂Cl₂ (2 ml). The mixture was stirred at this temperature for 1 hour. Then triethylamine (0.49 ml, 3.48 mmol) was added. The cooling bath was removed and the mixture was stirred at 23° C. for 18 hours. The mixture was diluted with ethyl acetate, washed with saturated NH₄Cl and brine. The organic phase was concentrated and purified on silica gel (40 g) with a gradient of ethyl acetate:methylene chloride from 0:1 to 1:9 gave the desired product as a light yellow oil (290 mg, 85%).

### Step 4: Synthesis of 1-benzyl 4-tert-butyl 2-((4,4-difluoropiperidin-1-yl)methyl)-2,3-dihydroquinoxaline-1,4-dicarboxylate

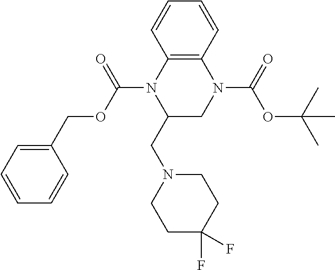 [SMILES: CC(C)(C)OC(=O)N1CC(CN2CCC(F)(F)CC2)N(C(=O)OCc2ccccc2)c2ccccc21]

1-benzyl 4-tert-butyl 2-formyl-2,3-dihydroquinoxaline-1,4-dicarboxylate was treated with 4,4-difluoropiperidine and triacetoxyborohydride in 1,2 dichloroethane at 23° C. The mixture was stirred overnight, and then quenched with saturated NaHCO3. The organic phase was concentrated and the residue was purified on silica gel to provide the desired product as a clear oil.

### Step 5: Synthesis of tert-butyl 3-((4,4-difluoropiperidin-1-yl)methyl)-3,4-dihydro quinoxaline-1(2H)-carboxylate

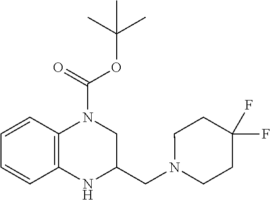 [SMILES: CC(C)(C)OC(=O)N1CC(CN2CCC(F)(F)CC2)Nc2ccccc21]

The 1-benzyl 4-tert-butyl 2-((4,4-difluoropiperidin-1-yl)methyl)-2,3-dihydroquinoxaline-1,4-dicarboxylate obtained above was treated with hydrogen in ethanol under Pd/C at 1 atm for overnight. Filtration and removal of the solvents under vacuum afforded the desired product as a light yellow oil.

### Step 6: Synthesis of tert-butyl 4-((3-chloro-4-fluorophenyl)carbamoyl)-3-((4,4-difluoropiperidin-1-yl)methyl)-3,4-dihydroquinoxaline-1(2H)-carboxylate

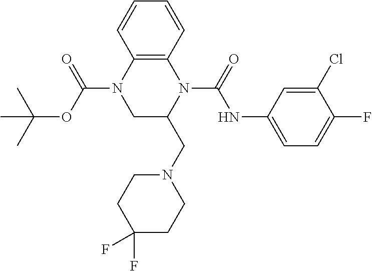 [SMILES: CC(C)(C)OC(=O)N1CC(CN2CCC(F)(F)CC2)N(C(=O)Nc2ccc(F)c(Cl)c2)c2ccccc21]

According to the general scheme 1, tert-butyl 3-((4,4-difluoropiperidin-1-yl)methyl)-3,4-dihydroquinoxaline-1(2H)-carboxylate in THF was treated with 2-chloro-1-fluoro-4-isocyanatobenzene to generate the desired product after HPLC separation.

### Step 7: Synthesis of N-(3-chloro-4-fluorophenyl)-2-((4,4-difluoropiperidin-1-yl) methyl)-3,4-dihydroquinoxaline-1(2H)-carboxamide

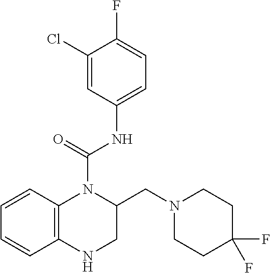 [SMILES: O=C(Nc1ccc(F)c(Cl)c1)N1c2ccccc2NCC1CN1CCC(F)(F)CC1]

The tert-butyl 4-((3-chloro-4-fluorophenyl)carbamoyl)-3-((4,4-difluoropiperidin-1-yl)methyl)-3,4-dihydroquinoxaline-1(2H)-carboxylate was dissolved in methanol (1 ml) and treated with HCl (4M in dioxane, 1 ml) for 4 hours at 23° C. to provide the desired product as a pale pink solid. Calculated for C₂₁H₂₂ClF₃N₄O, 438.1; observed LC/MS [M+H]=439.5.

### Formulations

The present invention also relates to compositions or formulations which comprise the pregenomic RNA encapsidation inhibitors according to the present invention. In general, the compositions of the present invention comprise an effective amount of one or more functionalized benzamide derivatives and salts thereof according to the present invention which are effective for useful for the treatment of Hepatitis B virus (HBV) infection and related conditions; and one or more excipients.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known antiviral agents. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Non-limiting examples of compositions according to the present invention include from about 0.001 mg to about 1000 mg of one or more pregenomic RNA encapsidation inhibitors according to the present invention and one or more excipients; from about 0.01 mg to about 100 mg of one or more pregenomic RNA encapsidation inhibitors according to the present invention and one or more excipients; and from about 0.1 mg to about 10 mg of one or more pregenomic RNA encapsidation inhibitors according to the present invention; and one or more excipients.

### Procedures

The following procedures can be utilized in evaluating and selecting compounds as the pregenomic RNA encapsidation inhibitors of HBV.

The HBV replication inhibitors of the present invention are capable of treating and preventing diseases associated with HBV infection. The results presented in Table 2 demonstrated that compounds of the present invention inhibit HBV replication in an immortalized murine hepatocyte (AML12)-derived stable cell line (AML12HBV10) that supports robust HBV replication in a tetracycline inducible manner without measurable cytotoxicity up to 50 μM by using the standard MTT assay (Promega).

The antiviral efficacy of the compounds of the disclosure, as presented in Table 2, were determined in AML12HBV10 cells. AML12HBV10 is an immortalized murine hepatocyte (AML12)-derived stable cell line that supports robust HBV replication in a tetracycline inducible manner (Xu et al.). The cells were seeded into 96 well plates at a density of 2×10⁴ cells per well and cultured in DMEM/F12 media with 10% fetal bovine serum in the absence of tetracycline to allow pgRNA transcription and HBV DNA replication. One day after seeding, cells were left untreated or treated with a serial dilution of testing compounds, ranging from 50 μM to 0.39 μM, for 48 hours. Cells were then lysed by adding into each well of 100 μl lysis buffer containing 10 mM Tris-HCl (pH 7.6), 1 mM EDTA, 100 mM NaCl and 1% NP-40 and incubated at 37° C. for 30 minutes. Half amount (50 μλ) of cell lysate from each well was combined with equal volume of denaturing solution containing 0.5N NaOH and 1.5M NaCl. After 5 minute incubation, 100 μl of neutralization solution (1M Tris-HCl, pH 7.4, 1.5M NaCl) was added into each well. The denatured cell lysates (totally 200 μl) were applied onto Nylon membrane using 96-well dot-blot manifold (Biorad). HBV DNA in the cell lysates were determined by dot-blot hybridization with alpha-³²P-UTP-labelled riboprobe specific for HBV minus strand DNA. The antiviral efficacy of a compound of the disclosure was expressed as the concentration that reduces the amount of HBV DNA by 50% (EC₅₀).

Determination of cytotoxicity of compounds of the disclosure in AML12HBV10 cells: To determine the cytotoxicity of the compounds, AML12HBV10 cells were seeded into 96-well plates at a density of 2×10⁴ cells per well and cultured in DMEM/F12 media with 10% fetal bovine serum in the absence of tetracycline to allow pgRNA transcription and HBV DNA replication. One day after seeding, cells were left untreated or treated with a serial dilution of testing compounds, ranging from 50 μM to 0.39 μM, for 48 hours. The cell viability was measured by a MTT assay, following procedure provided by the manufacturer (Promega). The cytotoxicity of a compound was expressed as the concentration of compound that reduces the viability of the cells by 50% (CC₅₀).

TABLE 2
Antiviral activity (EC₅₀) of exemplary compounds of the disclosure

| | | EC₅₀ |
|---|---|---|
| Entry | Structure | (□M) |
| 1 | 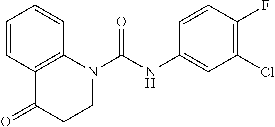 [SMILES: O=C1CCN(C(=O)Nc2ccc(F)c(Cl)c2)c2ccccc21] | 1.32 |
| 2 | 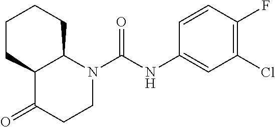 [SMILES: O=C1CCN(C(=O)Nc2ccc(F)c(Cl)c2)[C@@H]2CCCC[C@H]12] | 0.42 |
| 3 | 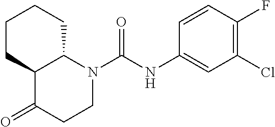 [SMILES: O=C1CCN(C(=O)Nc2ccc(F)c(Cl)c2)[C@H]2CCCC[C@H]12] | 0.42 |
| 4 | 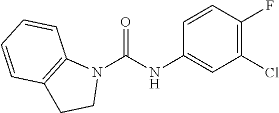 [SMILES: O=C(Nc1ccc(F)c(Cl)c1)N1CCc2ccccc21] | 0.36 |
| 5 | 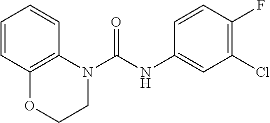 [SMILES: O=C(Nc1ccc(F)c(Cl)c1)N1CCOc2ccccc21] | 0.52 |
| 6 | 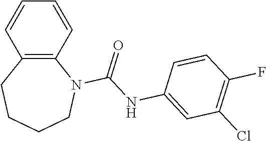 [SMILES: O=C(Nc1ccc(F)c(Cl)c1)N1CCCCc2ccccc21] | 12.3 |
| 7 | 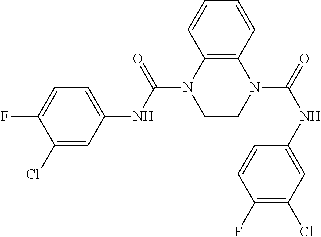 [SMILES: O=C(Nc1ccc(F)c(Cl)c1)N1CCN(C(=O)Nc2ccc(F)c(Cl)c2)c2ccccc21] | 1.01 |
| 8 | 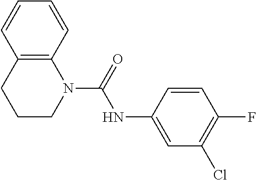 [SMILES: O=C(Nc1ccc(F)c(Cl)c1)N1CCCc2ccccc21] | 1.55 |
| 9 | 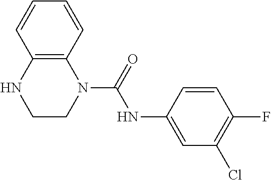 [SMILES: O=C(Nc1ccc(F)c(Cl)c1)N1CCNc2ccccc21] | 0.50 |
| 10 | 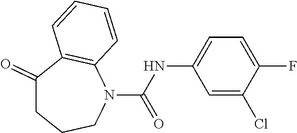 [SMILES: O=C1CCCN(C(=O)Nc2ccc(F)c(Cl)c2)c2ccccc21] | 10 |
| 11 | 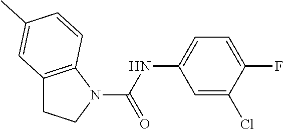 [SMILES: Cc1ccc2c(c1)CCN2C(=O)Nc1ccc(F)c(Cl)c1] | 5.26 |
| 12 | 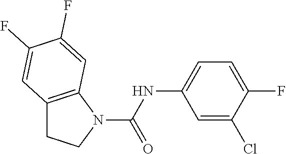 [SMILES: O=C(Nc1ccc(F)c(Cl)c1)N1CCc2cc(F)c(F)cc21] | 2.07 |
| 13 | 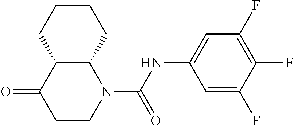 [SMILES: O=C1CCN(C(=O)Nc2cc(F)c(F)c(F)c2)[C@H]2CCCC[C@@H]12] | 2.41 |
| 14 | 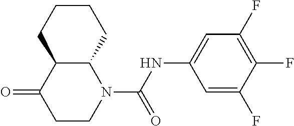 [SMILES: O=C1CCN(C(=O)Nc2cc(F)c(F)c(F)c2)[C@H]2CCCC[C@H]12] | 3.46 |
| 15 | 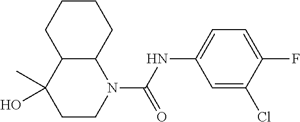 [SMILES: CC1(O)CCN(C(=O)Nc2ccc(F)c(Cl)c2)C2CCCCC21] | 6.40 |
| 16 | 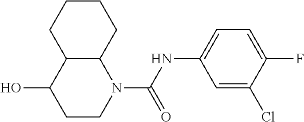 [SMILES: O=C(Nc1ccc(F)c(Cl)c1)N1CCC(O)C2CCCCC21] | 10 |
| 17 | 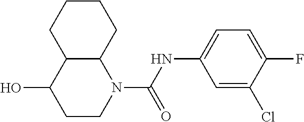 [SMILES: O=C(Nc1ccc(F)c(Cl)c1)N1CCC(O)C2CCCCC21] | 6.14 |
| 18 | 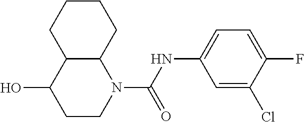 [SMILES: O=C(Nc1ccc(F)c(Cl)c1)N1CCC(O)C2CCCCC21] | 2.7 |
| 19 | 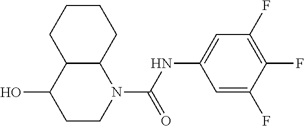 [SMILES: O=C(Nc1cc(F)c(F)c(F)c1)N1CCC(O)C2CCCCC21] | 10 |
| 20 | 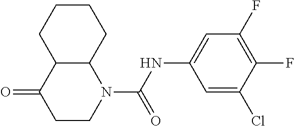 [SMILES: O=C1CCN(C(=O)Nc2cc(F)c(F)c(Cl)c2)C2CCCCC12] | 2.4 |
| 21 | 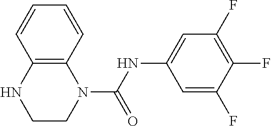 [SMILES: O=C(Nc1cc(F)c(F)c(F)c1)N1CCNc2ccccc21] | 0.63 |
| 22 | 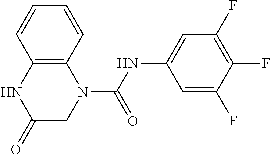 [SMILES: O=C1CN(C(=O)Nc2cc(F)c(F)c(F)c2)c2ccccc2N1] | 10 |
| 23 | 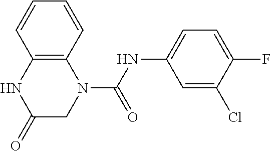 [SMILES: O=C1CN(C(=O)Nc2ccc(F)c(Cl)c2)c2ccccc2N1] | 10 |
| 24 | 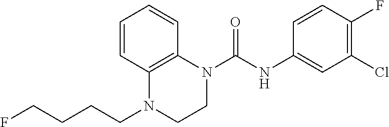 [SMILES: O=C(Nc1ccc(F)c(Cl)c1)N1CCN(CCCCF)c2ccccc21] | 10 |
| 25 | 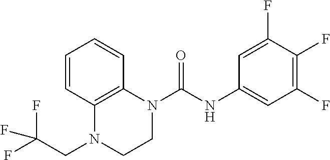 [SMILES: O=C(Nc1cc(F)c(F)c(F)c1)N1CCN(CC(F)(F)F)c2ccccc21] | 10 |
| 26 | 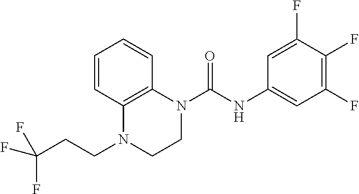 [SMILES: O=C(Nc1cc(F)c(F)c(F)c1)N1CCN(CCC(F)(F)F)c2ccccc21] | 10 |
| 27 | 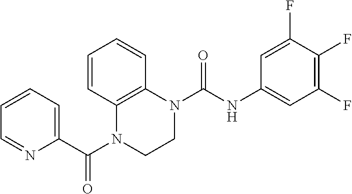 [SMILES: O=C(Nc1cc(F)c(F)c(F)c1)N1CCN(C(=O)c2ccccn2)c2ccccc21] | 10 |
| 28 | 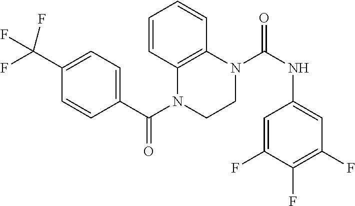 [SMILES: O=C(Nc1cc(F)c(F)c(F)c1)N1CCN(C(=O)c2ccc(C(F)(F)F)cc2)c2ccccc21] | 10 |
| 29 | 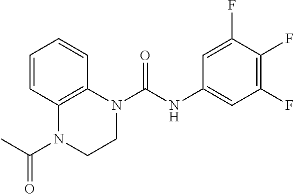 [SMILES: CC(=O)N1CCN(C(=O)Nc2cc(F)c(F)c(F)c2)c2ccccc21] | 10 |
| 30 | 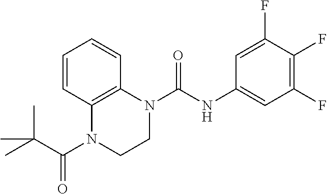 [SMILES: CC(C)(C)C(=O)N1CCN(C(=O)Nc2cc(F)c(F)c(F)c2)c2ccccc21] | 10 |
| 31 | 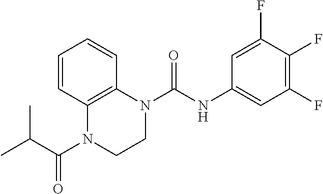 [SMILES: CC(C)C(=O)N1CCN(C(=O)Nc2cc(F)c(F)c(F)c2)c2ccccc21] | 4.709 |
| 32 | 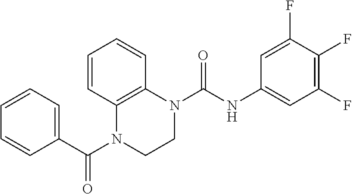 [SMILES: O=C(Nc1cc(F)c(F)c(F)c1)N1CCN(C(=O)c2ccccc2)c2ccccc21] | 10 |
| 33 | 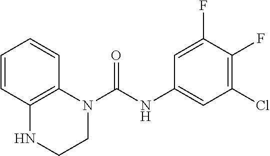 [SMILES: O=C(Nc1cc(F)c(F)c(Cl)c1)N1CCNc2ccccc21] | 0.93 |
| 34 | 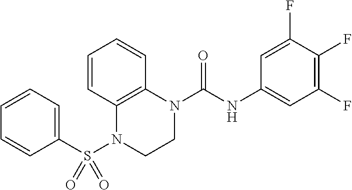 [SMILES: O=C(Nc1cc(F)c(F)c(F)c1)N1CCN(S(=O)(=O)c2ccccc2)c2ccccc21] | 10 |
| 35 | 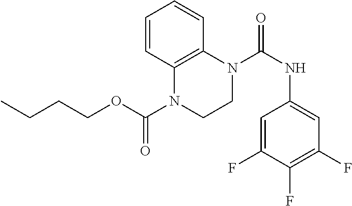 [SMILES: CCCCOC(=O)N1CCN(C(=O)Nc2cc(F)c(F)c(F)c2)c2ccccc21] | 10 |
| 36 | 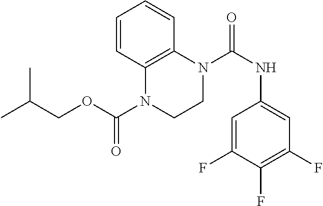 [SMILES: CC(C)COC(=O)N1CCN(C(=O)Nc2cc(F)c(F)c(F)c2)c2ccccc21] | 10 |
| 37 | 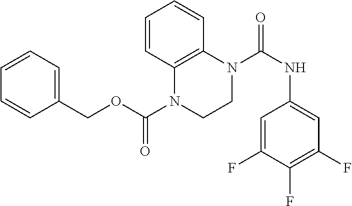 [SMILES: O=C(Nc1cc(F)c(F)c(F)c1)N1CCN(C(=O)OCc2ccccc2)c2ccccc21] | 10 |
| 38 | 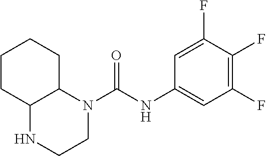 [SMILES: O=C(Nc1cc(F)c(F)c(F)c1)N1CCNC2CCCCC21] | 10 |
| 39 | 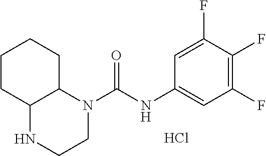 [SMILES: O=C(Nc1cc(F)c(F)c(F)c1)N1CCNC2CCCCC21 ; Cl] | 10 |
| 40 | 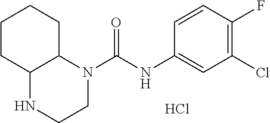 [SMILES: O=C(Nc1ccc(F)c(Cl)c1)N1CCNC2CCCCC21 ; Cl] | 10 |
| 41 | 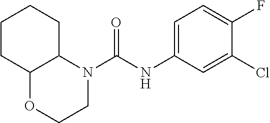 [SMILES: O=C(Nc1ccc(F)c(Cl)c1)N1CCOC2CCCCC21] | 10 |
| 42 | 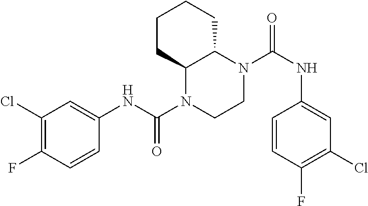 [SMILES: O=C(Nc1ccc(F)c(Cl)c1)N1CCN(C(=O)Nc2ccc(F)c(Cl)c2)[C@H]2CCCC[C@@H]21] | 10 |
| 43 | 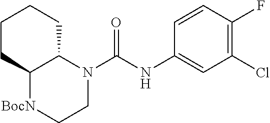 [SMILES: CC(C)(C)OC(=O)N1CCN(C(=O)Nc2ccc(F)c(Cl)c2)[C@H]2CCCC[C@@H]21] | 10 |
| 44 | 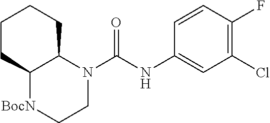 [SMILES: CC(C)(C)OC(=O)N1CCN(C(=O)Nc2ccc(F)c(Cl)c2)[C@@H]2CCCC[C@@H]21] | 10 |
| 45 | 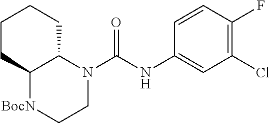 [SMILES: CC(C)(C)OC(=O)N1CCN(C(=O)Nc2ccc(F)c(Cl)c2)[C@H]2CCCC[C@@H]21] | 10 |
| 46 | 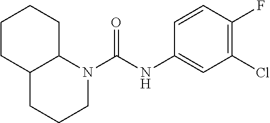 [SMILES: O=C(Nc1ccc(F)c(Cl)c1)N1CCCC2CCCCC21] | 10 |
| 47 | 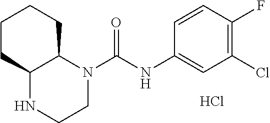 [SMILES: O=C(Nc1ccc(F)c(Cl)c1)N1CCN[C@H]2CCCC[C@H]21 ; Cl] | 10 |
| 48 | 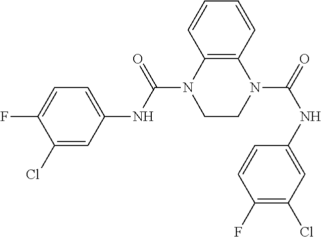 [SMILES: O=C(Nc1ccc(F)c(Cl)c1)N1CCN(C(=O)Nc2ccc(F)c(Cl)c2)c2ccccc21] | 0.75 |
| 49 | 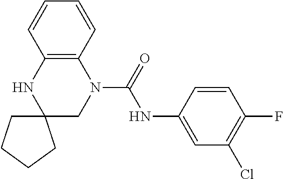 [SMILES: O=C(Nc1ccc(F)c(Cl)c1)N1CC2(CCCC2)Nc2ccccc21] | 10 |
| 50 | 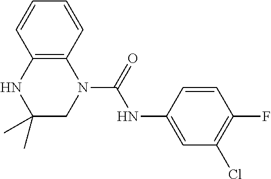 [SMILES: CC1(C)CN(C(=O)Nc2ccc(F)c(Cl)c2)c2ccccc2N1] | 10 |
| 51 | 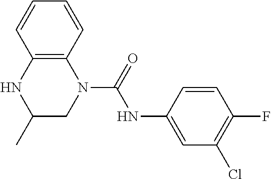 [SMILES: CC1CN(C(=O)Nc2ccc(F)c(Cl)c2)c2ccccc2N1] | 10 |
| 52 | 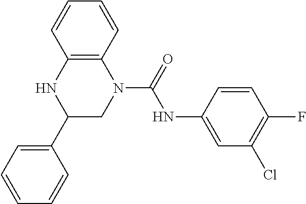 [SMILES: O=C(Nc1ccc(F)c(Cl)c1)N1CC(c2ccccc2)Nc2ccccc21] | 10 |
| 53 | 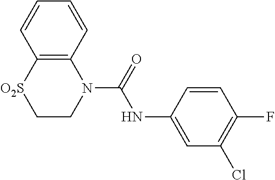 [SMILES: O=C(Nc1ccc(F)c(Cl)c1)N1CCS(=O)(=O)c2ccccc21] | 10 |
| 54 | 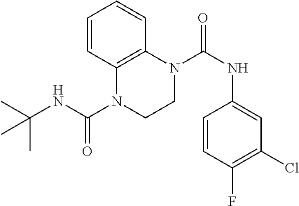 [SMILES: CC(C)(C)NC(=O)N1CCN(C(=O)Nc2ccc(F)c(Cl)c2)c2ccccc21] | 10 |
| 55 | 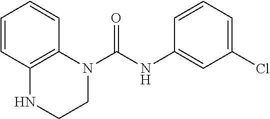 [SMILES: O=C(Nc1cccc(Cl)c1)N1CCNc2ccccc21] | 2.1 |
| 56 | 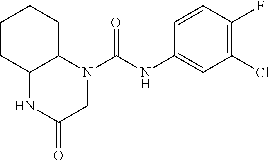 [SMILES: O=C1CN(C(=O)Nc2ccc(F)c(Cl)c2)C2CCCCC2N1] | 10 |
| 57 | 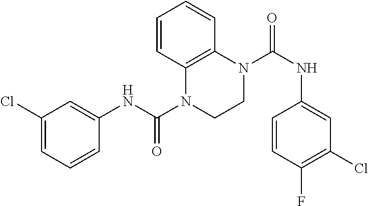 [SMILES: O=C(Nc1cccc(Cl)c1)N1CCN(C(=O)Nc2ccc(F)c(Cl)c2)c2ccccc21] | 0.65 |
| 58 | 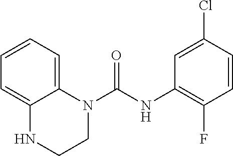 [SMILES: O=C(Nc1cc(Cl)ccc1F)N1CCNc2ccccc21] | 0.77 |
| 59 | 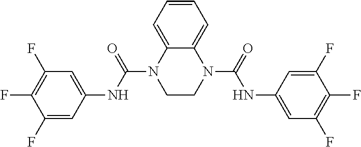 [SMILES: O=C(Nc1cc(F)c(F)c(F)c1)N1CCN(C(=O)Nc2cc(F)c(F)c(F)c2)c2ccccc21] | 1.12 |
| 60 | 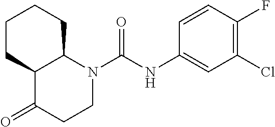 [SMILES: O=C1CCN(C(=O)Nc2ccc(F)c(Cl)c2)[C@@H]2CCCC[C@H]12] | 2.07 |
| 61 | 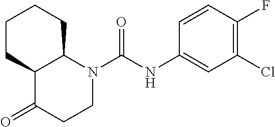 [SMILES: O=C1CCN(C(=O)Nc2ccc(F)c(Cl)c2)[C@@H]2CCCC[C@H]12] | 0.86 |
| 62 | 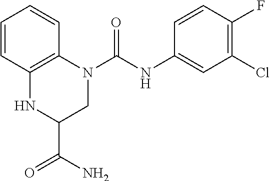 [SMILES: NC(=O)C1CN(C(=O)Nc2ccc(F)c(Cl)c2)c2ccccc2N1] | 10 |
| 63 | 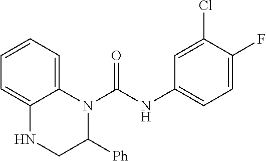 [SMILES: O=C(Nc1ccc(F)c(Cl)c1)N1c2ccccc2NCC1c1ccccc1] | 0.78 |
| 64 | 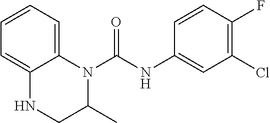 [SMILES: CC1CNc2ccccc2N1C(=O)Nc1ccc(F)c(Cl)c1] | 4.38 |
| 65 | 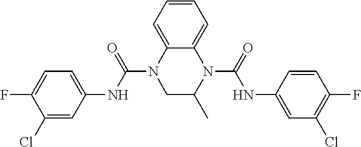 [SMILES: CC1CN(C(=O)Nc2ccc(F)c(Cl)c2)c2ccccc2N1C(=O)Nc1ccc(F)c(Cl)c1] | 1.3 |
| 66 | 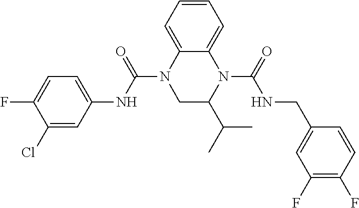 [SMILES: CC(C)C1CN(C(=O)Nc2ccc(F)c(Cl)c2)c2ccccc2N1C(=O)NCc1ccc(F)c(F)c1] | 1.66 |
| 67 | 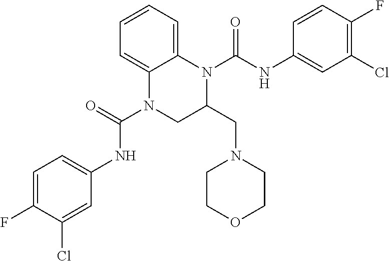 [SMILES: O=C(Nc1ccc(F)c(Cl)c1)N1CC(CN2CCOCC2)N(C(=O)Nc2ccc(F)c(Cl)c2)c2ccccc21] | 2.03 |
| 68 | 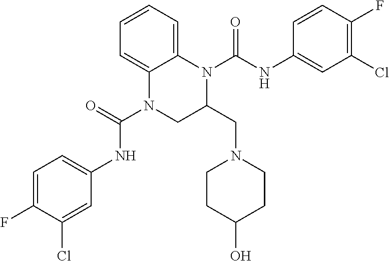 [SMILES: O=C(Nc1ccc(F)c(Cl)c1)N1CC(CN2CCC(O)CC2)N(C(=O)Nc2ccc(F)c(Cl)c2)c2ccccc21] | 10 |
| 69 | 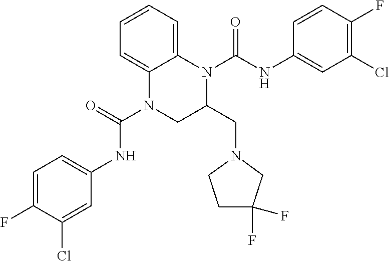 [SMILES: O=C(Nc1ccc(F)c(Cl)c1)N1CC(CN2CCC(F)(F)C2)N(C(=O)Nc2ccc(F)c(Cl)c2)c2ccccc21] | 0.65 |
| 70 | 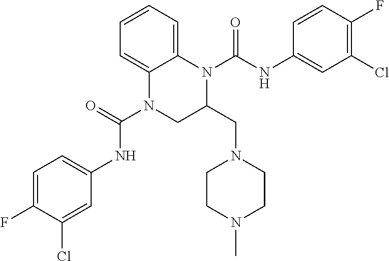 [SMILES: CN1CCN(CC2CN(C(=O)Nc3ccc(F)c(Cl)c3)c3ccccc3N2C(=O)Nc2ccc(F)c(Cl)c2)CC1] | 10 |
| 71 | 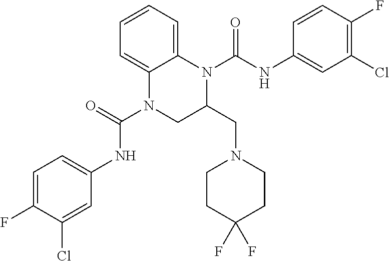 [SMILES: O=C(Nc1ccc(F)c(Cl)c1)N1CC(CN2CCC(F)(F)CC2)N(C(=O)Nc2ccc(F)c(Cl)c2)c2ccccc21] | 0.72 |
| 72 | 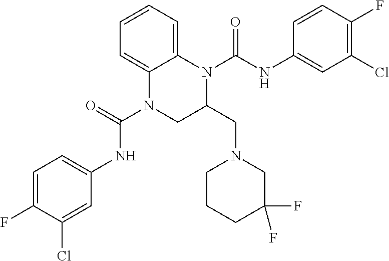 [SMILES: O=C(Nc1ccc(F)c(Cl)c1)N1CC(CN2CCCC(F)(F)C2)N(C(=O)Nc2ccc(F)c(Cl)c2)c2ccccc21] | 1.23 |
| 73 | 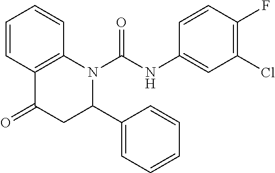 [SMILES: O=C1CC(c2ccccc2)N(C(=O)Nc2ccc(F)c(Cl)c2)c2ccccc21] | 2.89 |
| 74 | 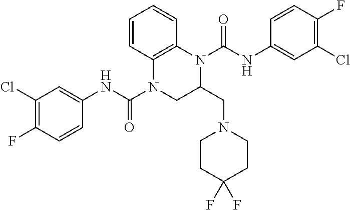 [SMILES: O=C(Nc1ccc(F)c(Cl)c1)N1CC(CN2CCC(F)(F)CC2)N(C(=O)Nc2ccc(F)c(Cl)c2)c2ccccc21] | 1.61 |
| 75 | 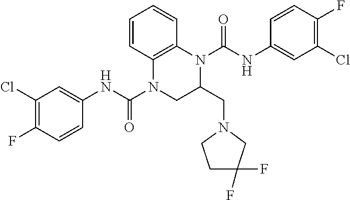 [SMILES: O=C(Nc1ccc(F)c(Cl)c1)N1CC(CN2CCC(F)(F)C2)N(C(=O)Nc2ccc(F)c(Cl)c2)c2ccccc21] | 1.14 |
| 76 | 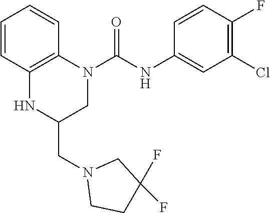 [SMILES: O=C(Nc1ccc(F)c(Cl)c1)N1CC(CN2CCC(F)(F)C2)Nc2ccccc21] | 5.09 |
| 77 | 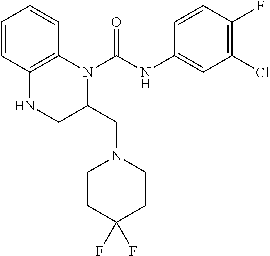 [SMILES: O=C(Nc1ccc(F)c(Cl)c1)N1c2ccccc2NCC1CN1CCC(F)(F)CC1] | 0.28 |
| 78 | 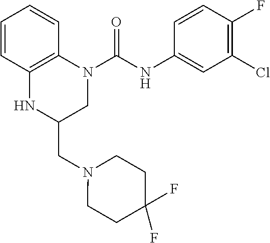 [SMILES: O=C(Nc1ccc(F)c(Cl)c1)N1CC(CN2CCC(F)(F)CC2)Nc2ccccc21] | 4.32 |
| 79 | 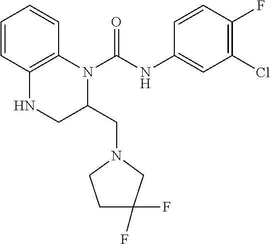 [SMILES: O=C(Nc1ccc(F)c(Cl)c1)N1c2ccccc2NCC1CN1CCC(F)(F)C1] | 1.7 |
| 80 | 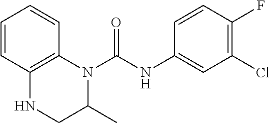 [SMILES: CC1CNc2ccccc2N1C(=O)Nc1ccc(F)c(Cl)c1] | 3.02 |
| 81 | 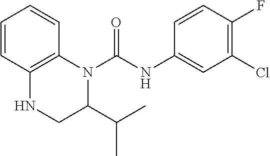 [SMILES: CC(C)C1CNc2ccccc2N1C(=O)Nc1ccc(F)c(Cl)c1] | 3.5 |
| 82 | 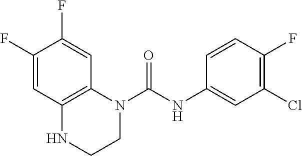 [SMILES: O=C(Nc1ccc(F)c(Cl)c1)N1CCNc2cc(F)c(F)cc21] | 0.61 |
| 83 | 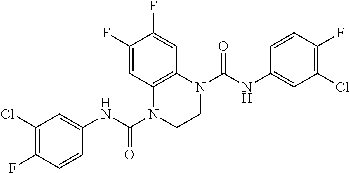 [SMILES: O=C(Nc1ccc(F)c(Cl)c1)N1CCN(C(=O)Nc2ccc(F)c(Cl)c2)c2cc(F)c(F)cc21] | 1.18 |
| 84 | 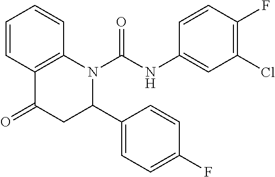 [SMILES: O=C1CC(c2ccc(F)cc2)N(C(=O)Nc2ccc(F)c(Cl)c2)c2ccccc21] | 2.37 |
| 85 | 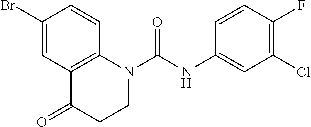 [SMILES: O=C1CCN(C(=O)Nc2ccc(F)c(Cl)c2)c2ccc(Br)cc21] | 10 |
| 86 | 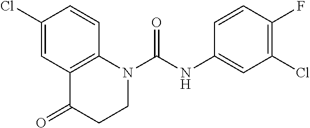 [SMILES: O=C1CCN(C(=O)Nc2ccc(F)c(Cl)c2)c2ccc(Cl)cc21] | 4.83 |
| 87 | 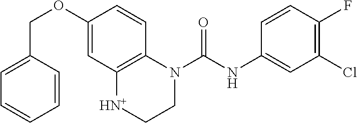 [SMILES: O=C(Nc1ccc(F)c(Cl)c1)N1CC[NH2+]c2cc(OCc3ccccc3)ccc21] | 2.82 |
| 88 | 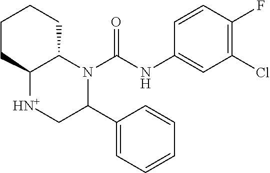 [SMILES: O=C(Nc1ccc(F)c(Cl)c1)N1C(c2ccccc2)C[NH2+][C@H]2CCCC[C@@H]21] | 10 |
| 89 | 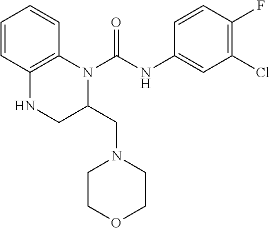 [SMILES: O=C(Nc1ccc(F)c(Cl)c1)N1c2ccccc2NCC1CN1CCOCC1] | 7.3 |
| 90 | 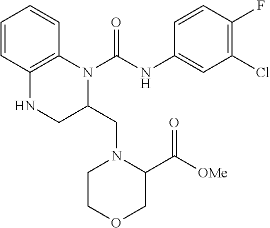 [SMILES: COC(=O)C1COCCN1CC1CNc2ccccc2N1C(=O)Nc1ccc(F)c(Cl)c1] | 2.2 |
| 91 | 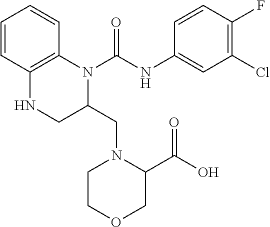 [SMILES: O=C(O)C1COCCN1CC1CNc2ccccc2N1C(=O)Nc1ccc(F)c(Cl)c1] | 10 |
| 92 | 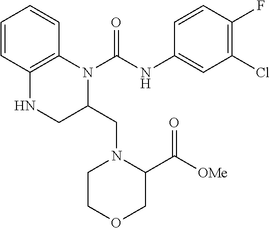 [SMILES: COC(=O)C1COCCN1CC1CNc2ccccc2N1C(=O)Nc1ccc(F)c(Cl)c1] | 2.29 |
| 93 | 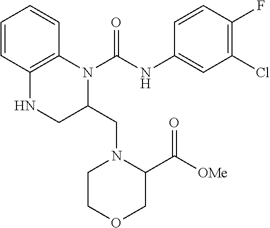 [SMILES: COC(=O)C1COCCN1CC1CNc2ccccc2N1C(=O)Nc1ccc(F)c(Cl)c1] | 3.35 |
| 94 | 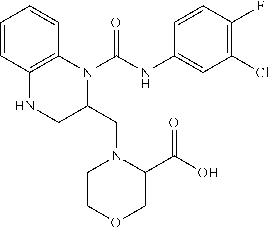 [SMILES: O=C(O)C1COCCN1CC1CNc2ccccc2N1C(=O)Nc1ccc(F)c(Cl)c1] | 10 |
| 95 | 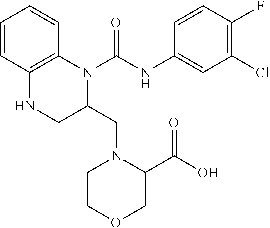 [SMILES: O=C(O)C1COCCN1CC1CNc2ccccc2N1C(=O)Nc1ccc(F)c(Cl)c1] | 10 |
| 96 | 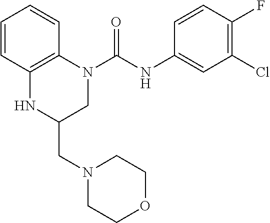 [SMILES: O=C(Nc1ccc(F)c(Cl)c1)N1CC(CN2CCOCC2)Nc2ccccc21] | 10 |
| 97 | 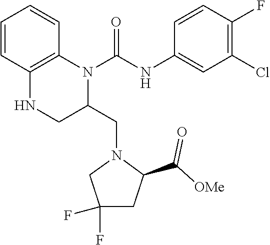 [SMILES: COC(=O)[C@H]1CC(F)(F)CN1CC1CNc2ccccc2N1C(=O)Nc1ccc(F)c(Cl)c1] | 10 |
| 98 | 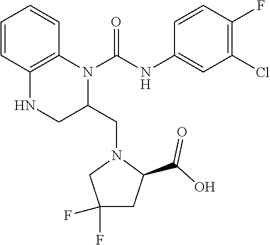 [SMILES: O=C(O)[C@H]1CC(F)(F)CN1CC1CNc2ccccc2N1C(=O)Nc1ccc(F)c(Cl)c1] | 10 |
| 99 | 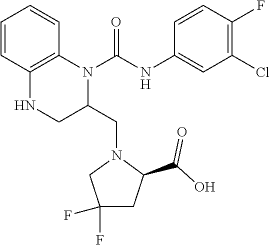 [SMILES: O=C(O)[C@H]1CC(F)(F)CN1CC1CNc2ccccc2N1C(=O)Nc1ccc(F)c(Cl)c1] | 10 |
| 100 | 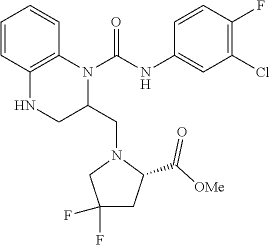 [SMILES: COC(=O)[C@@H]1CC(F)(F)CN1CC1CNc2ccccc2N1C(=O)Nc1ccc(F)c(Cl)c1] | 1.11 |
| 101 | 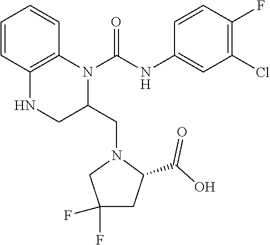 [SMILES: O=C(O)[C@@H]1CC(F)(F)CN1CC1CNc2ccccc2N1C(=O)Nc1ccc(F)c(Cl)c1] | 2.2 |
| 102 | 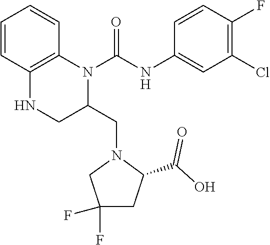 [SMILES: O=C(O)[C@@H]1CC(F)(F)CN1CC1CNc2ccccc2N1C(=O)Nc1ccc(F)c(Cl)c1] | 10 |
| 103 | 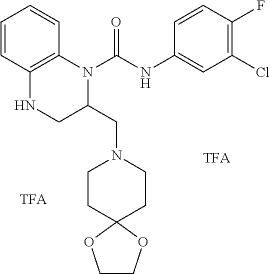 [SMILES: O=C(Nc1ccc(F)c(Cl)c1)N1c2ccccc2NCC1CN1CCC2(CC1)OCCO2] | 10 |
| 104 | 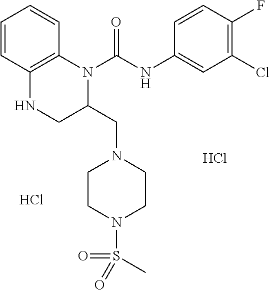 [SMILES: CS(=O)(=O)N1CCN(CC2CNc3ccccc3N2C(=O)Nc2ccc(F)c(Cl)c2)CC1 ; Cl ; Cl] | 10 |
| 105 | 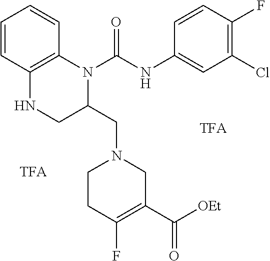 [SMILES: CCOC(=O)C1=C(F)CCN(CC2CNc3ccccc3N2C(=O)Nc2ccc(F)c(Cl)c2)C1] | 10 |
| 106 | 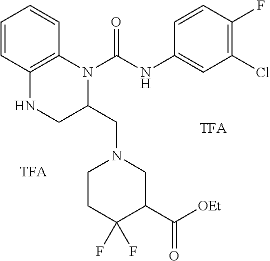 [SMILES: CCOC(=O)C1CN(CC2CNc3ccccc3N2C(=O)Nc2ccc(F)c(Cl)c2)CCC1(F)F] | 7.68 |
| 107 | 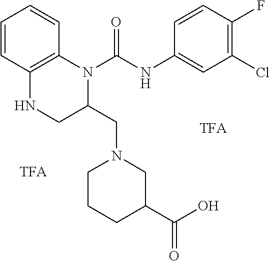 [SMILES: O=C(O)C1CCCN(CC2CNc3ccccc3N2C(=O)Nc2ccc(F)c(Cl)c2)C1] | 10 |
| 108 | 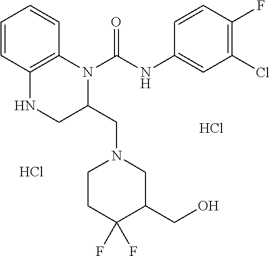 [SMILES: O=C(Nc1ccc(F)c(Cl)c1)N1c2ccccc2NCC1CN1CCC(F)(F)C(CO)C1 ; Cl ; Cl] | 2.17 |
| 109 | 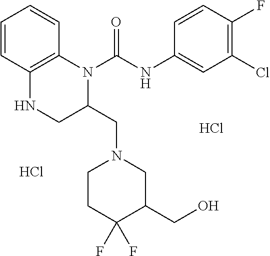 [SMILES: O=C(Nc1ccc(F)c(Cl)c1)N1c2ccccc2NCC1CN1CCC(F)(F)C(CO)C1 ; Cl ; Cl] | 1.99 |
| 110 | 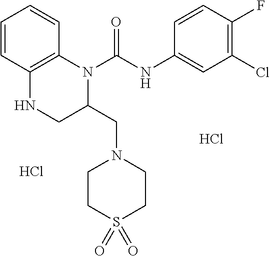 [SMILES: O=C(Nc1ccc(F)c(Cl)c1)N1c2ccccc2NCC1CN1CCS(=O)(=O)CC1 ; Cl ; Cl] | 2.0 |
| 111 | 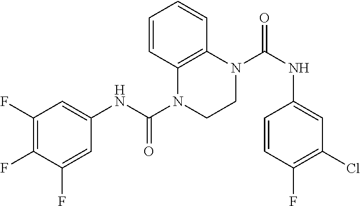 [SMILES: O=C(Nc1cc(F)c(F)c(F)c1)N1CCN(C(=O)Nc2ccc(F)c(Cl)c2)c2ccccc21] | 0.50 |
| 112 | 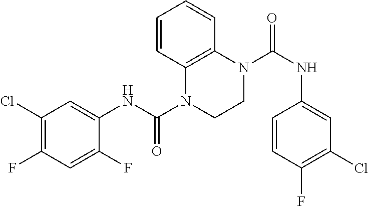 [SMILES: O=C(Nc1ccc(F)c(Cl)c1)N1CCN(C(=O)Nc2cc(Cl)c(F)cc2F)c2ccccc21] | 0.29 |
| 113 | 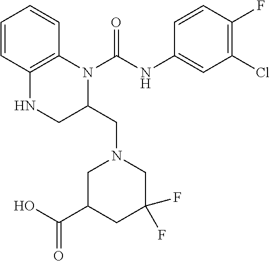 [SMILES: O=C(O)C1CN(CC2CNc3ccccc3N2C(=O)Nc2ccc(F)c(Cl)c2)CC(F)(F)C1] | 0.45 |
| 114 | 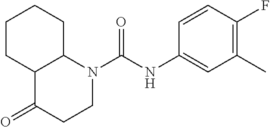 [SMILES: Cc1cc(NC(=O)N2CCC(=O)C3CCCCC32)ccc1F] | 1.01 |
| 115 | 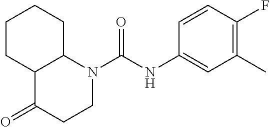 [SMILES: Cc1cc(NC(=O)N2CCC(=O)C3CCCCC32)ccc1F] | 0.39 |
| 116 | 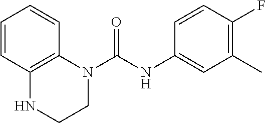 [SMILES: Cc1cc(NC(=O)N2CCNc3ccccc32)ccc1F] | 0.24 |
| 117 | 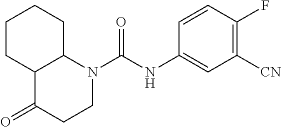 [SMILES: N#Cc1cc(NC(=O)N2CCC(=O)C3CCCCC32)ccc1F] | 1.97 |
| 118 | 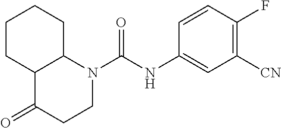 [SMILES: N#Cc1cc(NC(=O)N2CCC(=O)C3CCCCC32)ccc1F] | 0.85 |
| 119 | 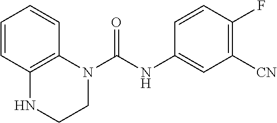 [SMILES: N#Cc1cc(NC(=O)N2CCNc3ccccc32)ccc1F] | 0.52 |
| 120 | 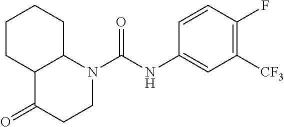 [SMILES: O=C1CCN(C(=O)Nc2ccc(F)c(C(F)(F)F)c2)C2CCCCC12] | 1.09 |
| 121 | 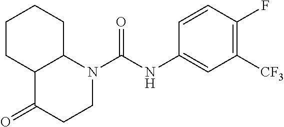 [SMILES: O=C1CCN(C(=O)Nc2ccc(F)c(C(F)(F)F)c2)C2CCCCC12] | 0.98 |
| 122 | 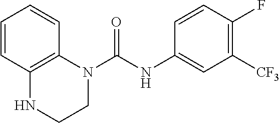 [SMILES: O=C(Nc1ccc(F)c(C(F)(F)F)c1)N1CCNc2ccccc21] | 0.32 |
| 123 | 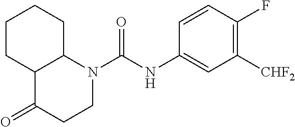 [SMILES: O=C1CCN(C(=O)Nc2ccc(F)c(C(F)F)c2)C2CCCCC12] | 0.56 |
| 124 | 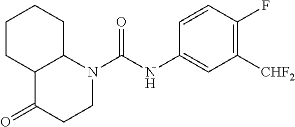 [SMILES: O=C1CCN(C(=O)Nc2ccc(F)c(C(F)F)c2)C2CCCCC12] | 0.27 |
| 125 | 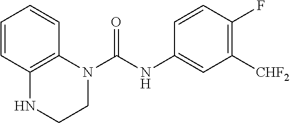 [SMILES: O=C(Nc1ccc(F)c(C(F)F)c1)N1CCNc2ccccc21] | 0.19 |
| 126 | 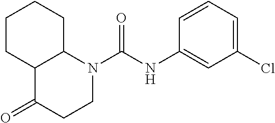 [SMILES: O=C1CCN(C(=O)Nc2cccc(Cl)c2)C2CCCCC12] | 0.20 |
| 127 | 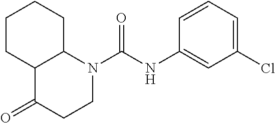 [SMILES: O=C1CCN(C(=O)Nc2cccc(Cl)c2)C2CCCCC12] | 0.22 |
| 128 | 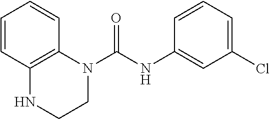 [SMILES: O=C(Nc1cccc(Cl)c1)N1CCNc2ccccc21] | 0.20 |

What is claimed:

1) A compound having the formula (I) 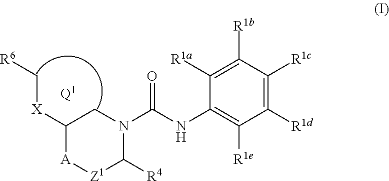
Including enantiomers, diasteromers, hydrates, solvates, pharmaceutically acceptable salts, isotopic analogs, prodrugs and complexes thereof, wherein:
A is selected from a group consisting of CH₂, carbonyl (C═O), oxygen, 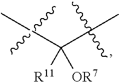
and NR^{7a};
Z¹ is selected from the group consisting of a bond, carbonyl (C═O), 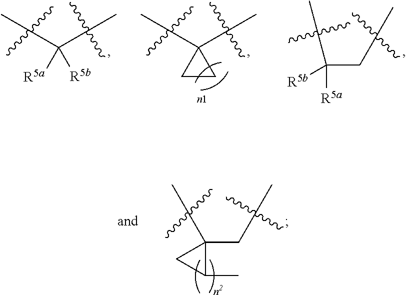
n¹ is 1, 2, 3, 4, or 5;
n² is 1, 2, 3, 4, or 5;
When A is carbonyl (C═O), Z¹ is not carbonyl (C═O);
The ring designated Q¹ is selected from the group consisting of an aromatic ring containing 5 ring atoms, an aromatic ring containing 6 ring atoms, a saturated ring containing 5 ring atoms, a saturated ring containing 6 ring atoms, and a saturated ring containing 7 ring atoms;
When the ring designated Q¹ is an aromatic ring containing 6 ring atoms, X is selected from the group consisting CR⁸ and nitrogen;
When the ring designated Q¹ is an aromatic ring containing 5 ring atoms, X is nitrogen;
When the ring designated Q¹ is a saturated ring containing 5 ring atoms, X is selected from a group consisting of CH₂, CHR⁸ and NR^{7b};
When the ring designated Q¹ is a saturated ring containing 6 ring atoms, X is selected from a group consisting of CH₂, CHR⁸ and NR^{7b};
When the ring designated Q¹ is a saturated ring containing 7 ring atoms, X is selected from a group consisting of CH₂, CHR⁸ and NR^{7b};
R^{1a} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1b} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1c} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1d} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1e} is selected from the group consisting of hydrogen, fluorine, chlorine, bromine, —CHF₂, —CH₂F, —CF₃, —CN, OR⁹, C₁₋₆ alkyl and C₃₋₅ cycloalkyl;
R^{1b} and R^{1c} are optionally taken together with the atom to which they are bound to form an optionally substituted ring having 5-7 ring atoms optionally containing up to 2 groups selected from oxygen and NH;
R^{1c} and R^{1d} are optionally taken together with the atom to which they are bound to form an optionally substituted ring having 5-7 ring atoms optionally containing up to 2 groups selected from oxygen and NH;
R⁴ is selected from the group consisting of hydrogen, C₁₋₆ linear alkyl, C₃₋₇ branched alkyl, 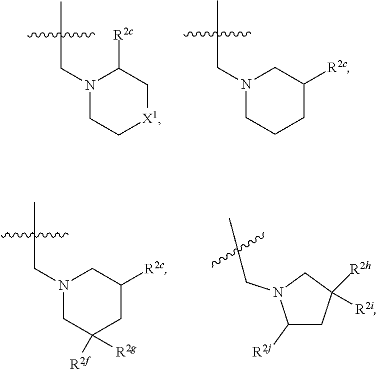
optionally substituted C₃₋₈ cycloalkyl, optionally substituted aryl, and optionally substituted heteroaryl;
X¹ is selected from the group consisting of oxygen, CR^{2a}R^{2b}, C═O, SO₂, and NSO₂R^{2d}.
R^{2a} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2b} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2c} is selected from the group consisting of hydrogen, CO₂R^{2e}, optionally substituted C₁₋₆ linear alkyl, and optionally substitute C₃₋₇ branched alkyl;
R^{2d} is selected from the group consisting of C₁₋₆ linear alkyl and C₃₋₇ branched alkyl;
R^{2e} is selected from the group consisting of hydrogen, C₁₋₆ linear alkyl, and C₃₋₇ branched alkyl;
R^{2f} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2g} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2h} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R²ⁱ is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{2j} is selected from the group consisting of hydrogen, CO₂R^{2e}, optionally substituted C₁₋₆ linear alkyl, and optionally substitute C₃₋₇ branched alkyl;
R^{5a} is selected from the group consisting of hydrogen, C₁₋₆ linear alkyl, C₃₋₇ branched alkyl, CONH₂, 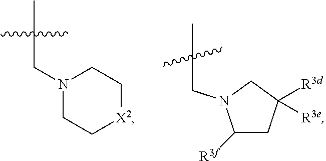
optionally substituted aryl, and optionally substituted heteroaryl;
R^{5b} is selected from a group consisting of hydrogen, C₁₋₆ linear alkyl, and C₃₋₇ branched alkyl;
X² is selected from the group consisting of oxygen, CR^{3a}R^{3b}, C═O, SO₂, NSO₂R^{3c},
R^{3a} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3b} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3c} is selected from the group consisting of C₁₋₆ linear alkyl and C₃₋₇ branched alkyl;
R^{3d} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3e} is selected from the group consisting of hydrogen, fluorine, chlorine, C₁₋₆ linear alkyl, C₃₋₇ branched alkyl, C₁₋₆ linear alkoxy, and C₃₋₇ branched alkoxy;
R^{3f} is selected from the group consisting of hydrogen, CO₂R^{3g}, optionally substituted C₁₋₆ linear alkyl, and optionally substitute C₃₋₇ branched alkyl;
R^{3g} is selected from the group consisting of hydrogen, C₁₋₆ linear alkyl, and C₃₋₇ branched alkyl;
R⁶ is selected from a group consisting of hydrogen, halogen, optionally substituted C₁₋₆ linear alkyl, optionally substituted C₃₋₇ branched alkyl, optionally substituted C₃₋₈ cycloalkyl, optionally substituted C₁₋₆ linear alkoxy, optionally substituted C₃₋₇ branched alkoxy, optionally substituted C₃₋₈ cycloalkoxy, —OBenzyl, 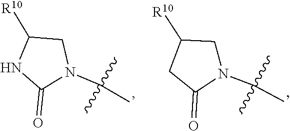
optionally substituted aryl, and optionally substituted heteroaryl;
R⁷ is selected from a group consisting of hydrogen, optionally substituted C₁₋₆ linear alkyl, optionally substituted C₃₋₇ branched alkyl, C₁₋₆ fluoroalkyl, C₃₋₇ branched fluoroalkyl optionally substituted C₁₋₆ alkenyl, CO₂R¹⁰, CONHR¹⁰, SO₂R¹⁰, and 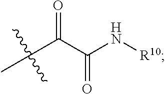
R^{7a} is selected from a group consisting of hydrogen, optionally substituted C₁₋₆ linear alkyl, optionally substituted C₃₋₇ branched alkyl, C₁₋₆ fluoroalkyl, C₃₋₇ branched fluoroalkyl, optionally substituted C₁₋₆ alkenyl, CO₂R¹⁰, CONHR¹⁰, SO₂R¹⁰, and 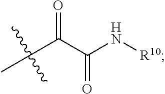
R^{7b} is selected from a group consisting of hydrogen, optionally substituted C₁₋₆ linear alkyl, optionally substituted C₃₋₇ branched alkyl, C₁₋₆ fluoroalkyl, C₃₋₇ branched fluoroalkyl, optionally substituted C₁₋₆ alkenyl, CO₂R¹⁰, CONHR¹⁰, SO₂R¹⁰, and 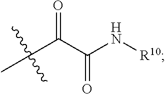
R⁸ is selected from a group consisting of hydrogen, halogen, optionally substituted C₁₋₆ linear alkyl, optionally substituted C₃₋₇ branched alkyl, optionally substituted C₃₋₈ cycloalkyl, optionally substituted aryl, optionally substituted heteroaryl, CO₂R¹⁰, CONHR¹⁰, NHCOR¹⁰, SO₂R¹⁰, and 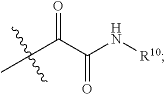
When R⁸ is 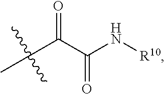
R⁷ is not 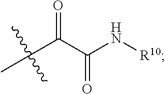
When R⁸ is 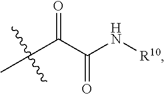
R^{7a} is not 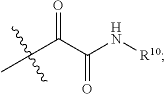
When R⁸ is 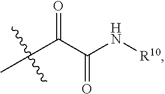
R^{7b} is not 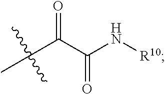
R⁹ is selected from a group consisting of hydrogen, optionally substituted C₁₋₄ alkyl, optionally substituted halo C₁₋₄ alkyl, and optionally substituted C₃₋₇ cycloalkyl;
R¹⁰ is selected from a group consisting of hydrogen, optionally substituted C₁₋₆ linear alkyl, optionally substituted C₃₋₇ branched alkyl, optionally substituted C₃₋₈ cycloalkyl, benzyl, optionally substituted aryl, and optionally substituted heteroaryl;
and
R¹¹ is selected from a group consisting of hydrogen, optionally substituted C₁₋₆ linear alkyl, optionally substituted C₃₋₇ branched alkyl, optionally substituted C₃₋₈ cycloalkyl, optionally substituted aryl, and optionally substituted heteroaryl.

2) A pharmaceutical composition comprising a compound of claim 1 and a pharmaceutically acceptable excipient.

3) A method of treating a disease that involves pregenomic RNA encapsidation, said method comprising administering to a patient in need of such treatment an effective amount of at least one compound of claim 1.

4) The method of claim 3, wherein the disease that involves pregenomic RNA encapsidation is a Hepatitis B virus infection.

5) A method of treating a Hepatitis B viral infection, said method comprising administering to a patient in need of such treatment an effective amount of at least one compound of claim 1.

6) The method of claim 5, wherein the treatment controls or ameliorates a condition associated with liver disease.

7) A method of repressing at least one process selected from the group consisting of viral replication and morphogenesis, said method comprising administering to a patient in need thereof a compound of claim 1.
